(19) United States
(12) Patent Application Publication
(10) Pub. No.: US 2024/0140949 A1
(43) Pub. Date: May 2, 2024

(54) ANTI-CANCER PHARMACEUTICAL COMPOSITION FOR TARGETING HEAT SHOCK PROTEIN 70, CONTAINING INDOLOQUINAZOLIDINE ALKALOID

(71) Applicant: SEOUL NATIONAL UNIVERSITY R & DB FOUNDATION, Seoul (KR)

(72) Inventors: Ho-Young LEE, Seoul (KR); Suckchang HONG, Seoul (KR)

(21) Appl. No.: 18/277,637

(22) PCT Filed: Feb. 17, 2022
(86) PCT No.: PCT/KR2022/002316

§ 371 Date: Aug. 17, 2023

(30) Foreign Application Priority Data
Feb. 19, 2021 (KR) 10-2021-0022639
Feb. 16, 2022 (KR) 10-2022-0020404

(51) Int. Cl. C07D 471/14; A61K 31/337; A61K 31/519; A61K 33/243; A61P 35/00

(52) U.S. Cl. CPC C07D 471/14; A61K 31/337; A61K 31/519; A61K 33/243; A61P 35/00

(57) ABSTRACT

The present invention relates to: an anti-cancer pharmaceutical composition for targeting heat shock protein 70, containing an indoloquinazolidine alkaloid; and the like. The indoloquinazolidine alkaloid of the present invention inhibits the growth of tumors and inhibits the expression of HSP70 protein and the colony formation activity of cancer cells, and inhibits the growth of tumors in mouse models with xenografts of a cancer cell line and xenografts of patient-derived cancer, and inhibits the growth of cancer cells resistant to drugs such as pemetrexed, cisplatin, and paclitaxel, and thus is expected to be widely usable in the prevention and treatment of various types of cancer.

### TECHNICAL FIELD

The present invention relates to an anti-cancer pharmaceutical composition targeting heat shock protein 70 comprising an indoloquinazolidine alkaloid, and the like.

This application claims priority based on Korean Patent Application No. 10-2021-0022639 filed on Feb. 19, 2021, and Korean Patent Application No. 10-2022-0020404 filed on Feb. 16, 2022. All contents disclosed in the specifications and drawings of these applications are incorporated herein by reference.

### BACKGROUND ART

Cancer is a major cause of death, and early diagnosis due to the development of diagnostic technology and the development of anti-cancer drugs that can effectively suppress cancer, such as immuno-oncology drugs, are being developed. However, it is essential to develop effective anti-cancer drugs because the current anti-cancer therapies are showing toxicity, side effects, decreased treatment effectiveness due to resistance, and cancer recurrence.

Lung cancer refers to malignant tumors that occur in the lungs, originating either from the lungs themselves (primary lung cancer) or from cancers that originate in other organs and metastasize to the lungs. Types of primary lung cancer are divided into non-small cell lung cancer and small cell lung cancer based on the size and shape of the cancer cells. Non-small cell lung cancer accounts for 80˜85% of lung cancers. This includes squamous cell carcinoma, adenocarcinoma, large cell carcinoma, adenosquamous carcinoma, sarcomatoid carcinoma, carcinoid tumor, salivary gland type carcinoma, and undifferentiated carcinoma. The remaining small cell lung cancer is generally highly malignant, and at the time of discovery, it is often metastasized to other organs or the opposite lung, and the mediastinum (a space between the two lungs where the heart, trachea, esophagus, aorta, etc., are located) through the lymphatic or blood vessels.

Heat shock protein 70 (Hsp70) is overexpressed in various cancer cells and is known to mediate cancer cell survival and resistance to anti-cancer drugs. Especially, recent studies have shown that when Hsp70 is inhibited, cancer initiating cells are inhibited, thereby suppressing cancer occurrence and metastasis, and Hsp70 is considered a new target for anti-cancer drug development. Several Hsp70 inhibitors are being developed, but most are still in the preclinical stage, so the development of effective Hsp70 inhibitors is needed.

Meanwhile, previous studies have reported anti-inflammatory, antiviral, hepatoprotective, anti-cancer, and cancer stem cell inhibitory properties of indoloquinazolidine alkaloids and their representative compounds, evodiamine and rutaecarpine. Also, the inventors of the present invention recently revealed the Hsp70 inhibitory effect as a new mechanism of the anti-cancer and cancer stem cell inhibitory action of evodiamine.

### DISCLOSURE

### Technical Problem

The present invention has been devised to solve the problems in the conventional art as mentioned above, and the inventors of the present invention have manufactured a novel indoloquinazolidine alkaloid having anti-cancer activity, and have confirmed the effect of preventing or treating cancer by inhibiting the proliferation of cancer cells through treatment thereof, thereby completing the present invention.

Therefore, an object of the present invention is to provide the indoloquinazolidine alkaloid represented by the following Chemical Formula 1, or a pharmaceutically acceptable salt thereof.

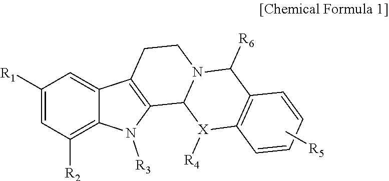

(In the Chemical Formula 1 above,
R₁ and R₂ are each independently hydrogen, substituted or unsubstituted C₁-C₆ alkyl, substituted or unsubstituted C₁-C₆ alkoxy, halogen, nitro (NO₂), amide, —NH-aryl, hydroxy, or —COOR₇,
R₃ is hydrogen, substituted or unsubstituted C₁-C₆ alkyl, —CH₂—R₈, substituted or unsubstituted C₁-C₆ acyl, substituted or unsubstituted aryl, —CO—R₈, or benzyl,
R4 is hydrogen, substituted or unsubstituted C₁-C₆ alkyl, substituted or unsubstituted C₂-C₆ alkenyl, substituted or unsubstituted C₂-C₆ alkynyl, substituted or unsubstituted C₂-C₆ alkylalkenyl, substituted or unsubstituted C₂-C₆ alkylalkynyl, —CH₂—R₈, substituted or unsubstituted aryl, propargyl, substituted or unsubstituted C₁-C₆ acyl, or benzyl,
R₅ is hydrogen, substituted or unsubstituted C₁-C₆ alkyl, halogen, nitro, or substituted or unsubstituted C₁-C₆ alkoxy,
R₆ is hydrogen, oxygen, or substituted or unsubstituted C₁-C₆ alkyl,
R₇ and R₈ are each independently hydrogen, substituted or unsubstituted C₃-C₈ cycloalkyl, substituted or unsubstituted C₁-C₆ alkyl, or substituted or unsubstituted aryl,
X is C, N, O or S.)

Another object of the present invention is to provide a pharmaceutical composition for preventing or treating cancer, which comprises the indoloquinazolidine alkaloid or a pharmaceutically acceptable salt thereof as an active ingredient.

However, the technical problem that the present invention aims to solve is not limited to the problems mentioned above, and other problems not mentioned can be clearly understood by those skilled in the art from the following description.

### Technical Solution

In order to achieve the objective of the present invention as described above, the present invention provides an indoloquinazolidine alkaloid represented by the following Chemical Formula 1, or a pharmaceutically acceptable salt thereof.

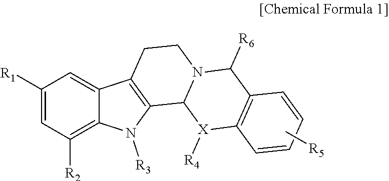

(In the Chemical Formula 1 above,
R₁ and R₂ are each independently hydrogen, substituted or unsubstituted C₁-C₆ alkyl, substituted or unsubstituted C₁-C₆ alkoxy, halogen, nitro (NO₂), amide, —NH-aryl, hydroxy, or —COOR₇,
R₃ is hydrogen, substituted or unsubstituted C₁-C₆ alkyl, —CH₂—R₈, substituted or unsubstituted C₁-C₆ acyl, substituted or unsubstituted aryl, —CO—R₈, or benzyl,
R₄ is hydrogen, substituted or unsubstituted C₁-C₆ alkyl, substituted or unsubstituted C₂-C₆ alkenyl, substituted or unsubstituted C₂-C₆ alkynyl, substituted or unsubstituted C₂-C₆ alkylalkenyl, substituted or unsubstituted C₂-C₆ alkylalkynyl, —CH₂—R₈, substituted or unsubstituted aryl, propargyl, substituted or unsubstituted C₁-C₆ acyl, or benzyl,
R₅ is hydrogen, substituted or unsubstituted C₁-C₆ alkyl, halogen, nitro, or substituted or unsubstituted C₁-C₆ alkoxy,
R₆ is hydrogen, oxygen, or substituted or unsubstituted C₁-C₆ alkyl,
R₇ and R₈ are each independently hydrogen, substituted or unsubstituted C₃-C₈ cycloalkyl, substituted or unsubstituted C₁-C₆ alkyl, or substituted or unsubstituted aryl,
X is C, N, O or S.)

Furthermore, the present invention provides a pharmaceutical composition for preventing or treating cancer, which comprises an indoloquinazolidine alkaloid or a pharmaceutically acceptable salt thereof as an active ingredient.

Moreover, the present invention provides a method for treating cancer, which comprises the step of administering to a subject in need thereof a pharmaceutical composition comprising an indoloquinazolidine alkaloid or a pharmaceutically acceptable salt thereof as an active ingredient.

Additionally, the present invention provides a use of a composition comprising an indoloquinazolidine alkaloid or a pharmaceutically acceptable salt thereof as an active ingredient, for treating cancer.

Also, the present invention provides a use of an indoloquinazolidine alkaloid or a pharmaceutically acceptable salt thereof for producing an anti-cancer drug.

In one embodiment of the present invention, the R₁ and R₂ may each independently be hydrogen, methoxy, butoxy, methyl, hydroxy, F, Cl, nitro,

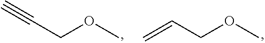

### or —COOR₇,

R₃ may be hydrogen, methyl, ethyl, propyl, butyl, hexyl, benzyl, —CO—R₈ or —CH₂—R₈,
R₄ may be hydrogen, methyl, benzyl, substituted or unsubstituted C₂-C₅ alkenyl, substituted or unsubstituted C₂-C₅ alkynyl, substituted or unsubstituted C₂-C₅ alkylalkenyl, substituted or unsubstituted C₂-C₅ alkylalkynyl, cyclohexanecarbonyl,

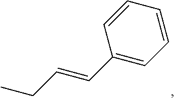

or —CH₂—R₈,
R₅ may be hydrogen, methoxy, nitro, methyl, F, Cl, Br, or I,
R₆ may be hydrogen, or oxygen,
R₇ and R₈ may each independently be hydrogen, substituted or unsubstituted C₃-C₈ cycloalkyl, substituted or unsubstituted C₁-C₆ alkyl, or substituted or unsubstituted aryl, wherein the substituted aryl may be substituted with at least one selected from the group consisting of halogen, CN, C₁-C₆ haloalkyl, C₁-C₃ alkyl, C₁-C₃ alkoxy, and nitro group,
X may be N, or O, but is not limited thereto.

In yet another embodiment of the present invention, the indoloquinazolidine alkaloid represented by Chemical Formula 1 may be one selected from the group consisting of the following compounds, but is not limited thereto.

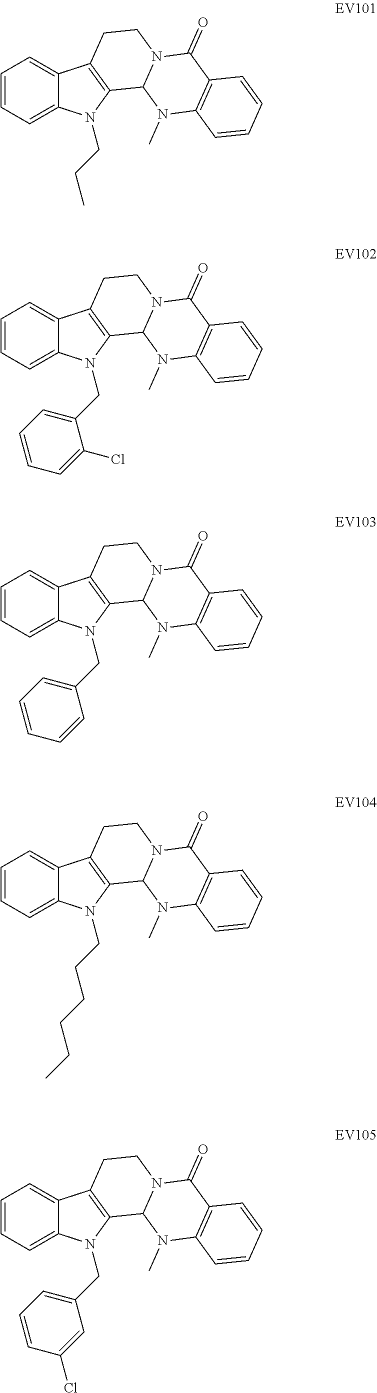 [SMILES: CCCn1c2c(c3ccccc31)CCN1C(=O)c3ccccc3N(C)C21 ; CN1c2ccccc2C(=O)N2CCc3c(n(Cc4ccccc4Cl)c4ccccc34)C21 ; CN1c2ccccc2C(=O)N2CCc3c(n(Cc4ccccc4)c4ccccc34)C21 ; CCCCCCn1c2c(c3ccccc31)CCN1C(=O)c3ccccc3N(C)C21 ; CN1c2ccccc2C(=O)N2CCc3c(n(Cc4cccc(Cl)c4)c4ccccc34)C21]

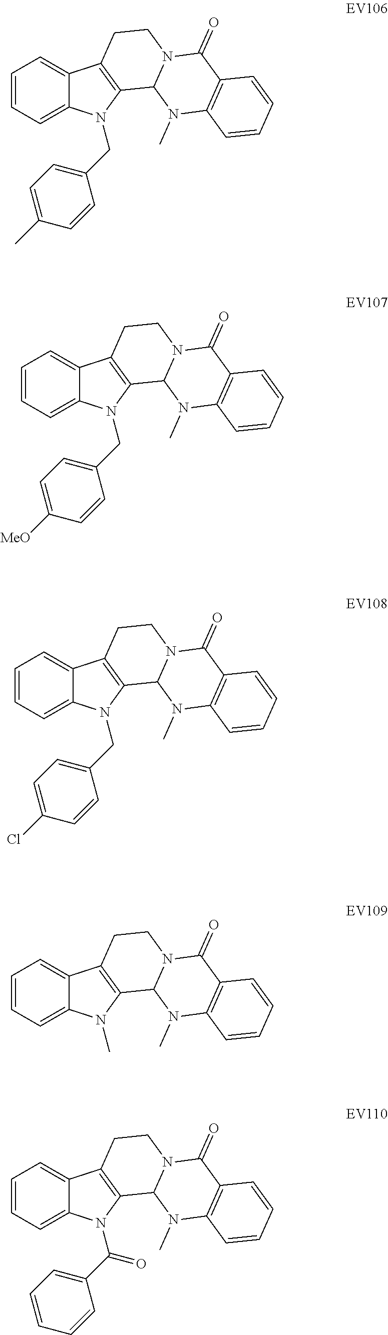 [SMILES: Cc1ccc(Cn2c3c(c4ccccc42)CCN2C(=O)c4ccccc4N(C)C32)cc1 ; COc1ccc(Cn2c3c(c4ccccc42)CCN2C(=O)c4ccccc4N(C)C32)cc1 ; CN1c2ccccc2C(=O)N2CCc3c(n(Cc4ccc(Cl)cc4)c4ccccc34)C21 ; CN1c2ccccc2C(=O)N2CCc3c(n(C)c4ccccc34)C21 ; CN1c2ccccc2C(=O)N2CCc3c(n(C(=O)c4ccccc4)c4ccccc34)C21]

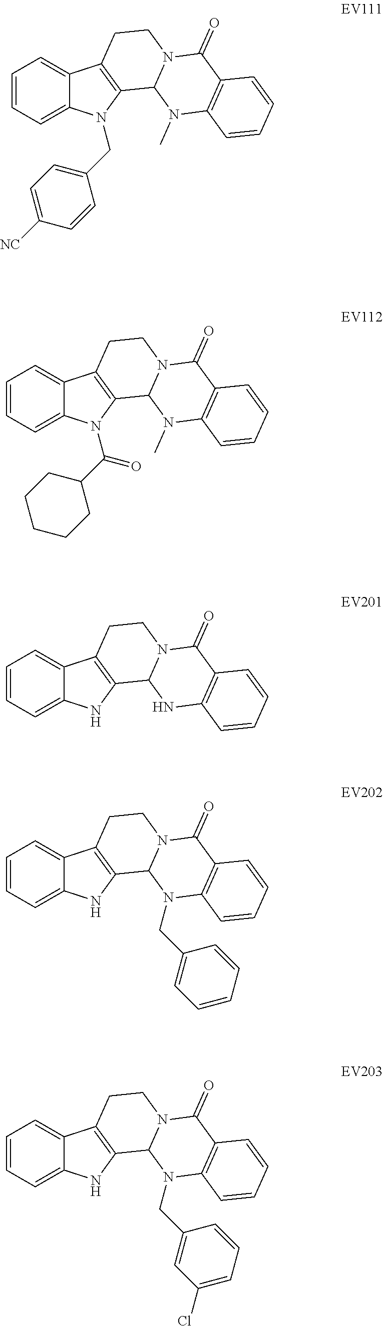 [SMILES: CN1c2ccccc2C(=O)N2CCc3c(n(Cc4ccc(C#N)cc4)c4ccccc34)C21 ; CN1c2ccccc2C(=O)N2CCc3c(n(C(=O)C4CCCCC4)c4ccccc34)C21 ; O=C1c2ccccc2NC2c3[nH]c4ccccc4c3CCN12 ; O=C1c2ccccc2N(Cc2ccccc2)C2c3[nH]c4ccccc4c3CCN12 ; O=C1c2ccccc2N(Cc2cccc(Cl)c2)C2c3[nH]c4ccccc4c3CCN12]

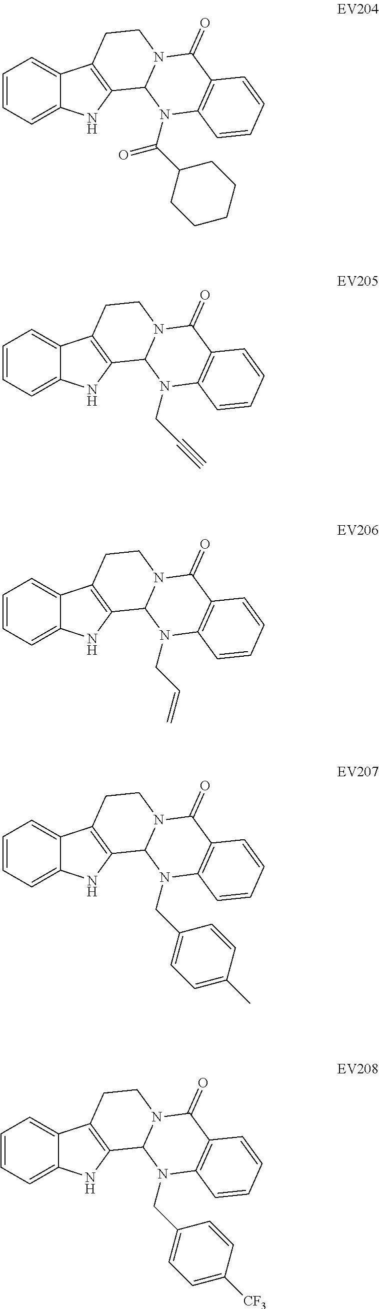 [SMILES: O=C1c2ccccc2N(C(=O)C2CCCCC2)C2c3[nH]c4ccccc4c3CCN12 ; C#CCN1c2ccccc2C(=O)N2CCc3c([nH]c4ccccc34)C21 ; C=CCN1c2ccccc2C(=O)N2CCc3c([nH]c4ccccc34)C21 ; Cc1ccc(CN2c3ccccc3C(=O)N3CCc4c([nH]c5ccccc45)C32)cc1 ; O=C1c2ccccc2N(Cc2ccc(C(F)(F)F)cc2)C2c3[nH]c4ccccc4c3CCN12]

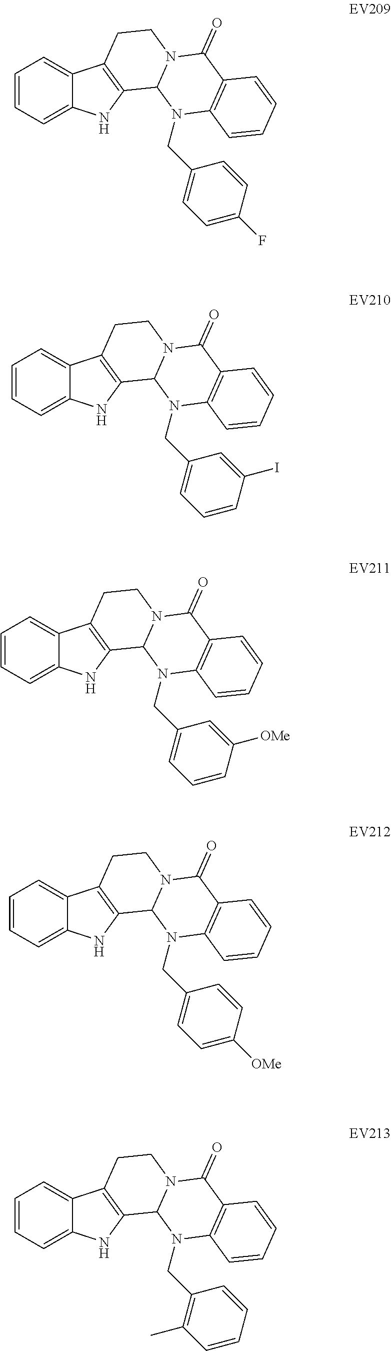 [SMILES: O=C1c2ccccc2N(Cc2ccc(F)cc2)C2c3[nH]c4ccccc4c3CCN12 ; O=C1c2ccccc2N(Cc2cccc(I)c2)C2c3[nH]c4ccccc4c3CCN12 ; COc1cccc(CN2c3ccccc3C(=O)N3CCc4c([nH]c5ccccc45)C32)c1 ; COc1ccc(CN2c3ccccc3C(=O)N3CCc4c([nH]c5ccccc45)C32)cc1 ; Cc1ccccc1CN1c2ccccc2C(=O)N2CCc3c([nH]c4ccccc34)C21]

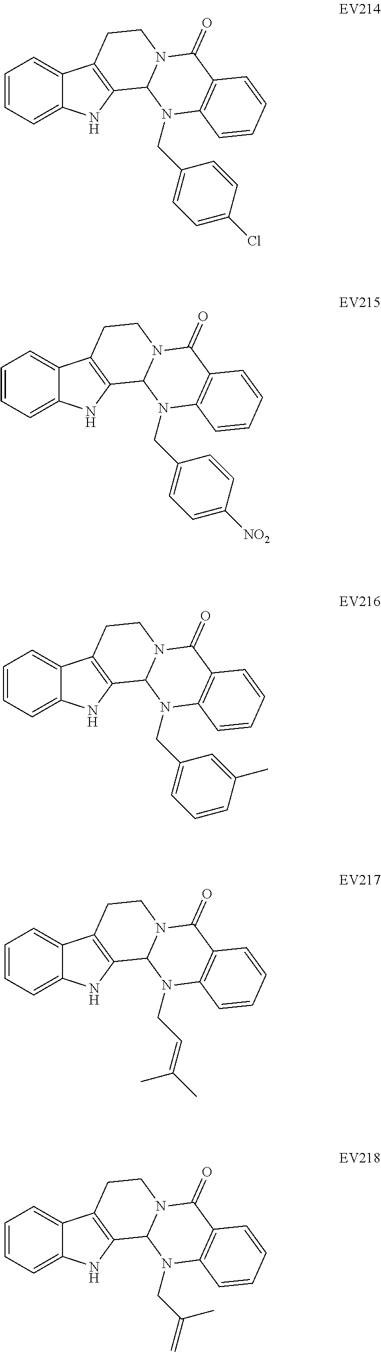 [SMILES: O=C1c2ccccc2N(Cc2ccc(Cl)cc2)C2c3[nH]c4ccccc4c3CCN12 ; O=C1c2ccccc2N(Cc2ccc([N+](=O)[O-])cc2)C2c3[nH]c4ccccc4c3CCN12 ; Cc1cccc(CN2c3ccccc3C(=O)N3CCc4c([nH]c5ccccc45)C32)c1 ; CC(C)=CCN1c2ccccc2C(=O)N2CCc3c([nH]c4ccccc34)C21 ; C=C(C)CN1c2ccccc2C(=O)N2CCc3c([nH]c4ccccc34)C21]

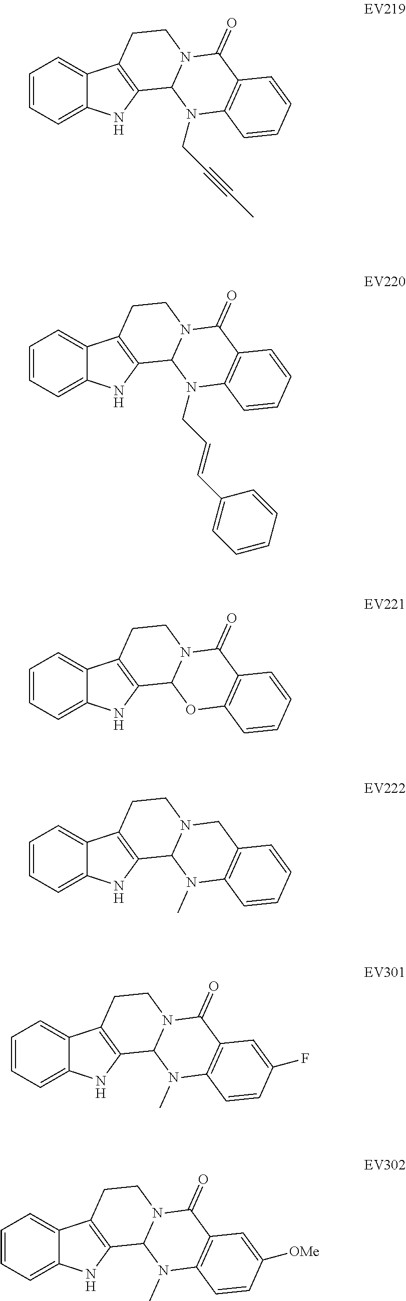 [SMILES: CC#CCN1c2ccccc2C(=O)N2CCc3c([nH]c4ccccc34)C21 ; O=C1c2ccccc2N(C/C=C/c2ccccc2)C2c3[nH]c4ccccc4c3CCN12 ; O=C1c2ccccc2OC2c3[nH]c4ccccc4c3CCN12 ; CN1c2ccccc2CN2CCc3c([nH]c4ccccc34)C21 ; CN1c2ccc(F)cc2C(=O)N2CCc3c([nH]c4ccccc34)C21 ; COc1ccc2c(c1)C(=O)N1CCc3c([nH]c4ccccc34)C1N2C]

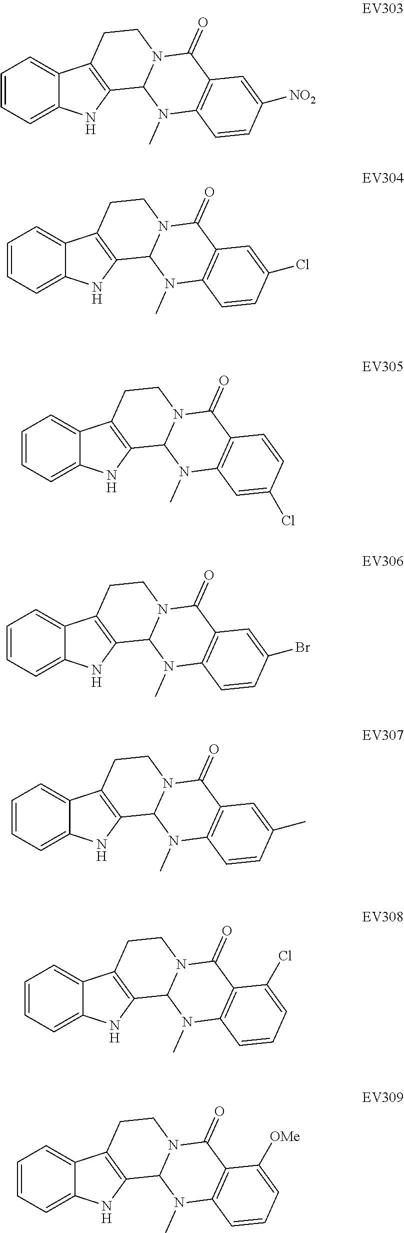

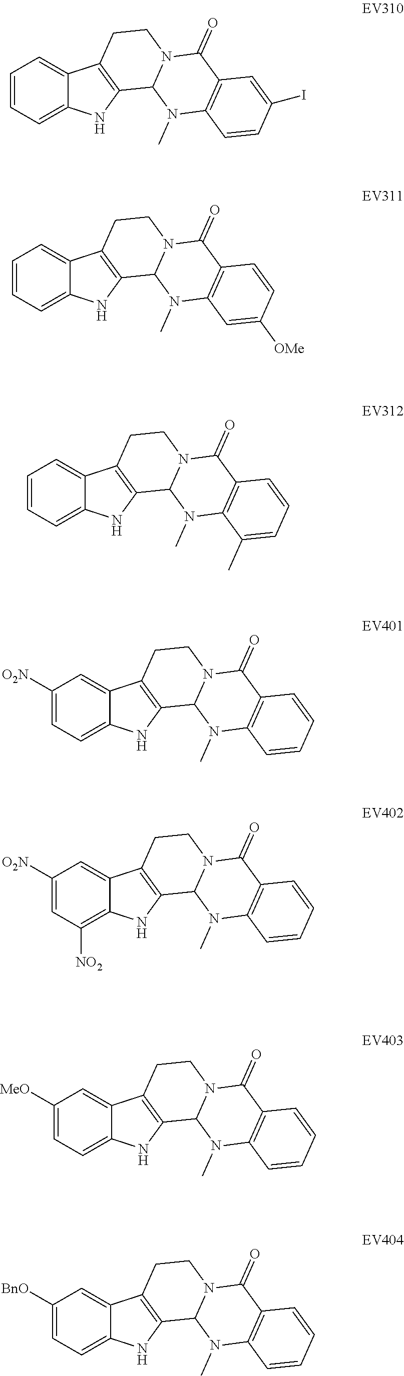

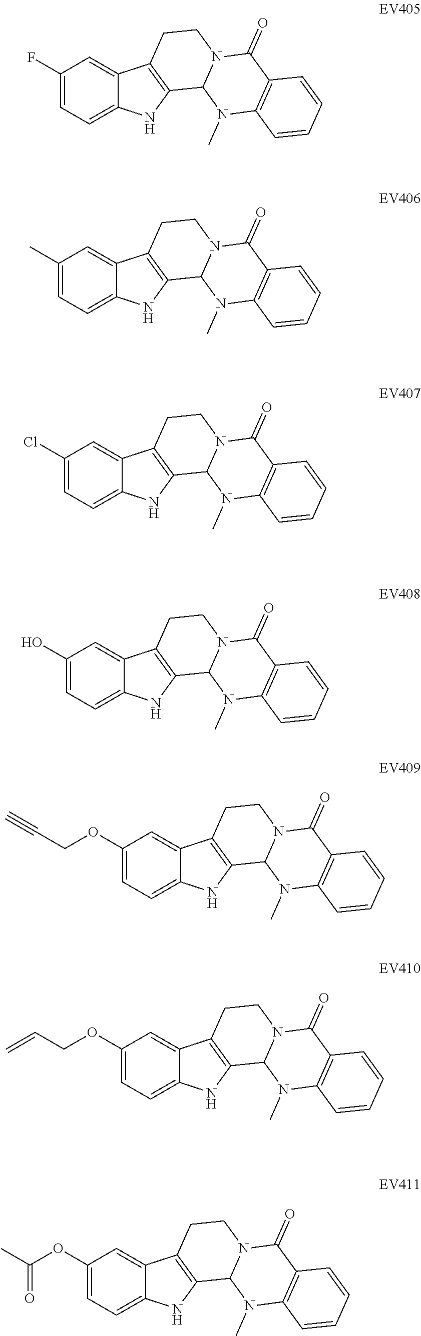

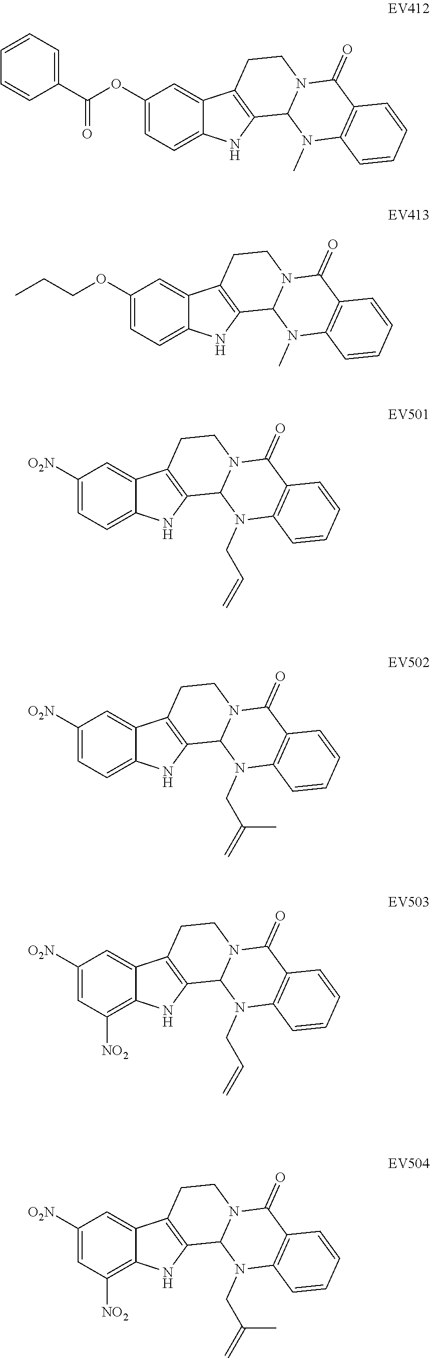 [SMILES: CN1c2ccccc2C(=O)N2CCc3c([nH]c4ccc(OC(=O)c5ccccc5)cc34)C21 ; CCCOc1ccc2[nH]c3c(c2c1)CCN1C(=O)c2ccccc2N(C)C31 ; C=CCN1c2ccccc2C(=O)N2CCc3c([nH]c4ccc([N+](=O)[O-])cc34)C21 ; C=C(C)CN1c2ccccc2C(=O)N2CCc3c([nH]c4ccc([N+](=O)[O-])cc34)C21 ; C=CCN1c2ccccc2C(=O)N2CCc3c([nH]c4c([N+](=O)[O-])cc([N+](=O)[O-])cc34)C21 ; C=C(C)CN1c2ccccc2C(=O)N2CCc3c([nH]c4c([N+](=O)[O-])cc([N+](=O)[O-])cc34)C21]

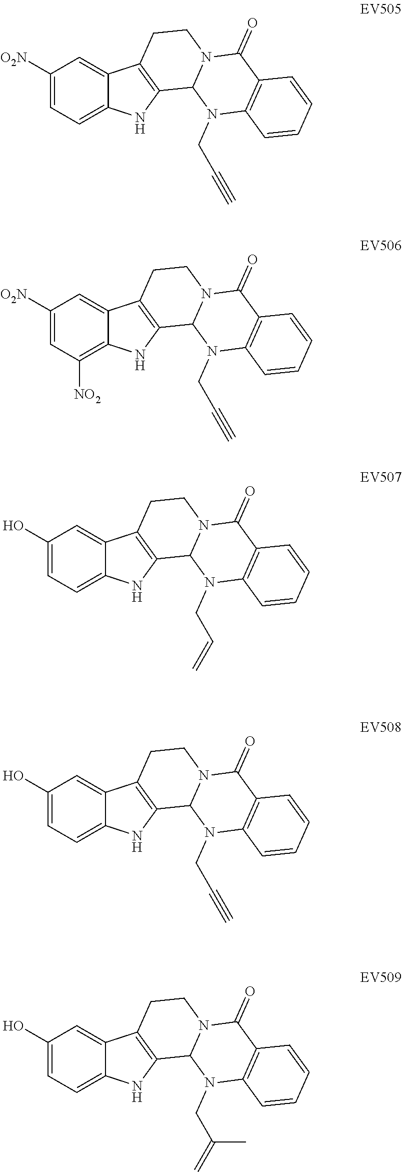 [SMILES: C#CCN1c2ccccc2C(=O)N2CCc3c([nH]c4ccc([N+](=O)[O-])cc34)C21 ; C#CCN1c2ccccc2C(=O)N2CCc3c([nH]c4c([N+](=O)[O-])cc([N+](=O)[O-])cc34)C21 ; C=CCN1c2ccccc2C(=O)N2CCc3c([nH]c4ccc(O)cc34)C21 ; C#CCN1c2ccccc2C(=O)N2CCc3c([nH]c4ccc(O)cc34)C21 ; C=C(C)CN1c2ccccc2C(=O)N2CCc3c([nH]c4ccc(O)cc34)C21]

In yet another embodiment of the present invention, the cancer may be at least one selected from the group consisting of cervical cancer, lung cancer, pancreatic cancer, non-small cell lung cancer, liver cancer, colon cancer, colorectal cancer, bone cancer, skin cancer, head and neck cancer, cutaneous melanoma, intraocular melanoma, uterine cancer, ovarian cancer, rectal cancer, liver cancer, brain tumor, blood cancer, gastric cancer, anal cancer, breast cancer, fallopian tube cancer, endometrial cancer, vaginal cancer, vulvar cancer, Hodgkin's disease, esophageal cancer, small intestine cancer, endocrine gland cancer, thyroid cancer, parathyroid cancer, adrenal cancer, soft tissue sarcoma, urethral cancer, penile cancer, prostate cancer, bladder cancer, kidney cancer, ureteral cancer, renal cell carcinoma, renal pelvis carcinoma, central nervous system (CNS) tumor, primary CNS lymphoma, spinal cord tumor, brain stem glioma and pituitary adenoma, but is not limited thereto.

In yet another embodiment of the present invention, the cancer may be lung cancer, but is not limited thereto.

In yet another embodiment of the present invention, the indoloquinazolidine alkaloid may be capable of reducing at least one selected from the group consisting of the number and volume of tumors, but is not limited thereto.

In yet another embodiment of the present invention, the indoloquinazolidine alkaloid may be capable of suppressing the expression of HSP70 protein, but is not limited thereto.

In yet another embodiment of the present invention, the indoloquinazolidine alkaloid may be capable of suppressing the expression of at least one protein or gene selected from the group consisting of Akt, Src, and MEK, but is not limited thereto.

In yet another embodiment of the present invention, the composition may further comprise at least one more selected from the group consisting of pemetrexed, cisplatin, and paclitaxel, but is not limited thereto.

In yet another embodiment of the present invention, the composition may be for preventing or treating lung cancer that has acquired resistance to an anti-cancer drug, but is not limited thereto.

Furthermore, the present invention provides an anti-cancer adjuvant that enhances the anti-cancer efficacy of an anti-cancer drug, comprising an indoloquinazolidine alkaloid represented by Chemical Formula 1, or a pharmaceutically acceptable salt thereof as an active ingredient.

In one embodiment of the present invention, the anti-cancer drug may be at least one selected from the group consisting of pemetrexed, cisplatin, and paclitaxel, but is not limited thereto.

Furthermore, the present invention provides a method for enhancing or promoting the anti-cancer efficacy of an anti-cancer drug, the method comprising administering an anti-cancer adjuvant comprising an indoloquinazolidine alkaloid represented by Chemical Formula 1 or a pharmaceutically acceptable salt thereof as an active ingredient to a subject in need thereof.

Additionally, the present invention provides a use for enhancing or promoting the anti-cancer efficacy of an anti-cancer drug of a composition comprising an indoloquinazolidine alkaloid represented by Chemical Formula 1 or a pharmaceutically acceptable salt thereof as an active ingredient.

Moreover, the present invention provides a use of an indoloquinazolidine alkaloid represented by Chemical Formula 1 or a pharmaceutically acceptable salt thereof for producing a medicine for enhancing or promoting the anti-cancer efficacy of an anti-cancer drug.

### Advantageous Effects

The indoloquinazolidine alkaloid of the present invention is expected to be widely usable for preventing and treating of various cancers as it inhibits tumor growth, suppresses the expression of HSP70 protein and the colony-forming ability of cancer cells, and not only suppresses tumor growth in heterotransplanted cancer cell lines and patient-derived cancer xenograft mouse models, but also inhibits the growth of drug-resistant cancer cells such as those resistant to pemetrexed, cisplatin, and paclitaxel.

### DESCRIPTION OF THE DRAWINGS

FIG. 1 shows changes in cell viability after treatment with EV501-EV509 100 nM for 3 days in H1299 lung cancer cells, as confirmed by MTT assay.

FIG. 2 shows changes in cell viability after treatment with EV507 at various concentrations for 3 days in cancer cell lines of various origins, as confirmed by MTT assay (in lung cancer cell lines and chemotherapeutic drug-resistant lung cancer cell lines) or by crystal violet assay (in the remaining cell lines).

FIG. 3 shows changes in colony formation ability after treatment with EV507 (50 nM) for 2 weeks in cancer cell lines of various origins.

FIG. 4 shows that the expression of Hsp70 and the expression of Hsp90/Hsp70 client proteins are decreased by EV507 in H1299, H460, A549, and H226B lung cancer cell lines.

FIG. 5 shows that the expression of Hsp70 protein is decreased when EV507 (50 nM) is treated in H1299 lung cancer cells, HCT116 colon cancer cells, and UMSCC38 head and neck cancer cell lines.

FIG. 6 shows that EV507 binds to the full length and N-terminal domain of Hsp70.

FIG. 7 shows the tumor growth inhibitory effect of EV507 on xenograft tumors of lung cancer cell lines or those derived from lung cancer patients transplanted into immune-compromised mice.

FIG. 8 shows the effect of EV507 on increasing mouse survival in a lung metastasis homograft model.

FIG. 9 shows changes in cell viability after treatment with various concentrations (0.5-10 μM) of EV101-EV112 for 3 days in H1299 cells, as confirmed by MTT assay.

FIG. 10 shows changes in cell viability after treatment with various concentrations (0.5-10 μM) of EV200-EV222 for 3 days in H1299 cells, as confirmed by MTT assay.

FIG. 11 shows changes in cell viability after treatment with various concentrations (0.5-10 μM) of EV301-EV312 for 3 days in H1299 cells, as confirmed by MTT assay.

FIG. 12 shows changes in cell viability after treatment with various concentrations (0.5-10 μM) of EV401-EV413 for 3 days in H1299 cells, as confirmed by MTT assay.

FIG. 13 shows the cell survival inhibitory effect of EV206 in various lung cancer cells and chemotherapeutic drug-resistant lung cancer cell lines.

FIG. 14 shows the anchorage-dependent colony formation inhibitory effect of EV206 in various lung cancer cells and chemotherapeutic drug-resistant lung cancer cell lines.

FIG. 15 shows the cell viability inhibitory effect of EV408 in various lung cancer cells and chemotherapeutic drug-resistant lung cancer cell lines.

FIG. 16 shows changes in the ability to form anchorage-dependent colonies by treatment with EV408 in various lung cancer cells and chemotherapeutic drug-resistant lung cancer cell lines.

FIG. 17 shows changes in the expression of Hsp70 and Hsp90/Hsp70 client proteins (Akt, Src, and MEK) after treating H1299 cells with up to 250 nM of evodiamine, EV206, and EV408 each for 2 days, as confirmed by Western blot analysis.

FIG. 18 shows the cancer cell survival inhibitory activity (MTT assay) of evodiamine derivatives EV501 to EV509.

FIG. 19 shows the cancer cell survival inhibitory activity (Crystal violet assay) of evodiamine derivatives EV501, EV504, EV507, EV508, and EV509.

FIG. 20 shows the inhibitory activity of evodiamine derivatives EV501 to EV509 on colony formation in soft agar.

FIG. 21 shows changes in the expression of Hsp70 and Hsp90/Hsp70 client proteins (Akt and Src) by Western blot analysis after treatment with evodiamine derivatives EV501 and EV507.

### BEST MODES OF THE INVENTION

The present invention will now be described in detail.

This invention relates to an indoloquinazolidine alkaloid represented by the following Chemical Formula 1 or a pharmaceutically acceptable salt thereof.

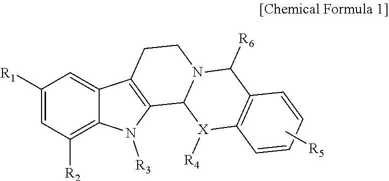

(In the Chemical Formula 1 above,
R₁ and R₂ are each independently hydrogen, substituted or unsubstituted C₁-C₆ alkyl, substituted or unsubstituted C₁-C₆ alkoxy, halogen, nitro (NO₂), amide, —NH-aryl, hydroxy, or —COOR₇,
R₃ is hydrogen, substituted or unsubstituted C₁-C₆ alkyl, —CH₂—R₈, substituted or unsubstituted C₁-C₆ acyl, substituted or unsubstituted aryl, —CO—R₈, or benzyl,
R4 is hydrogen, substituted or unsubstituted C₁-C₆ alkyl, substituted or unsubstituted C₂-C₆ alkenyl, substituted or unsubstituted C₂-C₆ alkynyl, substituted or unsubstituted C₂-C₆ alkylalkenyl, substituted or unsubstituted C₂-C₆ alkylalkynyl, —CH₂—R₈, substituted or unsubstituted aryl, propargyl, substituted or unsubstituted C₁-C₆ acyl, or benzyl,
R₅ is hydrogen, substituted or unsubstituted C₁-C₆ alkyl, halogen, nitro, or substituted or unsubstituted C₁-C₆ alkoxy,
R₆ is hydrogen, oxygen, or substituted or unsubstituted C₁-C₆ alkyl,
R₇ and R₈ are each independently hydrogen, substituted or unsubstituted C₃-C₈ cycloalkyl, substituted or unsubstituted C₁-C₆ alkyl, or substituted or unsubstituted aryl,
X is C, N, O or S.)

Furthermore, the present invention provides a pharmaceutical composition for preventing or treating cancer, which comprises an indoloquinazolidine alkaloid or a pharmaceutically acceptable salt thereof as an active ingredient.

In the present invention, the R₁ and R₂ may each independently be hydrogen, methoxy, butoxy, methyl, hydroxy, F, Cl, nitro,

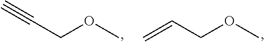

### or —COOR₇,

R₃ may be hydrogen, methyl, ethyl, propyl, butyl, hexyl, benzyl, —CO—R₈ or —CH₂—R₈,
R₄ may be hydrogen, methyl, benzyl, substituted or unsubstituted C₂-C₅ alkenyl, substituted or unsubstituted C₂-C₅ alkynyl, substituted or unsubstituted C₂-C₅ alkylalkenyl, substituted or unsubstituted C₂-C₅ alkylalkynyl, cyclohexanecarbonyl,

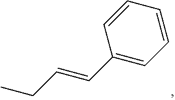

or —CH₂—R₈,
R₅ may be hydrogen, methoxy, nitro, methyl, F, Cl, Br, or I,
R₆ may be hydrogen, or oxygen,
R₇ and R₈ may each independently be hydrogen, substituted or unsubstituted C₃-C₈ cycloalkyl, substituted or unsubstituted C₁-C₆ alkyl, or substituted or unsubstituted aryl, wherein the substituted aryl may be substituted with at least one selected from the group consisting of halogen, CN, C₁-C₆ haloalkyl, C₁-C₃ alkyl, C₁-C₃ alkoxy, and nitro group,
X may be N, or O, but is not limited thereto.

In the present invention, the term “C₁-C₆ alkyl” refers to a monovalent alkyl group of 1 to 6 carbon atoms. This term can exemplify functional groups such as methyl, ethyl, n-propyl, i-propyl, n-butyl, i-butyl, tert-butyl, n-hexyl, etc. The alkyl mentioned in this invention, and any other alkyl portion including substituents, encompasses both linear and branched forms. Substituted C₁-C₆ alkyl means one in which one or more hydrogen atoms have been replaced with another substituent. Although the substituent is not limited, it includes halogen, N, O, S, or substituted or unsubstituted aryl, etc.

In the present invention, the term “halogen” can include fluorine (F), chlorine (Cl), bromine (Br), and iodine (I).

In the present invention, the term “C₁-C₆ alkoxy” refers to —O—R group, where R represents “C₁-C₆ alkyl”. Preferred alkoxy groups can include, for example, methoxy, ethoxy, phenoxy, etc.

In the present invention, the term “aryl” means an unsaturated cyclic aromatic compound of 6 to 20 carbon atoms with a single ring (for example, phenyl) or multiple condensed rings (for example, naphthyl). The aryl can be selected from the group consisting of phenyl, naphthyl, anthryl, and biaryl.

In the present invention, the indoloquinazolidine alkaloid may reduce at least one selected from the group consisting of the number and volume of tumors, but is not limited thereto.

In the present invention, the indoloquinazolidine alkaloid may inhibit the expression of HSP70 protein, but is not limited thereto. The HSP70 (Heat Shock Protein 70) is a type of chaperone involved in the protein folding process for the complete structural formation of proteins. In various cancer cells, HSP70 is overexpressed and mediates the survival of cancer cells and resistance to anti-cancer drugs. The cancer can be prevented or treated by inhibiting HSP70.

In the present invention, the indoloquinazolidine alkaloid may inhibit the expression of one or more proteins or genes selected from the group consisting of Akt, Src, and MEK, but is not limited thereto.

The term “cancer” as used herein refers to a disease involving the regulation of cell death, which results from the overgrowth of cells when the balance of normal cell suicide (apoptosis) is disturbed. In some cases, these abnormal overgrowth cells can invade surrounding tissues and organs to form a mass and cause the destruction or alteration of normal structures in the body, a condition collectively known as cancer.

Generally, a tumor refers to an abnormal growth caused by the autonomous excessive growth of body tissue. Tumors can be classified into benign tumors and malignant tumors. Malignant tumors grow very fast compared to benign tumors, and they threaten life as they invade surrounding tissues and metastasize. Such malignant tumors are commonly referred to as ‘cancer’.

In the present invention, the cancer may be at least one selected from the group consisting of cervical cancer, lung cancer, pancreatic cancer, non-small cell lung cancer, liver cancer, colon cancer, colorectal cancer, bone cancer, skin cancer, head and neck cancer, cutaneous melanoma, intraocular melanoma, uterine cancer, ovarian cancer, rectal cancer, liver cancer, brain tumor, blood cancer, gastric cancer, anal cancer, breast cancer, fallopian tube cancer, endometrial cancer, vaginal cancer, vulvar cancer, Hodgkin's disease, esophageal cancer, small intestine cancer, endocrine gland cancer, thyroid cancer, parathyroid cancer, adrenal cancer, soft tissue sarcoma, urethral cancer, penile cancer, prostate cancer, bladder cancer, kidney cancer, ureteral cancer, renal cell carcinoma, renal pelvis carcinoma, central nervous system (CNS) tumor, primary CNS lymphoma, spinal cord tumor, brain stem glioma and pituitary adenoma, but is not limited thereto.

In the present invention, the cancer may be a drug-resistant cancer, but is not limited thereto. Drug-resistant cancer refers to a situation where drug-resistant cells make up most of the cancer cells. This happens as cells with drug resistance selectively survive and proliferate, resulting in the majority of the cancer mass becoming drug-resistant. The drug may be pemetrexed, cisplatin, or paclitaxel, but is not limited thereto.

In the present invention, the cancer may be lung cancer, specifically it may be small cell lung cancer or non-small cell lung cancer, but is not limited thereto.

In this specification, “lung cancer” refers to a malignant tumor in the lung. The types of primary lung cancer, originating from the lung itself, are divided into non-small cell lung cancer and small cell lung cancer, based on the size and shape of the cancer cells.

In this specification, “non-small cell lung cancer (NSCLC)” refers to lung cancer in which the cancer cells are not small in size. It accounts for 80-85% of lung cancers and includes squamous cell carcinoma, adenocarcinoma, large cell carcinoma, adenosquamous carcinoma, sarcomatoid carcinoma, carcinoid tumor, salivary gland type carcinoma, and unclassified carcinoma. Non-small cell lung cancer may arise due to mutations, which can include EGFR (epidermal growth factor receptor) mutant non-small cell lung cancer, KRAS (v-Ki-ras2 Kirsten rat sarcoma viral oncogene homolog) mutant non-small cell lung cancer, and ALK (Anaplastic Lymphoma Kinase) mutant non-small cell lung cancer, among others.

The present invention may also comprise a pharmaceutically acceptable salt as an active ingredient. In the present invention, the term “pharmaceutically acceptable salt” comprises a salt derived from pharmaceutically acceptable inorganic acids, organic acids, or bases.

Examples of suitable acids include hydrochloric acid, bromic acid, sulfuric acid, nitric acid, perchloric acid, fumaric acid, maleic acid, phosphoric acid, glycolic acid, lactic acid, salicylic acid, succinic acid, toluene-p-sulfonic acid, tartaric acid, acetic acid, citric acid, methanesulfonic acid, formic acid, benzoic acid, malonic acid, gluconic acid, naphthalene-2-sulfonic acid, benzene sulfonic acid, etc. Acid addition salts can be manufactured by conventional methods, for example, by dissolving the compound in an excess of an acid aqueous solution and precipitating this salt using a water-miscible organic solvent such as methanol, ethanol, acetone, or acetonitrile. Acid addition salts can also be prepared by heating an equimolar amount of the compound and an acid in water or alcohol and subsequently evaporating the mixture to dryness, or by aspiration-filtration of the precipitated salt.

Salts derived from suitable bases can include alkali metals such as sodium, potassium, alkaline earth metals such as magnesium, and ammonium, but are not limited thereto. Alkali metal or alkaline earth metal salts, for example, can be obtained by dissolving the compound in an excess of alkali metal hydroxide or alkaline earth metal hydroxide solution, filtering off the insoluble compound salt, and then evaporating and drying the filtrate. Here, it is pharmaceutically suitable to manufacture particularly sodium, potassium, or calcium salts as the metal salts, and the corresponding silver salts can be obtained by reacting an alkali metal or alkaline earth metal salt with a suitable silver salt (for example, silver nitrate).

The content of the compound in the composition of the present invention may be appropriately adjusted depending on the symptoms of a disease, the degree of progression of symptoms, the condition of a patient, and the like, and may range from, for example, 0.0001 wt % to 99.9 wt % or 0.001 wt % to 50 wt % with respect to a total weight of the composition, but the present invention is not limited thereto. The amount ratio is a value based on the amount of dried product from which a solvent is removed.

The pharmaceutical composition according to the present invention may further include a suitable carrier, excipient, and diluent which are commonly used in the preparation of pharmaceutical compositions. The excipient may be, for example, one or more selected from the group consisting of a diluent, a binder, a disintegrant, a lubricant, an adsorbent, a humectant, a film-coating material, and a controlled release additive.

The pharmaceutical composition according to the present invention may be used by being formulated, according to commonly used methods, into a form such as powders, granules, sustained-release-type granules, enteric granules, liquids, eye drops, elixirs, emulsions, suspensions, spirits, troches, aromatic water, lemonades, tablets, sustained-release-type tablets, enteric tablets, sublingual tablets, hard capsules, soft capsules, sustained-release-type capsules, enteric capsules, pills, tinctures, soft extracts, dry extracts, fluid extracts, injections, capsules, perfusates, or a preparation for external use, such as plasters, lotions, pastes, sprays, inhalants, patches, sterile injectable solutions, or aerosols. The preparation for external use may have a formulation such as creams, gels, patches, sprays, ointments, plasters, lotions, liniments, pastes, or cataplasmas.

As the carrier, the excipient, and the diluent that may be included in the pharmaceutical composition according to the present invention, lactose, dextrose, sucrose, oligosaccharides, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil may be used.

For formulation, commonly used diluents or excipients such as fillers, thickeners, binders, wetting agents, disintegrants, and surfactants are used.

As additives of tablets, powders, granules, capsules, pills, and troches according to the present invention, excipients such as corn starch, potato starch, wheat starch, lactose, white sugar, glucose, fructose, D-mannitol, precipitated calcium carbonate, synthetic aluminum silicate, dibasic calcium phosphate, calcium sulfate, sodium chloride, sodium hydrogen carbonate, purified lanolin, microcrystalline cellulose, dextrin, sodium alginate, methyl cellulose, sodium carboxymethylcellulose, kaolin, urea, colloidal silica gel, hydroxypropyl starch, hydroxypropyl methylcellulose (HPMC), HPMC 1928, HPMC 2208, HPMC 2906, HPMC 2910, propylene glycol, casein, calcium lactate, and Primojel®; and binders such as gelatin, Arabic gum, ethanol, agar powder, cellulose acetate phthalate, carboxymethylcellulose, calcium carboxymethylcellulose, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch, hydroxypropyl cellulose, hydroxypropyl methylcellulose, polyvinyl alcohol, and polyvinylpyrrolidone may be used, and disintegrants such as hydroxypropyl methylcellulose, corn starch, agar powder, methylcellulose, bentonite, hydroxypropyl starch, sodium carboxymethylcellulose, sodium alginate, calcium carboxymethylcellulose, calcium citrate, sodium lauryl sulfate, silicic anhydride, 1-hydroxypropylcellulose, dextran, ion-exchange resin, polyvinyl acetate, formaldehyde-treated casein and gelatin, alginic acid, amylose, guar gum, sodium bicarbonate, polyvinylpyrrolidone, calcium phosphate, gelled starch, Arabic gum, amylopectin, pectin, sodium polyphosphate, ethyl cellulose, white sugar, magnesium aluminum silicate, a di-sorbitol solution, and light anhydrous silicic acid; and lubricants such as calcium stearate, magnesium stearate, stearic acid, hydrogenated vegetable oil, talc, lycopodium powder, kaolin, Vaseline, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, higher fatty acids, higher alcohols, silicone oil, paraffin oil, polyethylene glycol fatty acid ether, starch, sodium chloride, sodium acetate, sodium oleate, dl-leucine, and light anhydrous silicic acid may be used.

As additives of liquids according to the present invention, water, dilute hydrochloric acid, dilute sulfuric acid, sodium citrate, monostearic acid sucrose, polyoxyethylene sorbitol fatty acid esters (twin esters), polyoxyethylene monoalkyl ethers, lanolin ethers, lanolin esters, acetic acid, hydrochloric acid, ammonia water, ammonium carbonate, potassium hydroxide, sodium hydroxide, prolamine, polyvinylpyrrolidone, ethylcellulose, and sodium carboxymethylcellulose may be used.

In syrups according to the present invention, a white sugar solution, other sugars or sweeteners, and the like may be used, and as necessary, a fragrance, a colorant, a preservative, a stabilizer, a suspending agent, an emulsifier, a viscous agent, or the like may be used.

In emulsions according to the present invention, purified water may be used, and as necessary, an emulsifier, a preservative, a stabilizer, a fragrance, or the like may be used.

In suspensions according to the present invention, suspending agents such as acacia, tragacanth, methylcellulose, carboxymethylcellulose, sodium carboxymethylcellulose, microcrystalline cellulose, sodium alginate, hydroxypropyl methylcellulose (HPMC), HPMC 1828, HPMC 2906, HPMC 2910, and the like may be used, and as necessary, a surfactant, a preservative, a stabilizer, a colorant, and a fragrance may be used.

Injections according to the present invention may include: solvents such as distilled water for injection, a 0.9% sodium chloride solution, Ringer's solution, a dextrose solution, a dextrose+sodium chloride solution, PEG, lactated Ringer's solution, ethanol, propylene glycol, non-volatile oil-sesame oil, cottonseed oil, peanut oil, soybean oil, corn oil, ethyl oleate, isopropyl myristate, and benzene benzoate; cosolvents such as sodium benzoate, sodium salicylate, sodium acetate, urea, urethane, monoethylacetamide, butazolidine, propylene glycol, the Tween series, amide nicotinate, hexamine, and dimethylacetamide; buffers such as weak acids and salts thereof (acetic acid and sodium acetate), weak bases and salts thereof (ammonia and ammonium acetate), organic compounds, proteins, albumin, peptone, and gums; isotonic agents such as sodium chloride; stabilizers such as sodium bisulfite (NaHSO₃) carbon dioxide gas, sodium metabisulfite (Na₂S₂O₃), sodium sulfite (Na₂SO₃), nitrogen gas (N₂), and ethylenediamine tetraacetic acid; sulfating agents such as 0.1% sodium bisulfide, sodium formaldehyde sulfoxylate, thiourea, disodium ethylenediaminetetraacetate, and acetone sodium bisulfite; a pain relief agent such as benzyl alcohol, chlorobutanol, procaine hydrochloride, glucose, and calcium gluconate; and suspending agents such as sodium CMC, sodium alginate, Tween 80, and aluminum monostearate.

In suppositories according to the present invention, bases such as cacao butter, lanolin, Witepsol, polyethylene glycol, glycerogelatin, methylcellulose, carboxymethylcellulose, a mixture of stearic acid and oleic acid, Subanal, cottonseed oil, peanut oil, palm oil, cacao butter+cholesterol, lecithin, lanette wax, glycerol monostearate, Tween or span, imhausen, monolan(propylene glycol monostearate), glycerin, Adeps solidus, buytyrum Tego-G, cebes Pharma 16, hexalide base 95, cotomar, Hydrokote SP, S-70-XXA, S-70-XX75(S-70-XX95), Hydrokote 25, Hydrokote 711, idropostal, massa estrarium (A, AS, B, C, D, E, I, T), masa-MF, masupol, masupol-15, neosuppostal-N, paramount-B, supposiro OSI, OSIX, A, B, C, D, H, L, suppository base IV types (AB, B, A, BC, BBG, E, BGF, C, D, 299), suppostal (N, Es), Wecoby (W, R, S, M, Fs), and tegester triglyceride matter (TG-95, MA, 57) may be used.

Solid preparations for oral administration include tablets, pills, powders, granules, capsules, and the like, and such solid preparations are formulated by mixing the composition with at least one excipient, e.g., starch, calcium carbonate, sucrose, lactose, gelatin, and the like. In addition to simple excipients, lubricants such as magnesium stearate and talc are also used.

Examples of liquid preparations for oral administration include suspensions, liquids for internal use, emulsions, syrups, and the like, and these liquid preparations may include, in addition to simple commonly used diluents, such as water and liquid paraffin, various types of excipients, for example, a wetting agent, a sweetener, a fragrance, a preservative, and the like. Preparations for parenteral administration include an aqueous sterile solution, a non-aqueous solvent, a suspension, an emulsion, a freeze-dried preparation, and a suppository. Non-limiting examples of the non-aqueous solvent and the suspension include propylene glycol, polyethylene glycol, a vegetable oil such as olive oil, and an injectable ester such as ethyl oleate.

The pharmaceutical composition according to the present invention is administered in a pharmaceutically effective amount. In the present invention, “the pharmaceutically effective amount” refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and factors well known in other medical fields.

The composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this may be easily determined by those of ordinary skill in the art.

The pharmaceutical composition of the present invention may be administered to a subject via various routes. All administration methods can be predicted, and the pharmaceutical composition may be administered via, for example, oral administration, subcutaneous injection, intraperitoneal injection, intravenous injection, intramuscular injection, intrathecal (space around the spinal cord) injection, sublingual administration, administration via the buccal mucosa, intrarectal insertion, intravaginal insertion, ocular administration, intra-aural administration, intranasal administration, inhalation, spraying via the mouth or nose, transdermal administration, percutaneous administration, or the like.

The pharmaceutical composition of the present invention is determined by the type of drug that is the active ingredient, along with other relevant factors such as the disease to be treated, the route of administration, and the patient's age, gender, weight, and severity of the disease.

As used herein, the “subject” refers to a subject in need of treatment of a disease, and more specifically, refers to a mammal such as a human or a non-human primate, a mouse, a rat, a dog, a cat, a horse, and a cow, but the present invention is not limited thereto.

As used herein, the “administration” refers to providing a subject with a predetermined composition of the present invention by using an arbitrary appropriate method.

The term “prevention” as used herein means all actions that inhibit or delay the onset of a target disease. The term “treatment” as used herein means all actions that alleviate or beneficially change a target disease and abnormal metabolic symptoms caused thereby via administration of the pharmaceutical composition according to the present invention. The term “improvement” as used herein means all actions that reduce the degree of parameters related to a target disease, e.g., symptoms via administration of the composition according to the present invention.

Additionally, the present invention provides an anti-cancer adjuvant that enhances the anti-cancer efficacy of an anti-cancer drug, comprising an indoloquinazolidine alkaloid or a pharmaceutically acceptable salt thereof as an active ingredient.

In the present invention, the cancer may be lung cancer, but is not limited thereto.

The anti-cancer adjuvant can enhance the efficacy of the anti-cancer drug. Also, the anti-cancer adjuvant can be administered simultaneously or sequentially with the anti-cancer drug. In case of sequential administration, the anti-cancer adjuvant can be administered before or after the administration of the anti-cancer drug, but is not limited thereto. The administration method can be altered to enhance the anti-cancer efficacy.

The anti-cancer drug may be pemetrexed, cisplatin, or paclitaxel, but is not limited thereto.

The present invention, which can undergo various transformations and have various embodiments, is intended to illustrate specific examples in the drawings and explain in detail in the detailed description. However, this is not intended to limit the present invention to specific forms of implementation, and all transformations, equivalents or substitutes within the concept and technical scope of the present invention should be included. Detailed descriptions of the related known techniques that could obscure the gist of the present invention are omitted in describing the present invention.

### BEST MODES OF THE INVENTION

Hereinafter, preferable examples are presented to aid in the understanding of the present invention. However, these examples are provided merely for easier understanding of the present invention, and the content of the present invention is not limited by these examples.

### Synthesis Example. Method for Synthesizing Evodiamine Derivatives

### Synthesis Example 1. General Method for the Synthesis of Evodiamine (1) and Intermediates

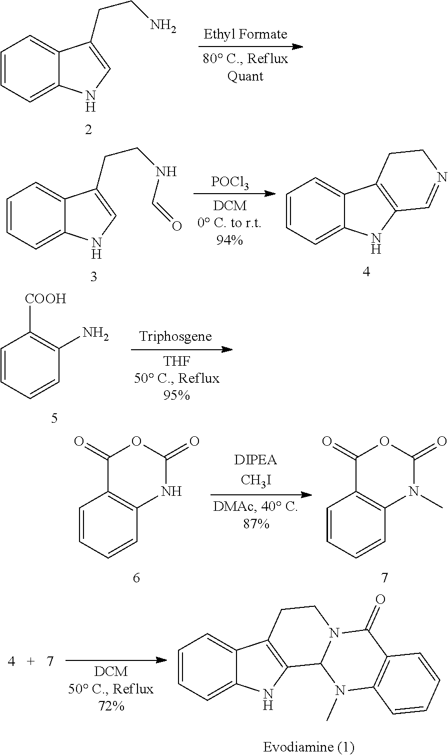

### N-formyltryptamine (3)

:Tryptamine (2) (1.00 g, 6.24 mmol), and ethyl formate (2.3 g, 31.210 mmol) were heated under Ar substitution at 80° C. with stirring. After the reaction was completed, the excess ethyl formate was removed by vacuum concentration, and the resulting mixture was purified by column chromatography (silica gel, MC:MeOH=20:1˜10:1) to obtain a brown oil-form compound 3 (1.2 g, 100% yield).

¹H-NMR (400 MHz, CDCl₃) δ 8.13 (br s, 2H), 7.61 (d, J=8.3 Hz, 1H), 7.38-7.40 (m, 1H), 7.23 (td, J=7.6, 1.4 Hz, 1H), 7.14 (t, J=7.6 Hz, 1H), 7.06 (s, 1H), 5.58 (br s, 1H), 3.64-3.69 (m, 2H), 3.01 (t, J=6.9 Hz, 2H); ¹³C-NMR (100 MHz, CDCl₃) b 161.5, 136.5, 127.3, 122.4, 122.2, 119.5, 118.7, 112.4, 111.5, 42.1, 38.4, 27.4, 25.2; HRMS (FAB) m/z calcd for C₁₁H₁₂N₂O [M+H]⁺: 188.0950, found 188.0955.

### 3,4-dihydro-β-carboline (4)

:N-formyltryptamine (3) (1.1 g, 5.920 mmol) was dissolved in CH₂Cl₂ (15.0 mL), then POCl₃ (1.7 mL, 17.770 mmol) was added slowly at 0° C. After stirring at 0° C. for 2 hours, it was stirred at room temperature for another 2 hours. After the reaction was complete, excess POCl₃ and CH₂Cl₂ were removed by vacuum concentration, and the resulting mixture was added with 1 M HCl (100 mL) and washed once with CH₂Cl₂ (1×30 mL). The washed aqueous phase was adjusted to pH=10 with 1 M NaOH at 0° C., and then extracted three times with CH₂Cl₂ (3×30 mL). The obtained organic phase was dried and filtered through MgSO₄, followed by vacuum concentration. Et₂O was added to the resulting mixture to precipitate the solid, which was then filtered to obtain a yellow solid compound 4 (4.0 g, 64% yield).

m.p.: 99-101° C.; ¹H-NMR (400 MHz, DMSO-D₆) δ 11.32 (s, 1H), 8.36 (t, J=2.3 Hz, 1H), 7.55 (d, J=8.2 Hz, 1H), 7.41 (d, J=8.2 Hz, 1H), 7.17-7.21 (m, 1H), 7.04 (td, J=7.5, 0.9 Hz, 1H), 3.78 (td, J=8.7, 2.1 Hz, 2H), 2.80 (t, J=8.7 Hz, 2H); ¹³C-NMR (100 MHz, DMSO-D₆) δ 151.6, 136.7, 128.4, 124.8, 123.7, 119.7, 119.6, 113.8, 112.5, 48.1, 18.7; HRMS (FAB) m/z calcd for C₁₁H₁₁N₂ [M+H]⁺: 171.0917, found 171.0921.

### Isatoic Anhydride (6)

:Anthranilic acid (5) (1.1 g, 8.090 mmol) was dissolved by adding THF (15.0 mL) at room temperature and then triphosgene (7.2 g, 24.280 mmol) was added. After that, the reaction temperature was raised to 50° C. and stirred for 3 hours. After the reaction was complete, ice water (50 mL) was added to the mixture and then vacuum filtered to obtain a white solid compound 6 (1.3 g, 95% yield).

m.p.: 164-165° C.; ¹H-NMR (400 MHz, DMSO-D₆) δ 11.73 (s, 1H), 7.91 (dd, J=7.8, 1.4 Hz, 1H), 7.72-7.76 (m, 1H), 7.23-7.27 (m, 1H), 7.15 (d, J=7.8 Hz, 1H); ¹³C-NMR (100 MHz, DMSO-D₆) δ 159.9, 147.1, 141.4, 137.00, 129.00, 123.5, 115.4, 110.3; HRMS (FAB) m/z calcd for C₈H₅NO₃ [M+H]⁺: 164.0269, found 164.0271.

### N-methylisatoic anhydride (7)

:Isatoic anhydride (6) (200.0 mg, 1.226 mmol) was dissolved in DMAc (3.0 mL), then DIPEA (0.64 mL, 3.678 mmol) and 1-iodomethane (522.1 mg, 3.678 mmol) were slowly added and stirred overnight at 40° C. Afterwards, H₂O was added to the mixture and after the reaction was complete, the mixture was extracted several times with CH₂Cl₂. The collected organic phase was dried and filtered through MgSO₄, followed by vacuum concentration. Hexane was added to the resulting mixture to precipitate the solid, which was then filtered to obtain a solid compound 7 (1.7 g, 68% yield).

m.p.: 106-107° C.; ¹H-NMR (400 MHz, DMSO-d₆) δ 8.01 (dd, J=7.8, 1.8 Hz, 1H), 7.86 (td, J=7.9, 1.5 Hz, 1H), 7.45 (d, J=8.3 Hz, 1H), 7.34 (t, J=7.6 Hz, 1H), 3.47 (s, 3H); ¹³C-NMR (100 MHz, DMSO-d₆) δ 159.0, 147.8, 142.2, 137.2, 129.3, 123.6, 114.9, 111.6, 31.7; LRMS (FAB) m/z, 334 ([M+H]⁺).

### Evodiamine (1)

:3,4-dihydro-β-carboline (4) (50.0 mg, 0.290 mmol) and N-methylisatoic anhydride (7) (46.1 mg, 0.260 mmol) were dissolved in CH₂Cl₂ (0.7 mL), then stirred under Ar substitution at 50° C. for 6 hours. The formed solid was filtered to obtain a yellow solid compound 1 (67.2 mg, 85% yield).

m.p.: 302-303° C.; ¹H-NMR (400 MHz, DMSO-D₆) δ 11.07 (s, 1H), 7.79 (dd, J=7.6, 1.6 Hz, 1H), 7.46-7.50 (m, 2H), 7.36 (d, J=7.8 Hz, 1H), 6.94-7.13 (m, 4H), 6.13 (s, 1H), 4.61-4.65 (m, 1H), 3.17-3.24 (m, 1H), 2.90-2.95 (m, 1H), 2.88 (s, 3H), 2.79 (dd, J=15.4, 4.8 Hz, 1H); ¹³C-NMR (100 MHz, DMSO-D₆) δ 164.3, 148.8, 136.5, 133.5, 130.6, 128.0, 126.00, 121.9, 120.3, 119.3, 118.9, 118.2, 117.5, 111.7, 111.5, 69.8, 40.9, 36.5, 19.5; HRMS (FAB) m/z calcd for C₁₉H₁₈N₃O [M+H]⁺: 304.1444, found 304.1434.

### Synthetic Example 2. Synthesis of B-Ring Evodiamine Derivatives (EV101˜112)

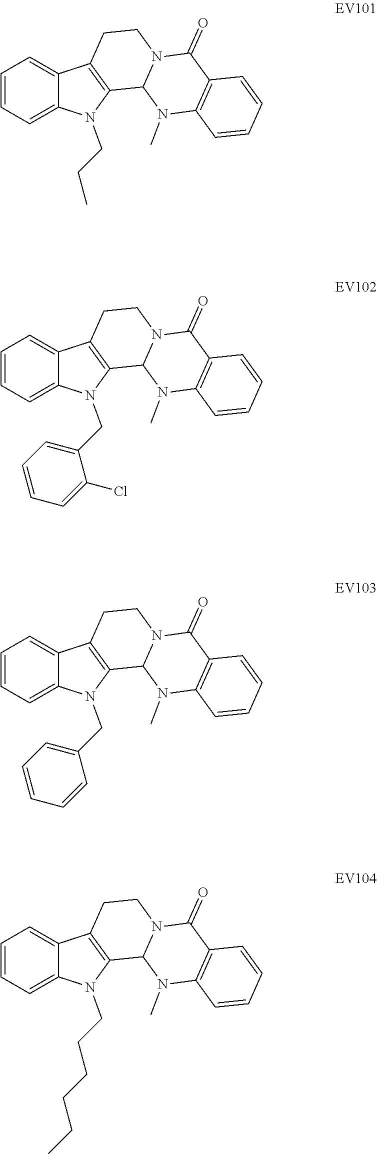 [SMILES: CCCn1c2c(c3ccccc31)CCN1C(=O)c3ccccc3N(C)C21 ; CN1c2ccccc2C(=O)N2CCc3c(n(Cc4ccccc4Cl)c4ccccc34)C21 ; CN1c2ccccc2C(=O)N2CCc3c(n(Cc4ccccc4)c4ccccc34)C21 ; CCCCCCn1c2c(c3ccccc31)CCN1C(=O)c3ccccc3N(C)C21]

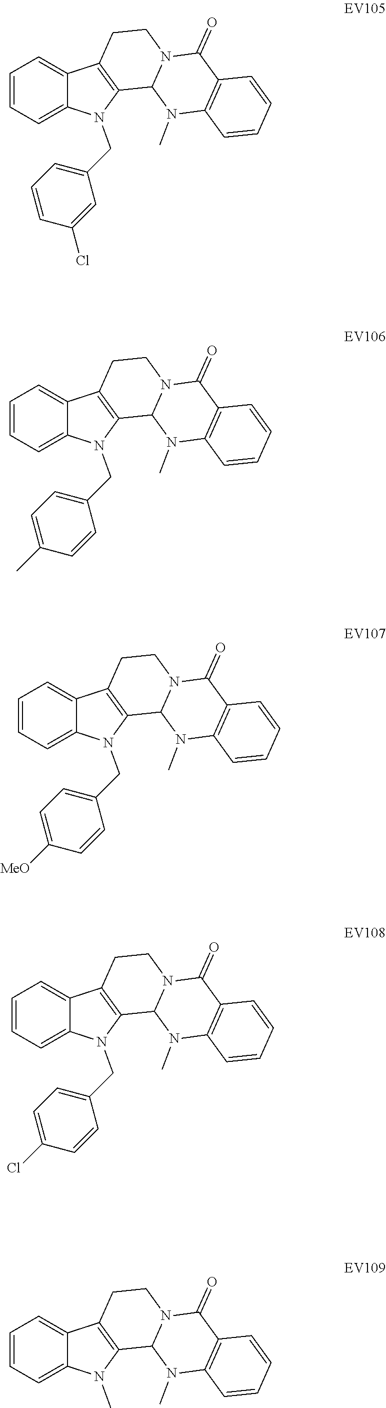 [SMILES: CN1c2ccccc2C(=O)N2CCc3c(n(Cc4cccc(Cl)c4)c4ccccc34)C21 ; Cc1ccc(Cn2c3c(c4ccccc42)CCN2C(=O)c4ccccc4N(C)C32)cc1 ; COc1ccc(Cn2c3c(c4ccccc42)CCN2C(=O)c4ccccc4N(C)C32)cc1 ; CN1c2ccccc2C(=O)N2CCc3c(n(Cc4ccc(Cl)cc4)c4ccccc34)C21 ; CN1c2ccccc2C(=O)N2CCc3c(n(C)c4ccccc34)C21]

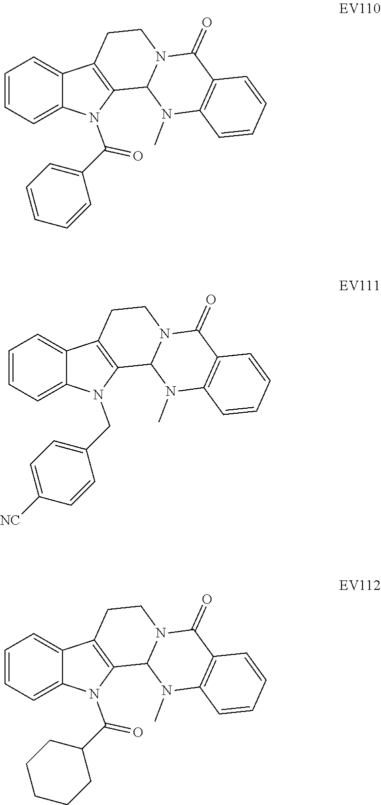 [SMILES: CN1c2ccccc2C(=O)N2CCc3c(n(C(=O)c4ccccc4)c4ccccc34)C21 ; CN1c2ccccc2C(=O)N2CCc3c(n(Cc4ccc(C#N)cc4)c4ccccc34)C21 ; CN1c2ccccc2C(=O)N2CCc3c(n(C(=O)C4CCCCC4)c4ccccc34)C21]

### Method for Synthesis of EV101˜112 Derivatives

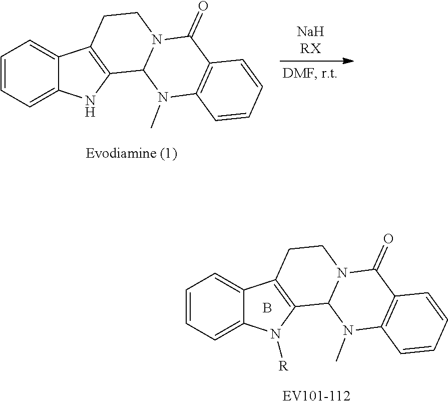

### General Conditions for N-alkylation

:Evodiamine (1) (1.0 equiv.) was dissolved in DMF (0.1 M), then NaH (2.0 equiv.) and alkyl halide (2.0 equiv.) were added in sequence and stirred overnight at room temperature. Afterwards, H₂O (15 mL) was added to the reaction mixture to terminate the reaction, and the aqueous phase was extracted three times with ethyl acetate (3×45 mL). The obtained organic phase was dried and filtered through MgSO₄, followed by vacuum concentration. The resulting mixture was purified by column chromatography (silica gel, hexane:ethyl acetate=3:1˜2:1) to obtain a solid compound EV101-112.

### EV101

:Following the general conditions of the N-alkylation, a yellow solid EV101 (715.5 mg, 72% yield) was obtained using Evodiamine (1) (872.7 mg, 2.877 mmol) and 1-chloropropane (451.9 mg, 5.754 mmol).

m.p.: 168-170° C.; ¹H-NMR (400 MHz, DMSO-d₆) δ 7.96 (d, J=7.8 Hz, 1H), 7.73-7.76 (m, 2H), 7.57 (t, J=7.5 Hz, 1H), 7.43 (t, J=8.7 Hz, 2H), 7.13-7.17 (m, 1H), 7.01 (t, J=7.6 Hz, 1H), 4.73-4.80 (m, 1H), 4.43-4.51 (m, 1H), 4.36 (dd, J=12.6, 4.8 Hz, 1H), 3.77 (q, J=5.7 Hz, 1H), 3.37-3.43 (m, 1H), 2.72-2.85 (m, 2H), 1.92 (d, J=5.5 Hz, 3H), 1.78-1.88 (m, 1H), 1.41-1.54 (m, 1H), 1.05 (t, J=7.4 Hz, 3H); ¹³C-NMR (100 MHz, DMSO-d₆) δ 167.2, 144.9, 136.5, 134.9, 132.5, 132.0, 129.6, 125.7, 124.5, 123.1, 122.2, 119.1, 118.8, 110.4, 109.9, 80.1, 46.4, 34.6, 27.9, 22.7, 21.7, 10.8; LRMS (FAB) m/z, 346 ([M+H]⁺).

### EV102

:Following the general conditions of the N-alkylation, a pale yellow solid EV102 (412.2 mg, 97.4% yield) was obtained using Evodiamine (1) (300.0 mg, 0.989 mmol) and 2-chlorobenzyl chloride (318.5 mg, 1.978 mmol).

m.p.: 242-244° C.; ¹H-NMR (400 MHz, DMSO-d₆) δ 8.17 (d, J=6.9 Hz, 1H), 7.87 (d, J=8.3 Hz, 2H), 7.53-7.71 (m, 6H), 7.13 (t, J=7.6 Hz, 1H), 7.01 (t, J=7.8 Hz, 1H), 6.46 (d, J=8.3 Hz, 1H), 5.03 (q, J=5.5 Hz, 1H), 4.49-4.56 (m, 1H), 3.43 (dd, J=13.6, 5.7 Hz, 1H), 3.28 (d, J=13.3 Hz, 1H), 3.01-3.08 (m, 1H), 2.81-2.85 (m, 1H), 2.52-2.57 (m, 1H), 1.88 (s, 3H); ¹³C-NMR (100 MHz, DMSO-d₆) δ 169.6, 143.2, 136.6, 132.1, 130.4, 130.2, 129.7, 128.7, 128.6, 124.6, 123.8, 123.00, 121.9, 119.6, 114.1, 112.9, 76.4, 58.8, 35.1, 23.35; LRMS (FAB) m/z, 428 ([M+H]⁺).

### EV103

:Following the general conditions of the N-alkylation, a pale yellow solid EV103 (285.6 mg, 73% yield) was obtained using Evodiamine (1) (300.0 mg, 0.989 mmol) and benzyl bromide (338.3 mg, 1.978 mmol).

m.p.: 190-192° C.; ¹H-NMR (400 MHz, DMSO-d₆) δ 7.69 (d, J=7.4 Hz, 1H), 7.63 (d, J=7.8 Hz, 1H), 7.48 (q, J=2.8 Hz, 1H), 7.43 (t, J=7.4 Hz, 1H), 7.23-7.27 (m, 1H), 7.17-7.21 (m, 3H), 6.93-7.03 (m, 3H), 6.85-6.87 (m, 2H), 6.23 (d, J=17.0 Hz, 1H), 5.91 (d, J=17.0 Hz, 1H), 4.41 (dd, J=13.1, 3.4 Hz, 1H), 3.90 (q, J=5.7 Hz, 1H), 3.46-3.53 (m, 1H), 2.79-2.90 (m, 2H), 1.89 (d, J=5.5 Hz, 3H); ¹³C-NMR (100 MHz, DMSO-d₆) b 167.3, 144.3, 138.3, 136.9, 135.6, 132.2, 131.6, 129.3, 128.2, 126.7, 126.0, 126.0, 124.8, 122.9, 122.4, 119.4, 118.9, 110.8, 110.4, 79.9, 48.7, 34.7, 27.9, 21.9; LRMS (FAB) m/z, 394 ([M+H]⁺).

### EV104

:Following the general conditions of the N-alkylation, a pale yellow solid EV104 (383 mg, 100% yield) was obtained using Evodiamine (1) (300.0 mg, 0.989 mmol) and 1-iodohexane (419.4 mg, 1.978 mmol).

m.p.: 66-68° C.; ¹H-NMR (400 MHz, DMSO-d₆) δ 7.95 (d, J=7.4 Hz, 1H), 7.71-7.75 (m, 2H), 7.58 (t, J=7.4 Hz, 1H), 7.44 (d, J=7.8 Hz, 1H), 7.40 (d, J=8.3 Hz, 1H), 7.14-7.18 (m, 1H), 7.01 (t, J=7.1 Hz, 1H), 4.73-4.79 (m, 1H), 4.50-4.58 (m, 1H), 4.36 (dd, J=12.6, 4.8 Hz, 1H), 3.78 (q, J=5.5 Hz, 1H), 3.40 (td, J=12.1, 4.9 Hz, 1H), 2.71-2.84 (m, 2H), 1.91 (d, J=6.0 Hz, 3H), 1.71-1.83 (m, 1H), 1.50 (s, 3H), 1.34 (d, J=3.2 Hz, 4H), 0.91 (t, J=6.9 Hz, 3H); ¹³C-NMR (100 MHz, DMSO-d₆) δ 167.2, 144.9, 136.5, 134.8, 132.5, 132.00, 129.6, 125.8, 124.5, 123.1, 122.2, 119.1, 118.6, 110.3, 110.0, 80.1, 45.0, 34.6, 31.0, 29.3, 27.7, 25.8, 22.0, 21.7, 13.9; LRMS (FAB) m/z, 388 ([M+H]⁺).

### EV105

:Following the general conditions of the N-alkylation, a yellow solid EV105 (385.5 mg, 91% yield) was obtained using Evodiamine (1) (300.0 mg, 0.989 mmol) and 3-chlorobenzyl chloride (630.0 mg, 1.978 mmol).

m.p.: 168-170° C.; ¹H-NMR (400 MHz, DMSO-d₆) δ 7.90 (d, J=7.8 Hz, 1H), 7.63 (d, J=7.8 Hz, 1H), 7.52 (dd, J=13.8, 7.8 Hz, 2H), 7.28-7.32 (m, 2H), 7.21 (t, J=7.4 Hz, 4H), 7.12 (t, J=7.4 Hz, 1H), 7.04 (s, 1H), 6.05 (s, 1H), 5.57 (q, J=17.3 Hz, 2H), 4.66 (dd, J=12.9, 4.1 Hz, 1H), 3.11-3.17 (m, 1H), 3.01 (d, J=14.2 Hz, 1H), 2.83 (t, J=10.6 Hz, 1H), 2.26 (s, 3H); ¹³C-NMR (100 MHz, DMSO-d₆) δ 163.6, 140.8, 137.2, 133.1, 132.9, 130.3, 128.0, 127.1, 126.7, 125.4, 125.4, 123.6, 122.7, 119.6, 118.9, 113.0, 110.3, 46.1, 19.8; LRMS (FAB) m/z, 428 ([M+H]⁺).

### EV106

:Following the general conditions of the N-alkylation, a yellow solid EV106 (371.6 mg, 92% yield) was obtained using Evodiamine (1) (300.0 mg, 0.989 mmol) and 4-methylbenzyl bromide (183.0 mg, 0.989 mmol).

m.p.: 158-160° C.; ¹H-NMR (400 MHz, DMSO-d₆) δ 7.90 (d, J=7.8 Hz, 1H), 7.61 (d, J=8.0 Hz, 1H), 7.54 (t, J=7.6 Hz, 1H), 7.48 (d, J=7.8 Hz, 1H), 7.17-7.26 (m, 3H), 7.04-7.12 (m, 3H), 6.99 (d, J=8.3 Hz, 2H), 5.98 (s, 1H), 5.52 (d, J=3.4 Hz, 2H), 4.66 (dd, J=12.9, 3.7 Hz, 1H), 3.13 (td, J=12.3, 3.8 Hz, 1H), 3.01 (d, J=14.7 Hz, 1H), 2.78-2.85 (m, 1H), 2.34 (s, 3H), 2.21 (s, 3H); ¹³C-NMR (100 MHz, DMSO-d₆) δ 163.5, 137.3, 136.3, 135.0, 133.0, 129.0, 128.0, 126.6, 125.4, 123.5, 122.8, 122.4, 119.4, 118.8, 112.8, 110.4, 46.4, 20.6, 19.8; HRMS (FAB) m/z calcd for C₂₇H₂₆N₃O [M+H]⁺ 408.2076, found 408.2078.

### EV107

:Following the general conditions of the N-alkylation, a white solid EV107 (282.0 mg, 70% yield) was obtained using Evodiamine (1) (300.0 mg, 0.989 mmol) and 4-methoxybenzyl bromide (154.9 mg, 0.989 mmol).

m.p.: 174-176° C.; ¹H-NMR (400 MHz, DMSO-d₆) δ 7.90 (d, J=7.8 Hz, 1H), 7.61 (d, J=7.8 Hz, 1H), 7.50-7.56 (m, 2H), 7.17-7.27 (m, 3H), 7.05-7.12 (m, 3H), 6.81 (d, J=8.7 Hz, 2H), 6.01 (s, 1H), 5.49 (d, J=3.2 Hz, 2H), 4.66 (dd, J=12.9, 4.1 Hz, 1H), 3.66 (s, 3H), 3.12 (td, J=12.2, 3.5 Hz, 1H), 3.00 (d, J=14.7 Hz, 1H), 2.78-2.85 (m, 1H), 2.33 (s, 3H); ¹³C-NMR (100 MHz, DMSO-d₆) δ 163.6, 158.4, 137.2, 133.0, 129.9, 128.1, 125.4, 122.4, 119.4, 118.8, 113.8, 112.7, 110.5, 55.0, 46.1, 19.8; LRMS (FAB) m/z, 424 ([M+H]⁺).

### EV108

:Following the general conditions of the N-alkylation, a yellow solid EV108 (128.4 mg, 30% yield) was obtained using Evodiamine (1) (300.0 mg, 0.989 mmol) and 4-chlorobenzyl chloride (159.3 mg, 0.989 mmol).

m.p.: 202-204° C.; ¹H-NMR (400 MHz, DMSO-d₆) δ 7.86-7.91 (m, 1H), 7.62 (d, J=7.8 Hz, 1H), 7.47-7.55 (m, 2H), 7.31 (t, J=8.3 Hz, 2H), 7.20 (t, J=7.1 Hz, 3H), 7.13 (q, J=7.5 Hz, 3H), 6.02 (s, 1H), 5.56 (d, J=5.5 Hz, 2H), 4.66 (dd, J=12.9, 4.1 Hz, 1H), 3.14 (td, J=12.3, 3.8 Hz, 1H), 3.01 (d, J=14.7 Hz, 1H), 2.80-2.85 (m, 1H), 2.29 (s, 3H); ¹³C-NMR (100 MHz, DMSO-d₆) δ 163.6, 137.2, 133.0, 131.7, 128.5, 128.4, 128.0, 125.4, 122.6, 119.5, 118.9, 113.0, 110.3, 54.9, 46.0, 19.8; HRMS (FAB) m/z calcd for C₂₆H₂₃ClN₃O [M+H]⁺: 428.1530, found 428.1535.

### EV109

:Following the general conditions of the N-alkylation, a yellow solid EV109 (213.4 mg, 68% yield) was obtained using Evodiamine (1) (300.0 mg, 0.989 mmol) and 1-iodomethane (140.4 mg, 0.989 mmol).

m.p.: 176-178° C.; ¹H-NMR (400 MHz, DMSO-d₆) δ 8.13 (d, J=7.9 Hz, 1H), 7.70-7.74 (m, 2H), 7.56 (t, J=7.0 Hz, 1H), 7.43 (dd, J=13.4, 7.9 Hz, 2H), 7.17 (td, J=7.6, 1.2 Hz, 1H), 7.02 (t, J=7.6 Hz, 1H), 4.36-4.40 (m, 1H), 4.19 (s, 3H), 3.79 (q, J=5.7 Hz, 1H), 3.37-3.44 (m, 1H), 2.72-2.83 (m, 2H), 1.93 (d, J=6.1 Hz, 3H); ¹³C-NMR (100 MHz, DMSO-d₆) δ 167.3, 145.2, 137.4, 135.0, 132.2, 132.2, 129.4, 125.3, 124.7, 123.1, 122.3, 119.1, 118.7, 109.9, 109.7, 79.7, 34.8, 32.5, 27.8, 21.7; LRMS (FAB) m/z, 320 ([M+H]⁺).

### EV110

:Following the general conditions of the N-alkylation, a pale yellow solid EV110 (315.8 mg, 78.4% yield) was obtained using Evodiamine (1) (300.0 mg, 0.989 mmol) and benzoyl chloride (278.0 mg, 1.978 mmol).

m.p.: 233-235° C.; ¹H-NMR (400 MHz, DMSO-d₆) δ 7.79 (t, J=7.0 Hz, 3H), 7.71 (t, J=3.1 Hz, 1H), 7.62 (t, J=7.3 Hz, 1H), 7.51 (t, J=7.6 Hz, 2H), 7.41-7.45 (m, 1H), 7.27-7.32 (m, 3H), 7.12 (t, J=7.6 Hz, 1H), 7.03 (d, J=7.9 Hz, 1H), 5.66 (s, 1H), 4.66 (dd, J=12.8, 4.9 Hz, 1H), 3.24 (td, J=12.2, 3.7 Hz, 1H), 3.05 (d, J=14.7 Hz, 1H), 2.82-2.89 (m, 1H), 2.27 (s, 3H); ¹³C-NMR (100 MHz, DMSO-d₆) δ 168.4, 163.4, 150.1, 137.1, 134.6, 133.1, 129.3, 128.8, 127.9, 127.2, 125.2, 123.5, 123.0, 122.7, 122.4, 121.1, 119.6, 114.1, 67.7, 37.7, 36.4, 20.0; LRMS (FAB) m/z, 334 ([M+H]⁺), 307, 273.

### EV111

:Following the general conditions of the N-alkylation, a pale yellow solid EV111 (334.5 mg, 81% yield) was obtained using Evodiamine (1) (300.0 mg, 0.989 mmol) and 4-cyanobenzyl bromide (387.7 mg, 1.978 mmol).

m.p.: 207-209° C.; ¹H-NMR (400 MHz, DMSO-d₆) δ 7.89 (d, J=7.3 Hz, 1H), 7.74 (d, J=7.9 Hz, 2H), 7.64 (d, J=7.9 Hz, 1H), 7.47-7.51 (m, 2H), 7.11-7.25 (m, 6H), 6.03 (s, 1H), 5.66 (dd, J=28.4, 17.4 Hz, 2H), 4.66 (dd, J=12.8, 3.7 Hz, 1H), 3.12-3.18 (m, 1H), 3.01 (d, J=14.7 Hz, 1H), 2.85 (d, J=9.8 Hz, 1H), 2.25 (s, 3H); ¹³C-NMR (100 MHz, DMSO-d₆) δ 163.6, 144.1, 137.2, 133.0, 132.4, 128.0, 127.5, 125.5, 123.6, 122.7, 119.7, 118.9, 118.7, 113.2, 110.2, 109.9, 46.4, 19.8; LRMS (FAB) m/z, 421 ([M+H]⁺).

### EV112

:Following the general conditions of the N-alkylation, a yellow solid EV112 (279.0 mg, 82% yield) was obtained using Evodiamine (1) (250.0 mg, 0.814 mmol) and cyclohexanecarbonyl chloride (241.6 mg, 1.648 mmol).

m.p.: 91-93° C.; ¹H-NMR (400 MHz, DMSO-d₆) δ 7.98 (d, J=8.6 Hz, 1H), 7.92 (dd, J=7.6, 1.5 Hz, 1H), 7.68 (d, J=7.9 Hz, 1H), 7.52-7.57 (m, 1H), 7.39-7.43 (m, 1H), 7.32 (t, J=7.3 Hz, 1H), 7.17-7.23 (m, 2H), 6.33 (s, 1H), 4.67 (dd, J=13.1, 4.0 Hz, 1H), 3.17-3.30 (m, 2H), 3.02-3.06 (m, 1H), 2.80 (tt, J=12.8, 3.1 Hz, 1H), 2.29 (s, 3H), 2.06 (d, J=12.8 Hz, 1H), 1.89 (d, J=12.2 Hz, 1H), 1.74-1.76 (m, 1H), 1.58-1.69 (m, 3H), 1.38-1.47 (m, 1H), 1.17-1.30 (m, 2H); ¹³C-NMR (100 MHz, DMSO-d₆) δ 176.5, 163.4, 150.1, 136.4, 133.1, 128.3, 128.1, 127.2, 125.7, 123.4, 123.0, 123.0, 121.9, 121.4, 119.3, 114.9, 68.2, 44.7, 37.5, 35.8, 30.5, 28.2, 25.3, 25.2, 24.6, 20.0; LRMS (FAB) m/z, 413 ([M+H]⁺).

### Synthesis Example 3. Synthesis of D-ring Evodiamine Derivatives

(EV201-222)

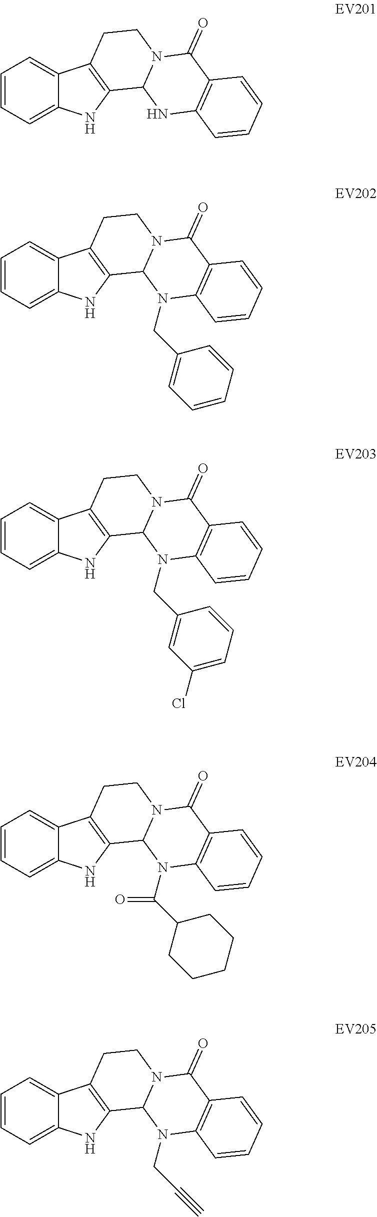 [SMILES: O=C1c2ccccc2NC2c3[nH]c4ccccc4c3CCN12 ; O=C1c2ccccc2N(Cc2ccccc2)C2c3[nH]c4ccccc4c3CCN12 ; O=C1c2ccccc2N(Cc2cccc(Cl)c2)C2c3[nH]c4ccccc4c3CCN12 ; O=C1c2ccccc2N(C(=O)C2CCCCC2)C2c3[nH]c4ccccc4c3CCN12 ; C#CCN1c2ccccc2C(=O)N2CCc3c([nH]c4ccccc34)C21]

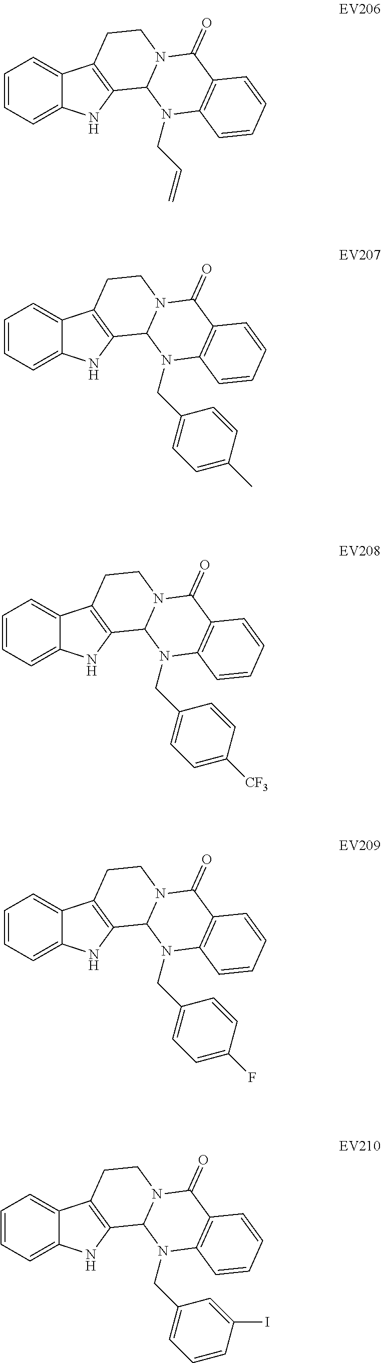 [SMILES: C=CCN1c2ccccc2C(=O)N2CCc3c([nH]c4ccccc34)C21 ; Cc1ccc(CN2c3ccccc3C(=O)N3CCc4c([nH]c5ccccc45)C32)cc1 ; O=C1c2ccccc2N(Cc2ccc(C(F)(F)F)cc2)C2c3[nH]c4ccccc4c3CCN12 ; O=C1c2ccccc2N(Cc2ccc(F)cc2)C2c3[nH]c4ccccc4c3CCN12 ; O=C1c2ccccc2N(Cc2cccc(I)c2)C2c3[nH]c4ccccc4c3CCN12]

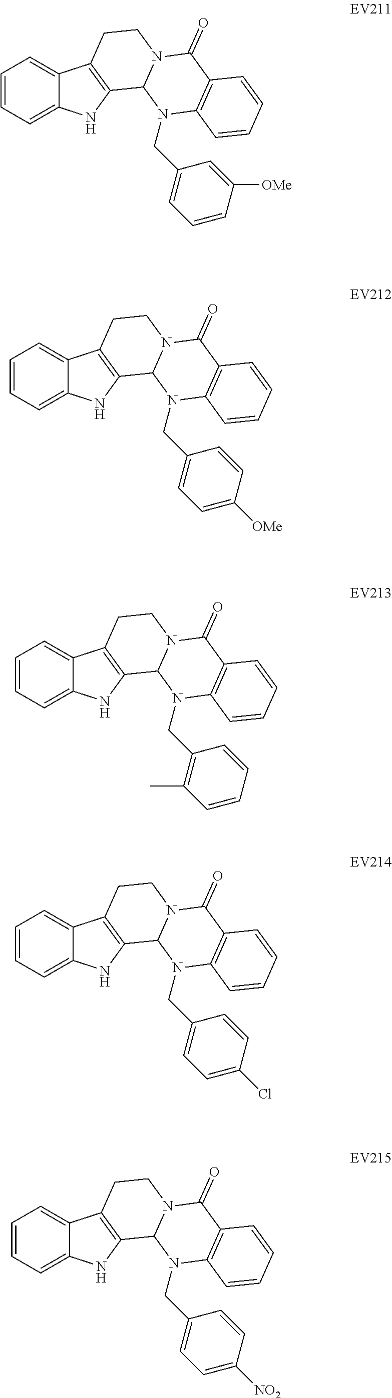 [SMILES: COc1cccc(CN2c3ccccc3C(=O)N3CCc4c([nH]c5ccccc45)C32)c1 ; COc1ccc(CN2c3ccccc3C(=O)N3CCc4c([nH]c5ccccc45)C32)cc1 ; Cc1ccccc1CN1c2ccccc2C(=O)N2CCc3c([nH]c4ccccc34)C21 ; O=C1c2ccccc2N(Cc2ccc(Cl)cc2)C2c3[nH]c4ccccc4c3CCN12 ; O=C1c2ccccc2N(Cc2ccc([N+](=O)[O-])cc2)C2c3[nH]c4ccccc4c3CCN12]

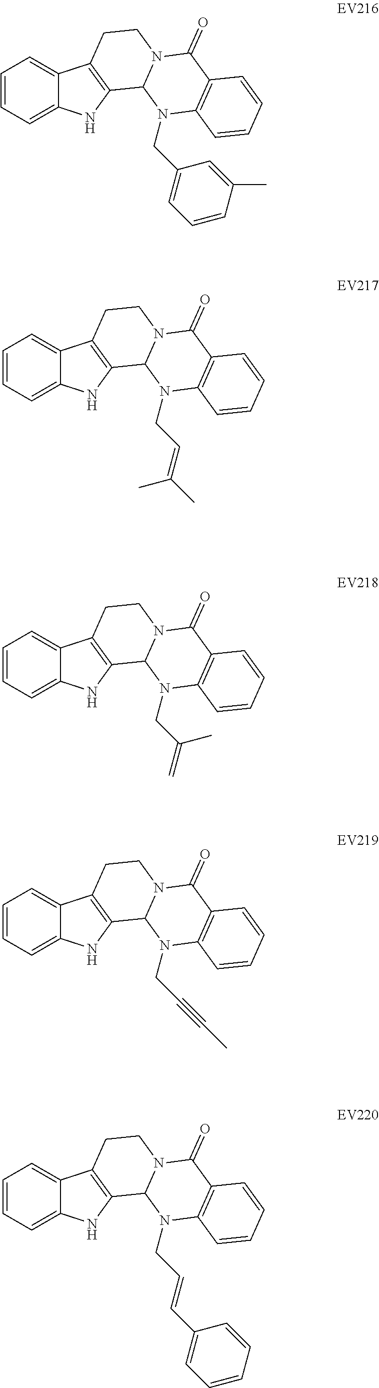 [SMILES: Cc1cccc(CN2c3ccccc3C(=O)N3CCc4c([nH]c5ccccc45)C32)c1 ; CC(C)=CCN1c2ccccc2C(=O)N2CCc3c([nH]c4ccccc34)C21 ; C=C(C)CN1c2ccccc2C(=O)N2CCc3c([nH]c4ccccc34)C21 ; CC#CCN1c2ccccc2C(=O)N2CCc3c([nH]c4ccccc34)C21 ; O=C1c2ccccc2N(C/C=C/c2ccccc2)C2c3[nH]c4ccccc4c3CCN12]

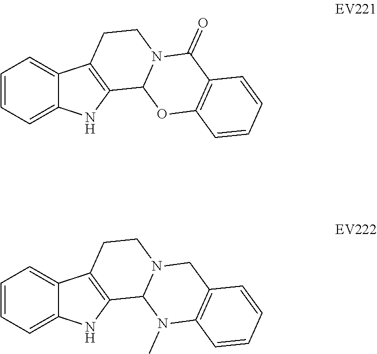 [SMILES: O=C1c2ccccc2OC2c3[nH]c4ccccc4c3CCN12 ; CN1c2ccccc2CN2CCc3c([nH]c4ccccc34)C21]

### Method for Synthesis of EV201˜220 Derivatives

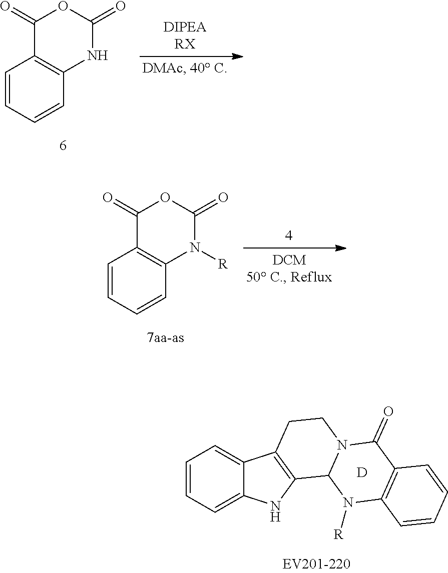

### N-Propargylisatoic Anhydride (7Ae)

:Using isatoic anhydride (6) (200.0 mg, 1.226 mmol) and propargyl bromide (437.5 mg, 3.678 mmol) under the synthesis conditions for the N-methylisatoic anhydride (7), a beige solid compound 7ae (166.6 mg, 68% yield) was obtained.

m.p.: 176-177° C.; ¹H-NMR (400 MHz, DMSO-d₆) δ 8.04 (d, J=7.8 Hz, 1n), 7.91 (t, J=7.8 Hz, 1H), 7.51 (d, J=8.7 Hz, 1H), 7.38 (t, J=7.6 Hz, 1H), 4.89 (s, 2H), 3.43 (s, 1H); ¹³C-NMR (100 MHz, DMSO-d₆) δ 158.5, 147.3, 140.5, 137.2, 129.6, 124.1, 115.0, 111.9, 77.6, 76.1, 34.4; LRMS (FAB) m/z, 202 ([M+H]⁺).

### N-allylisatoic Anhydride (7Af)

:Using isatoic anhydride (6) (200.0 mg, 1.226 mmol) and allyl bromide (445.0 mg, 3.678 mmol) under the synthesis conditions for the N-methylisatoic anhydride (7), a beige solid compound 7af (106.0 mg, 50% yield) was obtained.

m.p.: 127-128° C.; ¹H-NMR (400 MHz, DMSO-d₆) δ 8.02 (dd, J=8.0, 1.1 Hz, 1H), 7.79-7.84 (m, 1H), 7.31-7.35 (m, 2H), 5.87-5.96 (m, 1H), 5.31 (dq, J=17.5, 1.7 Hz, 1H), 5.20 (dq, J=10.6, 1.5 Hz, 1H), 4.67 (td, J=3.2, 1.5 Hz, 2H); ¹³C-NMR (100 MHz, DMSO-d₆) δ 159.0, 147.6, 141.3, 137.0, 131.2, 129.5, 123.6, 117.2, 115.2, 111.9, 46.5, 40.2, 39.9, 39.7, 39.5, 39.3, 39.1, 38.90; LRMS (FAB) m/z, 334 ([M+H]⁺).

### N-(2-methyl)allylisatoic anhydride (7aq)

:Using isatoic anhydride (6) (200.0 mg, 1.226 mmol) and 3-bromo-2-methylpropene (496.6 mg, 3.678 mmol) under the synthesis conditions for the N-methylisatoic anhydride (7), a beige solid compound 7aq (151.6 mg, 57% yield) was obtained.

m.p.: 124-125° C.; ¹H-NMR (400 MHz, DMSO-d₆) δ 8.02 (dd, J=7.7, 1.5 Hz, 1H), 7.78-7.82 (m, 1H), 7.31-7.35 (m, 1H), 7.24 (d, J=8.6 Hz, 1H), 4.82-4.87 (m, 2H), 4.55 (s, 2H), 1.80 (s, 3H); ¹³C-NMR (100 MHz, DMSO-d₆) δ 159.0, 147.8, 141.5, 138.2, 136.9, 129.4, 123.7, 115.4, 111.8, 111.0, 49.5, 19.8; LRMS (FAB) m/z, 334 ([M+H]⁺).

### EV201

:Using 3,4-dihydro-β-carboline (4) (200 mg, 1.175 mmol) and isatoic anhydride (6) (174.3 mg, 1.068 mmol) under the synthesis conditions for the Evodiamine (1), a white solid compound EV201 (153.0 mg, 50% yield) was obtained.

m.p.: 210-212° C.; ¹H-NMR (400 MHz, DMSO-d₆) δ 10.91 (s, 1H), 7.76 (dd, J=7.8, 1.4 Hz, 1H), 7.51 (d, J=7.8 Hz, 1H), 7.43 (d, J=8.3 Hz, 1H), 7.33-7.38 (m, 1H), 7.14 (td, J=7.5, 1.1 Hz, 1H), 7.04 (td, J=7.4, 1.0 Hz, 1H), 6.90 (s, 1H), 6.82-6.88 (m, 2H), 6.05 (d, J=1.4 Hz, 1H), 4.80 (dd, J=12.9, 3.7 Hz, 1H), 3.02 (td, J=12.2, 4.7 Hz, 1H), 2.73-2.86 (m, 2H); ¹³C-NMR (100 MHz, DMSO-d₆) δ 163.7, 147.3, 136.3, 133.3, 130.8, 128.2, 125.9, 121.9, 119.0, 118.6, 118.5, 116.1, 115.4, 115.3, 111.7, 109.3, 63.8, 38.7, 20.1; LRMS (FAB) m/z, 290 ([M+H]⁺).

### EV202

:Using 3,4-dihydro-β-carboline (4) (74.0 mg, 0.434 mmol) and N-benzylisatoic anhydride (7aa) (100.0 mg, 0.395 mmol) under the synthesis conditions for the Evodiamine (1), a yellow solid compound EV202 (29.6 mg, 20% yield) was obtained.

m.p.: 251-252° C.; ¹H-NMR (400 MHz, DMSO-d₆) δ 11.16 (s, 1H), 7.73-7.75 (m, 1H), 7.44 (d, J=7.8 Hz, 1H), 7.28-7.37 (m, 6H), 7.15-7.25 (m, 1H), 7.09 (t, J=7.3 Hz, 1H), 6.99 (t, J=7.5 Hz, 1H), 6.84 (t, J=8.0 Hz, 2H), 6.34 (s, 1H), 4.57-4.70 (m, 3H), 3.25 (td, J=12.3, 4.8 Hz, 1H), 2.88-2.96 (m, 1H), 2.71 (dd, J=15.6, 4.6 Hz, 1H); ¹³C-NMR (100 MHz, DMSO-d₆) δ 164.8, 146.7, 138.3, 136.3, 133.2, 131.6, 128.5, 128.3, 127.6, 127.2, 126.3, 122.0, 119.8, 119.0, 118.3, 117.3, 111.7, 111.4, 70.4, 52.9, 41.8, 19.3; LRMS (FAB) m/z, 380 ([M+H]⁺).

### EV203

:Using 3,4-dihydro-β-carboline (4) (39.0 mg, 0.229 mmol) and N-(3-chloro)benzylisatoic anhydride (7ab) (60.0 mg, 0.209 mmol) under the synthesis conditions for the Evodiamine (1), a pale yellow solid compound EV203 (26.2 mg, 30% yield) was obtained.

m.p.: 230-232° C.; ¹H-NMR (400 MHz, DMSO-d₆) δ 11.21 (s, 1n), 7.78 (d, J=7.8 Hz, 1H), 7.46 (d, J=7.8 Hz, 1H), 7.21-7.37 (m, 6H), 7.11 (t, J=7.5 Hz, 1H), 7.00 (t, J=7.3 Hz, 1H), 6.90 (t, J=8.2 Hz, 2H), 6.34 (s, 1H), 4.54-4.64 (m, 3H), 3.25 (td, J=12.1, 4.4 Hz, 1H), 2.87-2.95 (m, 1H), 2.75 (dd, J=15.3, 3.9 Hz, 1H); ¹³C-NMR (100 MHz, DMSO-d₆) δ 164.7, 146.7, 141.0, 136.3, 133.3, 133.1, 131.1, 130.2, 128.3, 127.5, 127.2, 126.3, 126.2, 121.9, 120.4, 119.5, 119.0, 118.2, 117.6, 111.7, 111.6, 70.1, 52.3, 41.6, 19.3. LRMS (FAB) m/z, 414 ([M+H]⁺).

### EV204

:Using 3,4-dihydro-β-carboline (4) (711.4 mg, 4.180 mmol) and N-cyclohexanecarbonylisatoic anhydride (7ac) (1.0 g, 3.800 mmol) under the synthesis conditions for the Evodiamine (1), a pale yellow solid compound EV204 (237.1 mg, 14% yield) was obtained.

m.p.: 243-244° C.; ¹H-NMR (400 MHz, DMSO-d₆) δ 11.16 (s, 1H), 7.82 (d, J=7.8 Hz, 1H), 7.58 (t, J=7.1 Hz, 2H), 7.34 (d, J=7.8 Hz, 1H), 7.31 (s, 1H), 7.24 (t, J=3.9 Hz, 2H), 7.01-7.05 (m, 1H), 6.92-6.95 (m, 1H), 4.65 (dd, J=13.0, 5.9 Hz, 1H), 3.64 (s, 1H), 2.91-3.04 (m, 2H), 2.58-2.67 (m, 1H), 2.08 (s, 1H), 1.79 (d, J=11.9 Hz, 1H), 1.63 (d, J=9.2 Hz, 4H), 1.37 (s, 2H), 1.21 (q, J=12.4 Hz, 2H); ¹³C-NMR (100 MHz, DMSO-d₆) δ 160.7, 147.4, 137.6, 135.7, 126.6, 122.2, 121.7, 118.9, 118.1, 111.5, 110.8, 117.0, 115.2, 111.7, 110.9, 71.0, 55.5, 42.5, 36.2, 29.7, 29.2, 25.4, 25.2, 24.8, 19.2; LRMS (FAB) m/z, 400 ([M+H]⁺).

### EV205

:Using 3,4-dihydro-β-carboline (4) (93.1 mg, 0.547 mmol) and N-propargylisatoic anhydride (7ad) (100.0 mg, 0.497 mmol) under the synthesis conditions for the Evodiamine (1), a yellow solid compound EV205 (159.2 mg, 98% yield) was obtained.

m.p.: 182-184° C.; ¹H-NMR (400 MHz, DMSO-d₆) δ 11.26 (s, 1H), 7.90 (dd, J=7.8, 1.4 Hz, 1H), 7.50-7.56 (m, 2H), 7.38 (d, J=8.3 Hz, 1H), 7.31 (d, J=7.4 Hz, 1H), 7.12-7.19 (m, 2H), 7.02-7.06 (m, 1H), 6.16 (s, 1H), 4.62 (ddd, J=12.8, 4.9, 2.0 Hz, 1H), 4.04 (dd, J=18.2, 2.5 Hz, 1H), 3.61 (dd, J=17.9, 2.3 Hz, 1H), 3.18-3.25 (m, 1H), 3.04 (t, J=2.3 Hz, 1H), 2.84-2.98 (m, 2H); ¹³C-NMR (100 MHz, DMSO-d₆) δ 163.5, 147.1, 136.8, 132.8, 129.0, 128.0, 125.8, 123.4, 122.9, 122.1, 121.3, 119.0, 118.5, 112.2, 111.7, 79.9, 75.2, 68.1, 19.5; HRMS (FAB) m/z calcd for C₂₁H₁₈N₃O [M+H]⁺: 328.1450, found 328.1456.

### EV206

:Using 3,4-dihydro-β-carboline (4) (92.2 mg, 0.541 mmol) and N-allylisatoic anhydride (7ae) (100.0 mg, 0.492 mmol) under the synthesis conditions for the Evodiamine (1), a yellow solid compound EV206 (125.1 mg, 84% yield) was obtained.

m.p.: 204-206° C.; ¹H-NMR (400 MHz, DMSO-d₆) δ 11.08 (s, 1H), 7.79 (d, J=7.8 Hz, 1H), 7.42-7.47 (m, 2H), 7.35 (d, J=7.8 Hz, 1H), 7.11 (dd, J=7.6, 5.3 Hz, 2H), 6.97 (td, J=15.1, 7.8 Hz, 2H), 6.15 (s, 1H), 5.85 (dq, J=22.4, 5.4 Hz, 1H), 5.02-5.07 (m, 2H), 4.62 (dd, J=12.9, 5.1 Hz, 1H), 3.94 (d, 2H), 3.23 (td, J=12.2, 4.6 Hz, 1H), 2.90-2.98 (m, 1H), 2.76 (dd, J=15.6, 4.1 Hz, 1H). ¹³C-NMR (100 MHz, DMSO-d₆) b 164.5, 147.2, 136.4, 134.5, 133.1, 130.8, 128.0, 126.1, 121.8, 120.4, 120.0, 118.9, 118.5, 118.2, 117.1, 111.7, 111.4, 69.0, 52.6, 41.2, 19.3; HRMS (FAB) m/z calcd for C₂₁H₂ON₃O [M+H]⁺: 330.1606, found 330.1607.

### EV207

Using 3,4-dihydro-β-carboline (4) (35.1 mg, 0.206 mmol) and N-(4-methyl)benzylisatoic anhydride (7af) (50.0 mg, 0.187 mmol) under the synthesis conditions for the Evodiamine (1), a yellow solid compound EV207 (45.1 mg, 61% yield) was obtained.

m.p.: 250-252° C.; ¹H-NMR (400 MHz, DMSO-d₆) δ 11.12 (s, 1H), 7.74 (dd, J=8.0, 1.6 Hz, 1H), 7.44 (d, J=7.8 Hz, 1H), 7.29-7.37 (m, 2H), 7.17 (d, J=7.8 Hz, 2H), 7.08-7.12 (m, 3H), 6.97-7.01 (m, 1H), 6.83-6.87 (m, 2H), 6.31 (s, 1H), 4.51-4.63 (m, 3H), 3.24 (td, J=12.2, 4.6 Hz, 1H), 2.88-2.96 (m, 1H), 2.72 (dd, J=15.4, 4.8 Hz, 1H), 2.24 (s, 3H); ¹³C-NMR (100 MHz, DMSO-d₆) δ 164.8, 146.8, 136.3, 136.3, 135.1, 133.2, 131.6, 129.0, 128.1, 127.6, 126.3, 121.8, 119.8, 119.0, 118.9, 118.2, 117.4, 111.7, 111.4, 70.3, 52.6, 41.8, 20.7, 19.3. LRMS (FAB) m/z, 394 ([M+H]⁺).

### EV208

:Using 3,4-dihydro-β-carboline (4) (29.2 mg, 0.171 mmol) and N-(4-trifluoromethyl)benzylisatoic anhydride (7ag) (50.0 mg, 0.156 mmol) under the synthesis conditions for the Evodiamine (1), a yellow solid compound EV208 (47.1 mg, 68% yield) was obtained.

m.p.: 207-209° C.; ¹H-NMR (400 MHz, DMSO-d₆) δ 11.14 (s, 1H), 7.78 (dd, J=7.8, 1.4 Hz, 1H), 7.64 (d, J=7.8 Hz, 2H), 7.47 (dd, J=21.1, 7.8 Hz, 3H), 7.33-7.37 (m, 2H), 7.08-7.12 (m, 1H), 6.97-7.01 (m, 1H), 6.86-6.91 (m, 2H), 6.36 (s, 1H), 4.60-4.78 (m, 3H), 3.22-3.30 (m, 1H), 2.89-2.97 (m, 1H), 2.74 (dd, J=15.6, 4.6 Hz, 1H); ¹³C-NMR (100 MHz, DMSO-d₆) δ 164.7, 146.7, 143.3, 136.3, 133.4, 131.2, 128.3, 128.2, 126.2, 125.3, 125.2, 121.9, 120.2, 119.2, 119.0, 118.2, 117.2, 111.7, 111.6, 70.2, 52.3, 41.7, 19.3; LRMS (FAB) m/z, 448 ([M+H]⁺).

### EV209

:Using 3,4-dihydro-β-carboline (4) (34.5 mg, 0.203 mmol) and N-(4-fluoromethyl)benzylisatoic anhydride (7ah) (50.0 mg, 0.184 mmol) under the synthesis conditions for the Evodiamine (1), a yellow solid compound EV209 (47.7 mg, 65% yield) was obtained.

m.p.: 224-226° C.; ¹H-NMR (400 MHz, DMSO-d₆) δ 11.16 (s, 1H), 7.77 (dd, J=7.8, 1.4 Hz, 1H), 7.45 (d, J=7.8 Hz, 1H), 7.27-7.37 (m, 4H), 7.08-7.13 (m, 2H), 6.98-7.02 (m, 1H), 6.85-6.91 (m, 2H), 6.32 (s, 1H), 4.50-4.62 (m, 3H), 3.21-3.30 (m, 1H), 2.87-2.95 (m, 1H), 2.74 (dd, J=15.4, 4.8 Hz, 1H); ¹³C-NMR (100 MHz, DMSO-d₆) δ 164.7, 146.8, 136.3, 134.4, 134.4, 133.2, 131.3, 129.6, 129.6, 128.2, 126.2, 121.9, 120.3, 119.5, 119.0, 118.2, 117.7, 115.3, 115.1, 111.7, 111.5, 70.1, 52.1, 41.6, 19.3; LRMS (FAB) m/z, 398 ([M+H]⁺).

### EV210

:Using 3,4-dihydro-β-carboline (4) (24.7 mg, 0.145 mmol) and N-(3-iodo)benzylisatoic anhydride (7ai) (50.0 mg, 0.132 mmol) under the synthesis conditions for the Evodiamine (1), a pale yellow solid compound EV210 (34.8 mg, 52% yield) was obtained.

m.p.: 255-257° C.; ¹H-NMR (400 MHz, DMSO-d₆) δ 11.19 (s, 1H), 7.78 (dd, J=8.0, 1.6 Hz, 1H), 7.57 (d, J=7.4 Hz, 2H), 7.46 (d, J=7.8 Hz, 1H), 7.36 (td, J=7.7, 2.0 Hz, 2H), 7.25 (d, J=7.8 Hz, 1H), 6.98-7.13 (m, 3H), 6.89-6.93 (m, 2H), 6.31 (s, 1H), 4.54-4.62 (m, 3H), 3.23 (td, J=12.2, 4.3 Hz, 1H), 2.85-2.93 (m, 1H), 2.75 (dd, J=15.4, 4.8 Hz, 1H); ¹³C-NMR (100 MHz, DMSO-d₆) δ 164.6, 146.8, 141.0, 136.3, 136.3, 135.9, 133.3, 131.0, 130.5, 128.2, 127.0, 126.2, 121.9, 120.4, 119.6, 119.0, 118.3, 117.7, 111.7, 111.6, 95.0, 70.0, 52.1, 41.5, 19.3; LRMS (FAB) m/z, 506 ([M+H]⁺).

### EV211

:Using 3,4-dihydro-β-carboline (4) (33.1 mg, 0.195 mmol) and N-(3-methoxy)benzylisatoic anhydride (7aj) (50.0 mg, 0.177 mmol) under the synthesis conditions for the Evodiamine (1), a white solid compound EV211 (66.7 mg, 92% yield) was obtained.

m.p.: 212-214° C.; ¹H-NMR (400 MHz, DMSO-d₆) δ 11.16 (s, 1H), 7.76 (dd, J=7.5, 1.4 Hz, 1H), 7.45 (d, J=7.4 Hz, 1H), 7.31-7.37 (m, 2H), 7.20 (t, J=7.7 Hz, 1H), 7.08-7.12 (m, 1H), 6.98-7.02 (m, 1H), 6.77-6.90 (m, 5H), 6.32 (s, 1H), 4.58-4.64 (m, 3H), 3.66 (s, 3H), 3.21-3.28 (m, 1H), 2.86-2.94 (m, 1H), 2.73 (dd, J=15.6, 4.6 Hz, 1H); ¹³C-NMR (100 MHz, DMSO-d₆) δ 164.7, 159.3, 146.9, 139.9, 136.3, 133.2, 131.3, 129.5, 128.1, 126.2, 121.8, 119.9, 119.7, 119.1, 118.9, 118.2, 117.3, 113.2, 112.4, 111.7, 111.4, 70.1, 54.9, 52.7, 41.6, 19.3; LRMS (FAB) m/z, 410 ([M+H]⁺).

### EV212

:Using 3,4-dihydro-β-carboline (4) (33.1 mg, 0.195 mmol) and N-(4-methoxy)benzylisatoic anhydride (7ak) (50.0 mg, 0.177 mmol) under the synthesis conditions for the Evodiamine (1), a white solid compound EV212 (24.1 mg, 33% yield) was obtained.

m.p.: 245-247° C.; ¹H-NMR (400 MHz, DMSO-d₆) δ 11.13 (s, 1H), 7.75 (dd, J=8.3, 1.5 Hz, 1H), 7.45 (d, J=8.0 Hz, 1H), 7.30-7.37 (m, 2H), 7.19 (d, J=8.6 Hz, 2H), 7.10 (t, J=7.1 Hz, 1H), 7.00 (t, J=7.4 Hz, 1H), 6.83-6.88 (m, 4H), 6.30 (s, 1H), 4.46-4.62 (m, 3H), 3.70 (s, 3H), 3.24 (td, J=12.3, 4.5 Hz, 1H), 2.89-2.97 (m, 1H), 2.72 (dd, J=15.3, 4.3 Hz, 1H); ¹³C-NMR (100 MHz, DMSO-d₆) δ 164.7, 158.4, 146.9, 137.0, 136.3, 133.1, 131.5, 129.9, 129.0, 128.1, 126.2, 121.8, 120.0, 119.3, 118.9, 118.2, 117.7, 114.1, 113.8, 111.7, 111.4, 70.1, 55.0, 52.3, 41.7, 19.3. LRMS (FAB) m/z, 410 ([M+H]⁺).

### EV213

:Using 3,4-dihydro-β-carboline (4) (35.1 mg, 0.206 mmol) and N-(2-methyl)benzylisatoic anhydride (7al) (50.0 mg, 0.187 mmol), under the synthesis conditions for the Evodiamine (1), a pale yellow solid compound, EV213 (49.7 mg, 68% yield) was obtained.

m.p.: 279-281° C.; ¹H-NMR (400 MHz, DMSO-d₆) δ 11.06 (s, 1H), 7.74 (dd, J=7.7, 1.5 Hz, 1H), 7.42 (d, J=8.0 Hz, 1H), 7.36 (d, J=8.0 Hz, 1H), 7.25-7.31 (m, 2H), 7.08-7.19 (m, 4H), 6.99 (t, J=7.4 Hz, 1H), 6.79 (t, J=7.7 Hz, 1H), 6.66 (d, J=8.6 Hz, 1H), 6.29 (s, 1H), 4.92 (d, J=16.6 Hz, 1H), 4.65 (q, J=6.3 Hz, 1H), 4.52 (d, J=15.9 Hz, 1H), 3.26 (td, J=12.3, 4.7 Hz, 1H), 2.96-3.04 (m, 1H), 2.68 (dd, J=15.3, 4.9 Hz, 1H), 2.30 (s, 3H); 13C-NMR (100 MHz, DMSO-d₆) δ 165.0, 146.4, 136.1, 136.0, 135.6, 133.4, 132.3, 130.3, 128.1, 127.1, 127.1, 126.4, 125.9, 121.8, 119.1, 119.0, 118.1, 117.8, 115.9, 111.6, 111.3, 70.5, 50.1, 42.4, 19.1, 18.9; LRMS (FAB) m/z, 394 ([M+H]⁺).

### EV214

Using 3,4-dihydro-β-carboline (4) (32.6 mg, 0.191 mmol) and N-(4-chloro)benzylisatoic anhydride (7am) (50.0 mg, 0.174 mmol), under the synthesis conditions for the Evodiamine (1), a pale yellow solid compound, EV214 (48.3 mg, 67% yield) was obtained.

m.p.: 265-267° C.; ¹H-NMR (400 MHz, DMSO-d₆) δ 11.15 (s, 1H), 7.77 (dd, J=8.0, 1.2 Hz, 1H), 7.45 (d, J=8.0 Hz, 1H), 7.27-7.36 (m, 6H), 7.08-7.12 (m, 1H), 6.98-7.02 (m, 1H), 6.85-6.91 (m, 2H), 6.33 (s, 1H), 4.52-4.63 (m, 3H), 3.24 (td, J=12.3, 4.5 Hz, 1H), 2.87-2.95 (m, 1H), 2.74 (dd, J=15.3, 4.9 Hz, 1H); ¹³C-NMR (100 MHz, DMSO-d₆) δ 164.6, 146.7, 137.3, 136.3, 133.2, 131.7, 131.2, 129.5, 128.3, 128.2, 126.2, 121.9, 120.3, 119.4, 118.9, 118.2, 117.6, 111.7, 111.5, 70.1, 52.2, 41.6, 19.3; LRMS (FAB) m/z, 414 ([M+H]⁺).

### EV215

:Using 3,4-dihydro-β-carboline (4) (31.4 mg, 0.184 mmol) and N-(4-nitro)benzylisatoic anhydride (7an) (50.0 mg, 0.168 mmol), under the synthesis conditions for the Evodiamine (1), a greenish-yellow solid compound, EV215 (13.7 mg, 19% yield) was obtained.

m.p.: 228-230° C.; ¹H-NMR (400 MHz, DMSO-d₆) δ 11.14 (s, 1H), 8.12 (dd, J=9.2, 2.5 Hz, 2H), 7.80 (dd, J=7.7, 1.5 Hz, 1H), 7.54 (d, J=8.6 Hz, 2H), 7.45 (d, J=8.0 Hz, 1H), 7.31-7.38 (m, 2H), 7.07-7.10 (m, 1H), 6.89-7.01 (m, 3H), 6.36 (s, 1H), 4.61-4.76 (m, 3H), 3.24 (td, J=12.3, 4.5 Hz, 1H), 2.89-2.97 (m, 1H), 2.76 (dd, J=15.3, 4.9 Hz, 1H); ¹³C-NMR (100 MHz, DMSO-d₆) δ 164.6, 146.9, 146.6, 146.5, 136.3, 133.4, 130.8, 128.6, 128.3, 127.9, 126.1, 123.7, 123.4, 121.9, 120.7, 119.7, 119.0, 118.3, 117.6, 111.7, 111.6, 70.0, 52.3, 41.5, 19.3; LRMS (FAB) m/z, 425 ([M+H]⁺).

### EV216

:Using 3,4-dihydro-β-carboline (4) (35.1 mg, 0.206 mmol) and N-(3-methyl)benzylisatoic anhydride (7ao) (50.0 mg, 0.187 mmol), under the synthesis conditions for the Evodiamine (1), a white solid compound, EV216 (45 mg, 61% yield) was obtained.

m.p.: 243-245° C.; ¹H-NMR (400 MHz, DMSO-d₆) δ 11.12 (s, 1H), 7.74-7.77 (m, 1H), 7.45 (d, J=7.4 Hz, 1H), 7.30-7.37 (m, 2H), 7.17 (t, J=7.4 Hz, 1H), 6.98-7.12 (m, 5H), 6.86 (dd, J=7.7, 6.4 Hz, 2H), 6.31 (s, 1H), 4.53-4.63 (m, 3H), 3.25 (td, J=12.3, 4.5 Hz, 1H), 2.87-2.95 (m, 1H), 2.72 (dd, J=15.3, 4.9 Hz, 1H), 2.23 (s, 3H); ¹³C-NMR (100 MHz, DMSO-d₆) δ 164.7, 146.9, 138.1, 137.4, 136.3, 133.2, 131.4, 128.3, 128.2, 128.1, 127.8, 126.2, 124.6, 121.8, 119.8, 119.0, 118.9, 118.2, 117.2, 111.7, 111.5, 70.2, 52.8, 41.7, 21.0, 19.2; LRMS (FAB) m/z, 394 ([M+H]⁺).

### EV217

:Using 3,4-dihydro-β-carboline (4) (40.5 mg, 0.238 mmol) and N-(1-dimethyl)allylisatoic anhydride (7ap) (50.0 mg, 0.216 mmol), under the synthesis conditions for the Evodiamine (1), a white solid compound, EV217 (67 mg, 87% yield) was obtained.

m.p.: 219-221° C.; ¹H-NMR (400 MHz, DMSO-d₆) δ 11.10 (s, 1H), 7.79 (dd, J=8.0, 1.2 Hz, 1H), 7.43-7.47 (m, 2H), 7.35 (d, J=8.0 Hz, 1H), 7.10 (t, J=7.7 Hz, 2H), 6.95-7.02 (m, 2H), 6.11 (s, 1H), 5.17 (t, J=6.7 Hz, 1H), 4.58-4.63 (m, 1H), 3.86 (d, J=7.4 Hz, 2H), 3.19-3.26 (m, 1H), 2.75-2.91 (m, 2H), 1.57 (s, 3H), 1.33 (s, 3H); ¹³C-NMR (100 MHz, DMSO-d₆) δ 164.4, 147.7, 136.4, 134.2, 133.0, 130.7, 128.0, 126.2, 121.8, 120.8, 120.7, 120.7, 119.0, 118.9, 118.1, 111.6, 111.4, 68.8, 47.8, 40.9, 40.2, 39.9, 39.7, 39.5, 39.3, 39.1, 38.9, 25.5, 19.4, 17.50; LRMS (FAB) m/z, 358 ([M+H]⁺).

### EV218

:Using 3,4-dihydro-β-carboline (4) (43.1 mg, 0.253 mmol) and N-(2-methyl)allylisatoic anhydride (7aq) (50.0 mg, 0.230 mmol), under the synthesis conditions for the Evodiamine (1), a dark yellow solid compound, EV218 (75.0 mg, 95% yield) was obtained.

m.p.: 181-183° C.; ¹H-NMR (400 MHz, DMSO-d₆) δ 10.94 (s, 1H), 7.72 (dd, J=7.9, 1.8 Hz, 1H), 7.32-7.43 (m, 3H), 7.08 (t, J=7.9 Hz, 1H), 6.98 (t, J=7.9 Hz, 1H), 6.91 (d, J=7.9 Hz, 1H), 6.80 (t, J=7.0 Hz, 1H), 6.20 (s, 1H), 4.88 (d, J=21.4 Hz, 2H), 4.62 (dd, J=13.4, 5.5 Hz, 1H), 4.22 (d, J=16.5 Hz, 1H), 3.94 (d, J=16.5 Hz, 1H), 3.26 (td, J=12.2, 4.9 Hz, 1H), 2.95-3.03 (m, 1H), 2.68 (dd, J=15.6, 4.6 Hz, 1H), 1.72 (s, 3H); ¹³C-NMR (100 MHz, DMSO-d₆) δ 164.9, 146.6, 141.3, 136.1, 133.3, 132.1, 128.0, 126.3, 121.7, 118.9, 118.8, 118.1, 117.4, 115.8, 112.3, 111.7, 111.2, 70.1, 54.9, 42.2, 20.0, 19.2; HRMS (FAB) m/z calcd for C₂₂H₂₂N₃O [M+H]⁺: 344.1763, found 344.1759.

### EV219

:Using 3,4-dihydro-β-carboline (4) (43.5 mg, 0.256 mmol) and N-(1-methyl)propargylisatoic anhydride (7ar) (50.0 mg, 0.232 mmol), under the synthesis conditions for the Evodiamine (1), a yellow solid compound, EV219 (74.2 mg, 94% yield) was obtained.

m.p.: 195-198° C.; ¹H-NMR (400 MHz, DMSO-d₆) δ 11.20 (s, 1H), 7.88 (dd, J=7.9, 1.2 Hz, 1H), 7.51-7.55 (m, 2H), 7.37 (d, J=7.9 Hz, 1H), 7.26 (d, J=7.9 Hz, 1H), 7.11-7.15 (m, 2H), 7.01-7.05 (m, 1H), 6.17 (s, 1H), 4.61-4.65 (m, 1H), 3.97 (dd, J=17.7, 2.4 Hz, 1H), 3.68 (dd, J=17.7, 2.4 Hz, 1H), 3.17-3.28 (m, 1H), 2.83-2.98 (m, 2H), 1.65 (t, J=2.1 Hz, 3H); ¹³C-NMR (100 MHz, DMSO-d₆) δ 163.6, 147.2, 136.7, 132.8, 129.4, 127.9, 125.9, 122.9, 122.5, 122.0, 120.8, 118.9, 118.4, 112.0, 111.7, 80.4, 75.2, 68.3, 40.2, 19.5, 3.1; LRMS (FAB) m/z, 342 ([M+H]⁺).

### EV220

:Using 3,4-dihydro-β-carboline (4) (33.6 mg, 0.197 mmol) and N-cinnamylisatoic anhydride (7as) (50.0 mg, 0.179 mmol), under the synthesis conditions for the Evodiamine (1), a pale yellow solid compound, EV220 (13.7 mg, 19% yield) was obtained.

m.p.: 194-196° C.; ¹H-NMR (400 MHz, DMSO-d₆) δ 11.18 (s, 1H), 7.80 (dd, J=7.9, 1.2 Hz, 1H), 7.47 (d, J=7.9 Hz, 1H), 7.35-7.44 (m, 2H), 7.25-7.33 (m, 5H), 7.17-7.20 (m, 1H), 7.11 (t, J=7.6 Hz, 1H), 6.95-7.03 (m, 2H), 6.24-6.43 (m, 2H), 6.21 (s, 1H), 4.62 (dd, J=13.1, 4.6 Hz, 1H), 4.07 (ddd, J=29.8, 15.7, 5.7 Hz, 2H), 3.25 (td, J=12.2, 4.5 Hz, 1H), 2.88-2.96 (m, 1H), 2.77 (dd, J=15.3, 4.3 Hz, 1H); ¹³C-NMR (100 MHz, DMSO-d₆) δ 164.5, 147.4, 136.4, 136.4, 133.2, 131.6, 130.7, 128.6, 128.0, 127.6, 126.3, 126.2, 126.1, 126.0, 121.9, 120.8, 120.5, 119.0, 118.9, 118.2, 111.7, 111.5, 68.9, 52.3, 41.1, 19.4; LRMS (FAB) m/z, 406 ([M+H]⁺).

### Method for Synthesis of EV221 Derivative

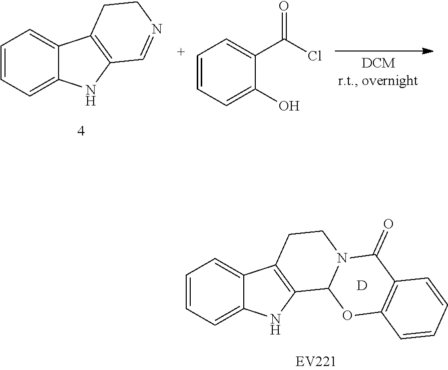

3,4-dihydro-β-carboline (2) (189.0 mg, 1.110 mmol) was dissolved in CH₂Cl₂ (3.0 mL, 0.4 M), followed by slow addition of salicyl chloride (226.7 mg, 1.448 mmol) under Ar substitution. After stirring at room temperature for 12 hours, the excess CH₂Cl₂ was removed by vacuum concentration. The resulting mixture was purified by column chromatography (silica gel, hexane:ethyl acetate=3:1˜2:1) to yield a white solid compound, EV221 (174.0 mg, 54% yield).

m.p.: 113-115° C.; ¹H-NMR (400 MHz, DMSO-d₆) δ 11.52 (s, 1H) 7.91 (d, J=7.6 Hz, 1H), 7.57-7.60 (m, 2H), 7.42 (d, J=8.2 Hz, 1H), 7.13-7.27 (m, 3H), 7.07 (t, J=7.7 Hz, 1H), 6.68 (s, 1H), 4.71-4.74 (m, 1H), 3.21-3.22 (m, 1H), 2.93-3.02 (m, 2H); ¹³C-NMR (100 MHz, DMSO-d₆) δ 162.6, 157.2, 137.4, 134.8, 128.5, 128.0, 125.7, 123.3, 123.1, 119.5, 119.3, 118.9, 117.0, 112.3, 112.1, 81.6, 20.3; LRMS (FAB) m/z, 291 ([M+H]⁺).

### Method for Synthesis of EV222 Derivative

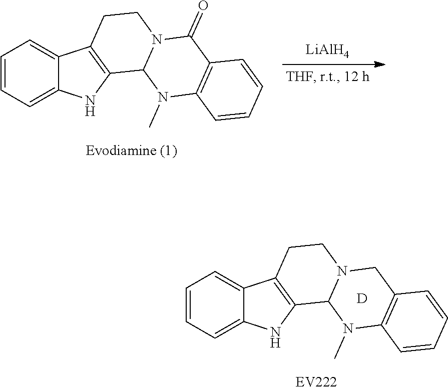

Evodiamine (1) (300.0 mg, 0.989 mmol) was dissolved in THF (30.0 mL), followed by addition of LiAlH₄ (110.0 mg, 2.967 mmol). After stirring at room temperature for 12 hours, water (0.2 mL) was added to terminate the reaction. The resulting mixture concentrated through vacuum was purified by column chromatography (silica gel, hexane:ethyl acetate=4:1-3:1) to yield a white solid compound, EV222 (182.4 mg, 64% yield).

m.p.: 162-164° C.; ¹H-NMR (400 MHz, DMSO-d₆) δ 11.22 (s, 1H), 7.45 (d, J=7.6 Hz, 1H), 7.35 (d, J=8.0 Hz, 1H), 7.06-7.15 (m, 2H), 6.93-7.02 (m, 3H), 6.83 (td, J=7.3, 1.0 Hz, 1H), 4.89 (s, 1H), 3.93 (s, 2H), 3.23-3.27 (m, 1H), 2.81-2.88 (m, 1H), 2.70-2.75 (m, 2H), 2.65 (s, 3H); ¹³C-NMR (100 MHz, DMSO-d₆) δ 147.5, 136.5, 131.5, 126.9, 126.7, 126.2, 124.9, 121.2, 119.9, 119.1, 118.5, 118.8, 111.4, 109.5, 72.6, 55.0, 48.6, 38.0, 20.8; LRMS (FAB) m/z, 291 ([M+H]⁺).

### Synthesis Example 4. Synthesis of E-Ring Evodiamine Derivatives (EV301˜312)

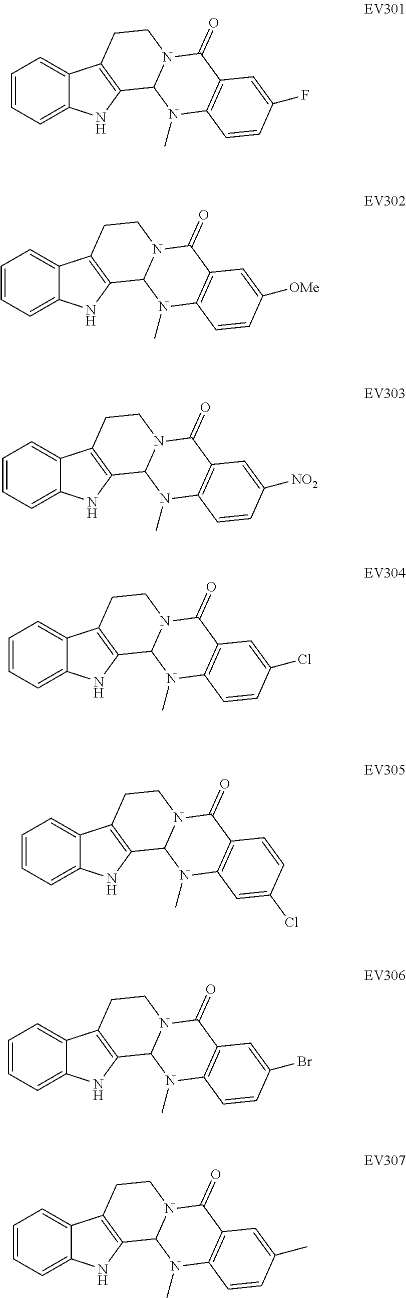

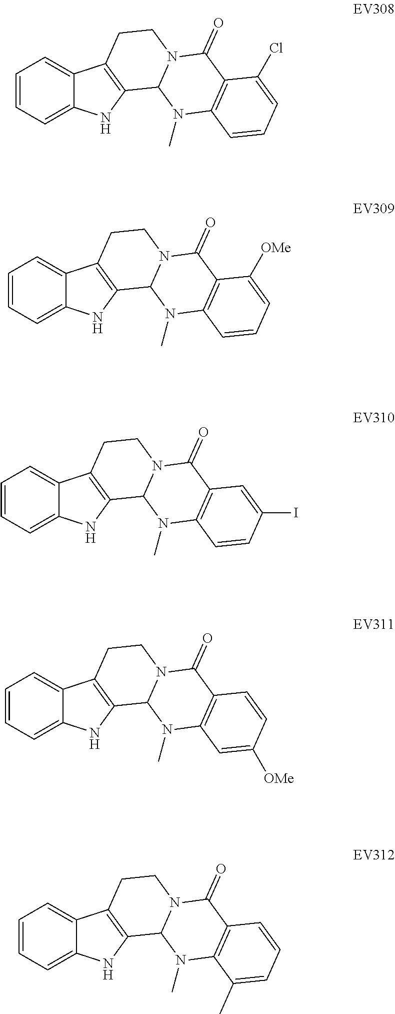 [SMILES: CN1c2cccc(Cl)c2C(=O)N2CCc3c([nH]c4ccccc34)C21 ; COc1cccc2c1C(=O)N1CCc3c([nH]c4ccccc34)C1N2C ; CN1c2ccc(I)cc2C(=O)N2CCc3c([nH]c4ccccc34)C21 ; COc1ccc2c(c1)N(C)C1c3[nH]c4ccccc4c3CCN1C2=O ; Cc1cccc2c1N(C)C1c3[nH]c4ccccc4c3CCN1C2=O]

### Method for Synthesis of EV301-312 Derivatives

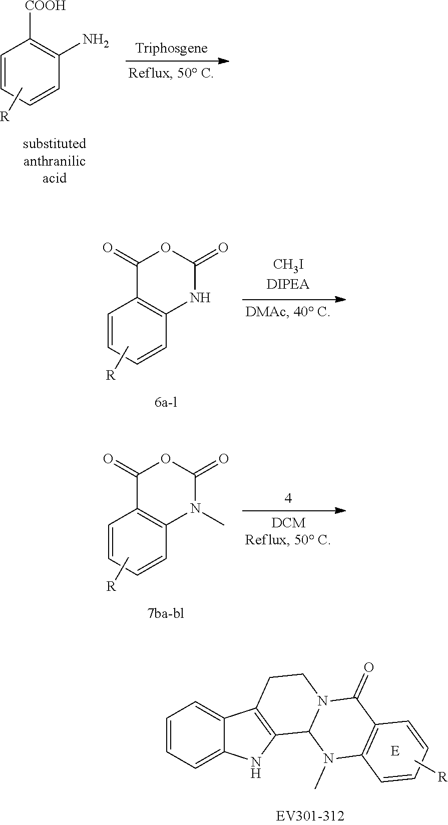

### 5-Fluoroisatoic Anhydride (6a)

:Using 5-fluoroanthranilic acid (300.0 mg, 1.932 mmol) under the synthesis conditions for the Isatoic anhydride (6), a white solid compound, 6a (350.0 mg, 99% yield) was obtained.

m.p.: 177-178° C.; ¹H-NMR (400 MHz, DMSO-d₆) δ 11.85 (d, J=17.5 Hz, 1H), 7.62-7.70 (m, 2H), 7.19-7.24 (m, 1H); ¹³C-NMR (100 MHz, DMSO-d₆) δ 159.3, 159.2, 158.8, 156.4, 146.9, 138.2, 124.9, 124.7, 117.6, 117.6, 114.2, 113.9, 111.6, 111.5; LRMS (FAB) m/z, 182 ([M+H]⁺).

### 5-Fluoro-N-methylisatoic anhydride (7ba)

:Using 6a (300.0 mg, 1.656 mmol) under the synthesis conditions for the N-methylisatoic anhydride (7), a beige solid compound, 7ba (193.9 mg, 60% yield), was obtained.

m.p.: 119-120° C.; ¹H-NMR (400 MHz, DMSO-d₆) δ 7.76 (td, J=7.8, 2.6 Hz, 2H), 7.49-7.52 (m, 1H), 3.46 (s, 3H); ¹³C-NMR (100 MHz, DMSO-d₆) δ 158.9, 158.3, 158.3, 156.5, 147.5, 139.1, 139.0, 124.7, 124.4, 117.5, 117.4, 114.7, 114.4, 113.0, 113.0, 32.0; LRMS (FAB) m/z, 196 ([M+H]⁺).

### EV301

Using 3,4-dihydro-β-carboline (4) (96.0 mg, 0.564 mmol) and the 5-fluoro-N-methyllisatoic anhydride (7ba) (100.0 mg, 0.512 mmol) under the synthesis conditions for the Evodiamine (1), a pale yellow solid compound, EV301 (125.0 mg, 76% yield) was obtained.

m.p.: 223-224° C.; ¹H-NMR (400 MHz, DMSO-d₆) δ 11.19 (s, 1H), 7.49-7.56 (m, 2H), 7.36-7.41 (m, 2H), 7.20 (q, J=4.4 Hz, 1H), 7.12 (td, J=7.6, 1.3 Hz, 1H), 7.00-7.04 (m, 1H), 6.09 (s, 1H), 4.63 (dt, J=12.6, 3.4 Hz, 1H), 3.17-3.25 (m, 1H), 2.85-2.88 (m, 2H), 2.67 (s, 3H); ¹³C-NMR (100 MHz, DMSO-d₆) δ 163.1, 163.1, 158.4, 156.0, 146.1, 136.7, 129.6, 125.8, 122.2, 122.2, 122.0, 121.9, 121.8, 120.7, 120.5, 118.9, 118.4, 113.6, 113.3, 111.7, 111.7, 69.2, 40.3, 36.6, 19.6; HRMS (FAB) m/z calcd for C₁₉H₁₇FN₃O [M+H]⁺: 322.1356, found 322.1362.

### EV302

:Using 3,4-dihydro-β-carboline (4) (28.5 mg, 0.167 mmol) and 5-methoxy-N-methyllisatoic anhydride (7bb) (31.6 mg, 0.152 mmol) under the synthesis conditions for the Evodiamine (1), a yellow solid compound, EV302 (44.2 mg, 87% yield) was obtained.

m.p.: 222-223° C.; ¹H-NMR (400 MHz, DMSO-d₆) δ 11.25 (s, 1H), 7.51 (d, J=8.2 Hz, 1H), 7.37 (t, J=3.7 Hz, 2H), 7.10-7.18 (m, 3H), 7.00-7.04 (m, 1H), 6.01 (s, 1H), 4.63-4.67 (m, 1H), 3.77 (s, 3H), 3.15-3.22 (m, 1H), 2.78-2.90 (m, 2H), 2.48 (s, 3H); ¹³C-NMR (100 MHz, DMSO-d₆) δ 163.7, 155.0, 143.8, 136.8, 129.5, 125.8, 123.0, 122.9, 121.9, 120.6, 118.9, 118.4, 111.6, 111.6, 110.7, 69.0, 55.4, 36.8, 19.8; LRMS (FAB) m/z, 332 ([M+H]⁺).

### EV303

:Using 3,4-dihydro-β-carboline (4) (283.1 mg, 1.663 mmol) and 5-nitro-N-methylisatoic anhydride (7bc) (336.0 mg, 1.512 mmol) under the synthesis conditions for the Evodiamine (1), a yellow solid compound, EV303 (488.4 mg, 93% yield) was obtained.

m.p.: 290-292° C.; ¹H-NMR (400 MHz, DMSO-d₆) δ 10.90 (s, 1H), 8.45 (d, J=2.8 Hz, 1H), 8.25 (dd, J=9.2, 2.8 Hz, 1H), 7.43 (d, J=7.8 Hz, 1H), 7.34 (d, J=7.8 Hz, 1H), 6.98-7.12 (m, 3H), 6.51 (s, 1H), 4.65 (dd, J=13.2, 5.9 Hz, 1H), 3.46 (s, 3H), 3.30 (dd, J=12.9, 5.1 Hz, 1H), 3.01-3.09 (m, 1H), 2.71 (dd, J=15.4, 4.8 Hz, 1H); ¹³C-NMR (100 MHz, DMSO-d₆) δ 162.8, 151.2, 137.7, 136.1, 131.4, 129.3, 126.2, 124.1, 122.2, 119.2, 118.2, 114.1, 113.4, 111.8, 111.3, 71.3, 42.7, 37.2, 19.6; LRMS (FAB) m/z, 349 ([M+H]⁺).

### EV304

:Using 3,4-dihydro-β-carboline (4) (187.2 mg, 0.363 mmol) and 5-chloro-N-methylisatoic anhydride (7bd) (69.9 mg, 0.330 mmol) under the synthesis conditions for the Evodiamine (1), a yellow solid compound, EV304 (83.9 mg, 75% yield) was obtained.

m.p.: 307-308° C.; ¹H-NMR (400 MHz, DMSO-d₆) δ 11.08 (s, 1H), 7.70 (d, J=2.8 Hz, 1H), 7.51 (dd, J=8.7, 2.3 Hz, 1H), 7.47 (d, J=7.8 Hz, 1H), 7.36 (d, J=7.8 Hz, 1H), 7.07-7.13 (m, 2H), 6.99-7.03 (m, 1H), 6.17 (s, 1H), 4.61 (dd, J=13.1, 4.4 Hz, 1H), 3.22 (td, J=12.2, 4.6 Hz, 1H), 2.88-2.96 (m, 4H), 2.75-2.82 (m, 1H); ¹³C-NMR (100 MHz, DMSO-d₆) δ 163.2, 147.4, 136.5, 133.21, 130.3, 127.1, 126.0, 124.0, 122.0, 120.2, 119.2, 119.0, 118.3, 111.7, 111.6, 69.8, 41.2, 36.4, 19.4; HRMS (FAB) m/z calcd for C₁₉H₁₇ClN₃O [M+H]⁺: 338.1060, found 338.1065.

### EV305

:Using 3,4-dihydro-β-carboline (4) (43.1 mg, 0.253 mmol) and 4-chloro-N-methylisatoic anhydride (7be) (48.7 mg, 0.230 mmol) under the synthesis conditions for the Evodiamine (1), a pale yellow solid compound, EV305 (28.0 mg, 36% yield) was obtained.

m.p.: 291-292° C.; ¹H-NMR (400 MHz, DMSO-d₆) δ 10.94 (s, 1H), 7.71 (d, J=8.3 Hz, 1H), 7.40 (dd, J=37.2, 7.8 Hz, 2H), 7.08-7.12 (m, 1H), 6.98-7.01 (m, 2H), 6.87 (dd, J=8.5, 2.1 Hz, 1H), 6.23 (s, 1H), 4.61 (dd, J=13.1, 5.3 Hz, 1H), 3.19-3.28 (m, 1H), 3.11 (s, 3H), 2.92-3.00 (m, 1H), 2.73 (dd, J=15.4, 4.8 Hz, 1H); ¹³C-NMR (100 MHz, DMSO-d₆) δ 163.8, 148.8, 138.4, 136.3, 131.2, 129.8, 126.1, 122.0, 119.0, 118.8, 118.2, 116.1, 114.7, 111.8, 111.5, 70.6, 41.8, 36.5, 19.3; LRMS (FAB) m/z, 338 ([M+H]⁺).

### EV306

:Using 3,4-dihydro-β-carboline (4) (43.1 mg, 0.253 mmol) and 5-bromo-N-methylisatoic anhydride (7bf) (58.9 mg, 0.230 mmol) under the synthesis conditions for the Evodiamine (1), a pale yellow solid compound, EV306 (60.8 mg, 69% yield) was obtained.

m.p.: 200-201° C.; ¹H-NMR (400 MHz, DMSO-d₆) δ 11.06 (s, 1H), 7.82 (d, J=2.3 Hz, 1H), 7.62 (dd, J=8.7, 2.3 Hz, 1H), 7.47 (d, J=7.8 Hz, 1H), 7.36 (d, J=7.8 Hz, 1H), 7.11 (td, J=7.6, 1.1 Hz, 1H), 6.99-7.03 (m, 2H), 6.18 (s, 1H), 4.61 (dd, J=12.9, 4.6 Hz, 1H), 3.22 (td, J=12.2, 4.6 Hz, 1H), 2.88-2.98 (m, 4H), 2.78 (dd, J=15.6, 4.6 Hz, 1H); ¹³C-NMR (100 MHz, DMSO-d₆) δ 163.13, 147.58, 136.44, 135.97, 130.38, 130.03, 125.96, 121.99, 120.32, 119.17, 119.00, 118.28, 111.70, 111.57, 111.32, 69.86, 41.30, 36.35, 19.40; LRMS (FAB) m/z, 382 ([M+H]⁺).

### EV307

:Using 3,4-dihydro-β-carboline (4) (88.6 mg, 0.520 mmol) and 5-methyl-N-methylisatoic anhydride (7bg) (90.4 mg, 0.473 mmol) under the synthesis conditions for the Evodiamine (1), an orange solid compound, EV307 (49.5 mg, 33% yield) was obtained.

m.p.: 245-247° C.; ¹H-NMR (400 MHz, DMSO-d₆) δ 11.14 (s, 1H), 7.64 (d, J=1.8 Hz, 1H), 7.49 (d, J=7.8 Hz, 1H), 7.30-7.37 (m, 2H), 7.11 (td, J=7.6, 1.1 Hz, 1H), 7.01 (t, J=7.8 Hz, 2H), 6.05 (s, 1H), 4.61-4.66 (m, 1H), 3.14-3.22 (m, 1H), 2.80-2.91 (m, 2H), 2.70 (s, 4H), 2.28 (s, 4H); ¹³C-NMR (100 MHz, DMSO-d₆) δ 164.2, 147.2, 136.6, 134.1, 130.4, 130.2, 128.0, 125.9, 121.9, 120.4, 119.1, 118.9, 118.3, 111.7, 111.6, 69.4, 40.4, 36.6, 20.3, 19.6; HRMS (FAB) m/z calcd for C₂₀H₂ON₃O [M+H]⁺: 318.1606, found 318.1606.

### EV308

:Using 3,4-dihydro-β-carboline (4) (71.7 mg, 0.421 mmol) and 6-chloro-N-methylisatoic anhydride (7bh) (81.1 mg, 0.383 mmol) under the synthesis conditions for the Evodiamine (1), a pale yellow solid compound, EV308 (83.9 mg, 65% yield) was obtained.

m.p.: 267-269° C.; ¹H-NMR (400 MHz, DMSO-d₆) δ 11.29 (s, 1H), 7.55 (d, J=8.3 Hz, 1H), 7.38-7.45 (m, 2H), 7.10-7.18 (m, 3H), 7.02-7.06 (m, 1H), 5.94 (s, 1H), 4.49 (dq, J=12.8, 2.6 Hz, 1H), 3.25-3.31 (m, 1H), 2.81-2.94 (m, 2H), 2.65 (s, 3H); ¹³C-NMR (100 MHz, DMSO-d₆) δ 161.3, 152.3, 136.8, 134.1, 132.8, 128.4, 125.6, 124.4, 122.2, 119.0, 118.7, 118.6, 111.9, 111.7, 67.4, 35.7, 19.7; LRMS (FAB) m/z, 338 ([M+H]⁺).

### EV309

:Using 3,4-dihydro-β-carboline (4) (67.8 mg, 0.398 mmol) and 6-methoxy-N-methylisatoic anhydride (7bi) (75.0 mg, 0.362 mmol) under the synthesis conditions for the Evodiamine (1), a yellow solid compound, EV309 (54.8 mg, 45% yield) was obtained.

m.p.: 248-250° C.; ¹H-NMR (400 MHz, DMSO-d₆) δ 11.23 (s, 1H), 7.52 (d, J=7.8 Hz, 1H), 7.36-7.41 (m, 2H), 7.13 (td, J=7.6, 1.1 Hz, 1H), 7.01-7.05 (m, 1H), 6.73 (d, J=8.3 Hz, 1H), 6.67 (d, J=7.8 Hz, 1H), 5.83 (s, 1H), 4.44-4.49 (m, 1H), 3.77 (s, 3H), 3.20-3.27 (m, 1H), 2.78-2.89 (m, 2H), 2.62 (s, 3H); ¹³C-NMR (100 MHz, DMSO-d₆) b 162.0, 160.5, 152.0, 136.8, 133.2, 129.1, 125.7, 122.0, 118.9, 118.5, 111.8, 111.7, 111.5, 110.7, 105.4, 67.7, 55.8, 35.8, 19.8; LRMS (FAB) m/z, 334 ([M+H]⁺).

### EV310

:Using 3,4-dihydro-β-carboline (4) (31.6 mg, 0.186 mmol) and 5-iodo-N-methylisatoic anhydride (7bj) (51.2 mg, 0.169 mmol) under the synthesis conditions for the Evodiamine (1), a pale yellow solid compound, EV310 (40.4 mg, 56% yield) was obtained.

m.p.: 293-295° C.; ¹H-NMR (400 MHz, DMSO-d₆) δ 11.03 (s, 1H), 7.98 (d, J=2.3 Hz, 1H), 7.74 (dd, J=8.7, 2.3 Hz, 1H), 7.46 (d, J=7.8 Hz, 1H), 7.35 (d, J=8.3 Hz, 1H), 7.09-7.13 (m, 1H), 6.99-7.02 (m, 1H), 6.87 (d, J=8.7 Hz, 1H), 6.17 (s, 1H), 4.60 (dd, J=13.0, 4.5 Hz, 1H), 3.21 (td, J=12.2, 4.7 Hz, 1H), 2.96 (s, 3H), 2.88-2.94 (m, 1H), 2.77 (dd, J=15.6, 4.6 Hz, 1H); ¹³C-NMR (100 MHz, DMSO-d₆) δ 163.1, 147.8, 141.6, 136.4, 136.0, 130.6, 126.0, 122.0, 120.4, 119.1, 119.0, 118.3, 111.7, 111.5, 81.9, 69.9, 41.4, 36.3, 19.4; LRMS (FAB) m/z, 430 ([M+H]⁺).

### EV311

:Using 3,4-dihydro-β-carboline (4) (45.5 mg, 0.267 mmol) and 4-methoxy-N-methylisatoic anhydride (7bk) (50.3 mg, 0.243 mmol) under the synthesis conditions for the Evodiamine (1), a pale yellow solid compound, EV311 (44.2 mg, 55% yield) was obtained.

m.p.: 246-248° C.; ¹H-NMR (400 MHz, DMSO-d₆) δ 10.97 (s, 1H), 7.68 (d, J=8.7 Hz, 1H), 7.45 (d, J=7.8 Hz, 1H), 7.35 (d, J=8.3 Hz, 1H), 7.08-7.12 (m, 1H), 6.98-7.02 (m, 1H), 6.44-6.50 (m, 2H), 6.13 (s, 1H), 4.60 (dd, J=12.9, 5.1 Hz, 1H), 3.81 (s, 3H), 3.18 (td, J=12.2, 4.7 Hz, 1H), 3.00 (s, 3H), 2.88-2.96 (m, 1H), 2.73 (dd, J=15.2, 4.6 Hz, 1H); ¹³C-NMR (100 MHz, DMSO-d₆) δ 164.4, 163.7, 150.0, 136.4, 131.3, 129.9, 126.1, 121.8, 118.9, 118.2, 111.7, 111.6, 111.4, 106.5, 100.4, 70.4, 55.4, 41.2, 36.5, 19.4; LRMS (FAB) m/z, 334 ([M+H]⁺).

### EV312

:Using 3,4-dihydro-β-carboline (4) (58.4 mg, 0.343 mmol) and 3-methyl-N-methylisatoic anhydride (7bl) (59.6 mg, 0.312 mmol) under the synthesis conditions for the Evodiamine (1), an orange solid compound, EV312 (84.1 mg, 85% yield) was obtained.

m.p.: 256-258° C.; ¹H-NMR (400 MHz, CDCl₃) δ 8.31 (s, 1H), 7.99-8.01 (m, 1H), 7.61 (d, J=7.8 Hz, 1H), 7.41 (dd, J=28.3, 7.6 Hz, 2H), 7.27-7.30 (m, 1H), 7.13-7.23 (m, 3H), 5.88 (s, 1H), 4.90 (dq, J=12.9, 2.5 Hz, 1H), 3.29-3.36 (m, 1H), 2.91-3.03 (m, 2H), 2.41 (s, 3H), 2.26 (s, 3H); ¹³C-NMR (100 MHz, DMSO-d₆) δ 163.7, 149.1, 137.0, 134.4, 132.3, 128.8, 125.8, 125.8, 124.8, 124.5, 122.0, 118.8, 118.4, 111.7, 111.6, 68.8, 33.8, 19.8, 16.5; LRMS (FAB) m/z, 318 ([M+H]⁺).

### Synthesis Example 5. Synthesis of A-Ring Evodiamine Derivatives (EV401˜413)

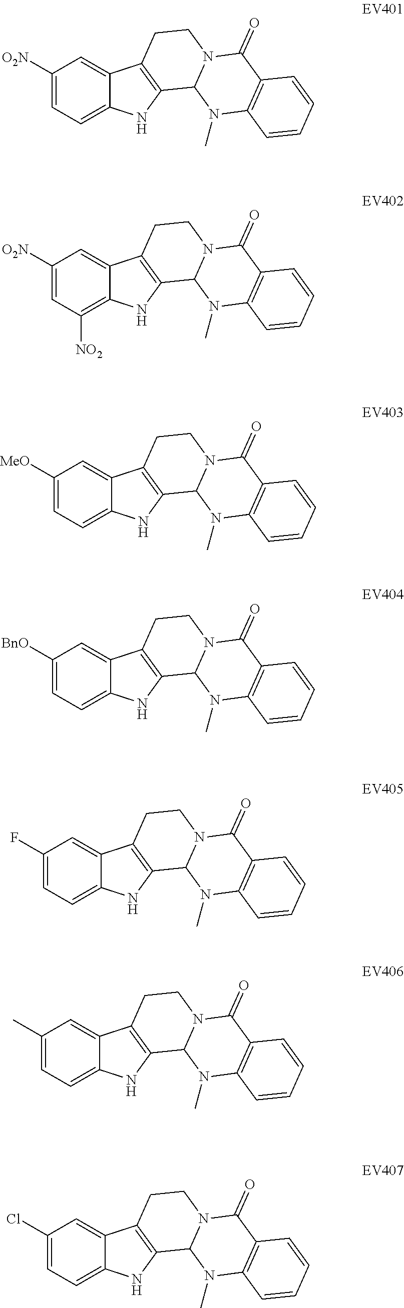

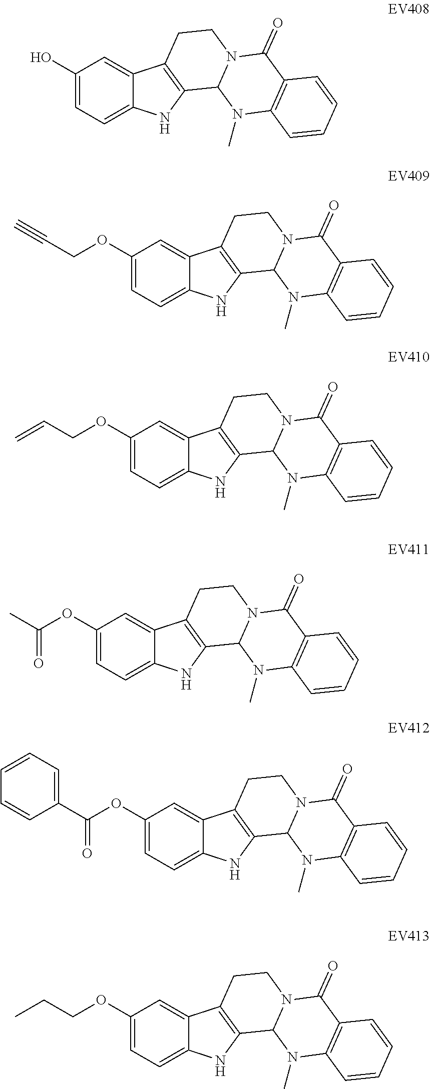 [SMILES: CN1c2ccccc2C(=O)N2CCc3c([nH]c4ccc(O)cc34)C21 ; C#CCOc1ccc2[nH]c3c(c2c1)CCN1C(=O)c2ccccc2N(C)C31 ; C=CCOc1ccc2[nH]c3c(c2c1)CCN1C(=O)c2ccccc2N(C)C31 ; CC(=O)Oc1ccc2[nH]c3c(c2c1)CCN1C(=O)c2ccccc2N(C)C31 ; CN1c2ccccc2C(=O)N2CCc3c([nH]c4ccc(OC(=O)c5ccccc5)cc34)C21 ; CCCOc1ccc2[nH]c3c(c2c1)CCN1C(=O)c2ccccc2N(C)C31]

### EV401

:Upon addition of KNO₃ (33.4 mg, 0.330 mmol) and concentrated H₂SO₄ (1.0 mL, 0.33 M) to Evodiamine (1) (100.0 mg, 0.330 mmol), the mixture was stirred overnight at 0° C. After completion of the reaction, ice water (10 mL) was added to the mixture and the resultant solid was obtained by vacuum filtration, yielding an yellow solid compound EV401 (111.5 mg, 97% yield).

m.p.: 220-222° C.; ¹H-NMR (400 MHz, DMSO-d₆) δ 11.89 (s, 1H), 8.51 (s, 1H), 8.02 (dd, J=9.0, 2.1 Hz, 1H), 7.79-7.87 (m, 1H), 7.47-7.53 (m, 2H), 7.07 (d, J=8.3 Hz, 1H), 6.98 (t, J=7.6 Hz, 1H), 6.21 (s, 1H), 4.62-4.66 (m, 1H), 3.20-3.28 (m, 1H), 2.91-2.95 (m, 5H); ¹³C-NMR (100 MHz, DMSO-D₆) δ 164.2, 148.6, 140.6, 139.6, 134.9, 133.6, 128.0, 125.4, 120.4, 119.2, 117.7, 117.3, 115.6, 114.3, 112.1, 69.6, 40.7, 36.9, 19.2; HRMS (FAB) m/z calcd for C₁₉H₁₇N₄O₃ [M+H]⁺: 349.1301, found 349.1294.

### EV402

:Upon addition of KNO₃ (66.8 mg, 0.660 mmol) and concentrated H₂SO₄ (1.0 mL, 0.33 M) to Evodiamine (1) (100.0 mg, 0.330 mmol) at 0° C., the mixture was stirred overnight at room temperature. After completion of the reaction, ice water (10 mL) was added to the mixture and the resultant solid was purified by column chromatography (silica gel, hexane:ethyl acetate=2:1˜1:1) to yield a yellow solid compound EV402, (14.3 mg, 11% yield).

m.p.: 268-270° C.; ¹H-NMR (400 MHz, DMSO-d₆) δ 12.63 (s, 1H), 9.03 (d, J=2.0 Hz, 1H), 8.80 (d, J=2.0 Hz, 1H), 7.88 (dd, J=7.4, 1.3 Hz, 1H), 7.51-7.56 (m, 1H), 7.22 (d, J=8.1 Hz, 1H), 7.11 (t, J=7.2 Hz, 1H), 6.04 (s, 1H), 4.68 (dd, J=12.1, 4.7 Hz, 1H), 3.10-3.21 (m, 2H), 2.85-2.92 (m, 1H), 2.63 (s, 3H); ¹³C-NMR (100 MHz, DMSO-d₆) b 163.6, 149.9, 139.5, 135.7, 133.2, 131.8, 131.3, 129.7, 128.2, 122.1, 121.8, 121.2, 119.7, 117.3, 114.4, 67.7, 40.4, 39.9, 39.7, 39.5, 39.3, 39.2, 39.1, 38.9, 36.0, 19.7; HRMS (FAB) m/z calcd for C₁₉H₁₆N₅O₅ [M+H]⁺: 394.1151, found 394.1157.

### 5-1. Method for Synthesis of EV403, EV405-407 Derivatives

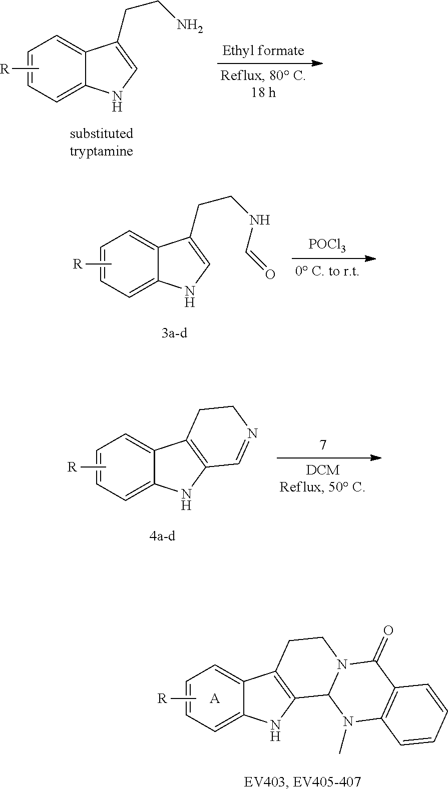

### 5-Fluoro-N-formyltryptamine (3b)

:Following the synthesis conditions for the N-formyltryptamine (3), 5-fluorotryptamine (165.9 mg, 0.931 mmol) and ethyl formate (344.8 mg, 4.655 mmol) were used to obtain the brown oil compound 3b (192.0 mg, 99% yield).

¹H-NMR (400 MHz, CDCl₃) δ 8.32 (s, 1H), 8.12 (s, 1H), 6.90-7.29 (m, 3H), 5.83 (d, J=91.9 Hz, 1H), 3.61 (q, J=6.6 Hz, 2H), 2.89-2.95 (m, 2H); ¹³C-NMR (100 MHz, CDCl₃) δ 161.3, 124.0, 112.1, 112.0, 111.0, 110.8, 103.8, 103.6, 38.3, 25.3; LRMS (FAB) m/z, 303 ([M+H]⁺), 273, 235. LRMS (FAB) m/z, 207 ([M+H]⁺).

### 6-fluoro-4,9-dihydro-3H-pyrido[3,4-b]indole (4b)

:Following the synthesis conditions for the 3,4-Dihydro-p-carboline (4), 3b (313.1 mg, 1.518 mmol) and POCl₃ (698.2 mg, 4.554 mmol) were used to obtain the yellow solid compound 4b (200.0 mg, 70% yield).

m.p.: 150-152° C.; ¹H-NMR (400 MHz, CDCl₃) δ 9.19 (s, 1H), 7.90 (s, 1H), 7.26 (t, J=6.7 Hz, 1H), 7.07 (dd, J=9.2, 2.3 Hz, 1H), 6.95 (td, J=9.0, 2.5 Hz, 1H), 4.18 (t, J=8.5 Hz, 2H), 3.10 (t, J=8.5 Hz, 2H); ¹³C-NMR (100 MHz, CDCl₃) δ 156.9, 151.4, 134.8, 129.6, 125.7, 116.5, 113.2, 100.8, 58.0, 49.6, 19.1; LRMS (FAB) m/z, 189 ([M+H]⁺).

### EV403

:Following the synthesis conditions for the Evodiamine (1), N-methylisatoic anhydride (7) (50.1 mg, 0.283 mmol) and 4a (62.3 mg, 0.311 mmol) were used to obtain the pale yellow solid compound EV403 (56.6 mg, 60% yield).

m.p.: 312-313° C.; ¹H-NMR (400 MHz, DMSO-d₆) δ 10.88 (s, 1H), 7.79 (dd, J=7.8, 1.8 Hz, 1H), 7.45-7.49 (m, 1H), 7.24 (d, J=8.7 Hz, 1H), 7.03 (d, J=7.8 Hz, 1H), 6.93-6.97 (m, 2H), 6.74 (dd, J=8.7, 2.3 Hz, 1H), 6.10 (s, 1H), 4.60-4.64 (m, 1H), 3.75 (s, 3H), 3.19 (td, J=12.2, 4.3 Hz, 1H), 2.84-2.92 (m, 4H), 2.75 (dd, J=15.2, 4.6 Hz, 1H); ¹³C-NMR (100 MHz, DMSO-d₆) δ 164.3, 153.4, 148.7, 133.5, 131.5, 131.3, 128.0, 126.3, 120.2, 119.2, 117.3, 112.4, 112.0, 111.3, 100.0, 69.9, 55.4, 41.0, 36.4, 19.6; LRMS (FAB) m/z, 334 ([M+H]⁺).

### EV405

:Following the synthesis conditions for the Evodiamine (1), N-methylisatoic anhydride (7) (17.4 mg, 0.098 mmol) and 4b (20.3 mg, 1.108 mmol) were used to obtain the pale yellow solid compound EV405 (17.4 mg, 55% yield).

m.p.: 313-314° C.; ¹H-NMR (400 MHz, DMSO-d₆); δ 11.15 (s, 1H), 7.77 (dd, J=7.6, 1.5 Hz, 1H), 7.47-7.49 (m, 1H), 7.35 (d, J=7.9 Hz, 1H), 7.22 (dd, J=9.8, 2.4 Hz, 1H), 7.04 (d, J=7.9 Hz, 1H), 6.91-6.96 (m, 2H), 6.13 (s, 1H), 4.62 (dd, J=12.8, 4.9 Hz, 1H), 3.20 (td, J=12.2, 4.7 Hz, 1H), 2.91 (s, 3H), 2.70-2.77 (m, 1H); ¹³C-NMR (100 MHz, DMSO-d₆) δ 164.2, 148.6, 133.6, 133.0, 131.6, 128.0, 126.3, 123.6, 120.2, 118.9, 117.2, 114.0, 111.7, 110.7, 110.0, 69.9, 41.0, 36.6, 19.4; LRMS (FAB) m/z, 322 ([M+H]⁺).

### EV406

:Following the synthesis conditions for the Evodiamine (1), N-methylisatoic anhydride (7) (47.3 mg, 0.267 mmol) and 4c (54.1 mg, 0.294 mmol) were used to obtain the pale yellow solid compound EV406 (16.1 mg, 19% yield).

m.p.: 284-285° C.; ¹H-NMR (400 MHz, DMSO-d₆) δ 10.91 (s, 1H), 7.78 (dd, J=7.8, 1.4 Hz, 1H), 7.47 (td, J=7.7, 1.5 Hz, 1H), 7.24 (d, J=8.3 Hz, 2H), 7.04 (d, J=7.8 Hz, 1H), 6.92-6.97 (m, 2H), 6.10 (s, 1H), 4.62 (dd, J=12.9, 4.6 Hz, 1H), 3.18 (td, J=12.1, 4.4 Hz, 1H), 2.84-2.92 (m, 4H), 2.75 (dd, J=15.2, 4.6 Hz, 1H), 2.36 (s, 3H); ¹³C-NMR (100 MHz, DMSO-d₆) δ 164.3, 148.8, 134.9, 133.5, 130.7, 128.0, 127.4, 126.2, 123.5, 120.2, 119.2, 117.9, 117.4, 111.4, 111.0, 69.8, 40.9, 36.4, 21.2, 19.5; LRMS (FAB) m/z, 318 ([M+H]⁺).

### EV407

:Following the synthesis conditions for the Evodiamine (1), N-methylisatoic anhydride (7) (24.6 mg, 0.139 mmol) and 4c (31.3 mg, 0.153 mmol) were used to obtain the pale yellow solid compound EV407 (18.5 mg, 39% yield).

m.p.: 312-313° C.; ¹H-NMR (400 MHz, DMSO-d₆) δ 11.27 (s, 1H), 7.78 (d, J=7.8 Hz, 1H), 7.53 (d, J=2.1 Hz, 1H), 7.45-7.50 (m, 1H), 7.36 (d, J=8.7 Hz, 1H), 7.10 (dd, J=8.5, 2.1 Hz, 1H), 7.04 (d, J=8.3 Hz, 1H), 6.95 (t, J=7.6 Hz, 1H), 6.15 (s, 1H), 4.61 (dd, J=12.9, 5.1 Hz, 1H), 3.20 (td, J=12.2, 4.9 Hz, 1H), 2.90 (s, 3H), 2.85-2.93 (m, 1H), 2.77 (dd, J=15.4, 4.4 Hz, 1H); ¹³C-NMR (100 MHz, DMSO-d₆) δ 164.2, 148.6, 134.9, 133.6, 132.8, 128.0, 127.2, 123.6, 121.8, 120.3, 119.0, 117.7, 117.4, 113.2, 111.4, 69.79, 40.9, 36.7, 19.3; LRMS (FAB) m/z, 338 ([M+H]⁺).

### 5-2. Method for Synthesis of EV408 Derivative

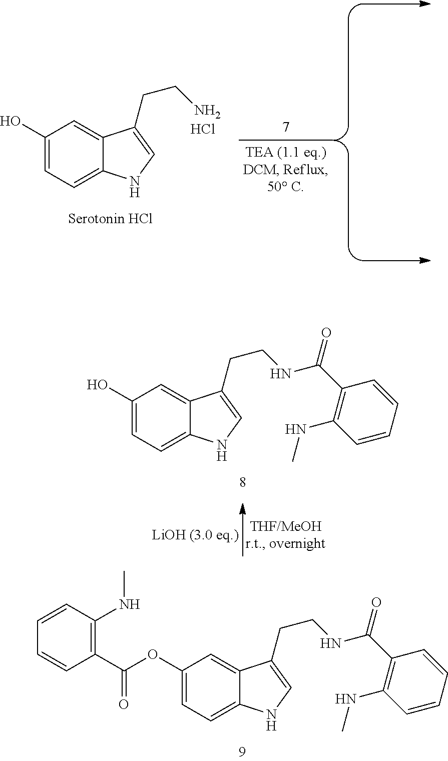 [SMILES: NCCc1c[nH]c2ccc(O)cc12 ; Cl ; CNc1ccccc1C(=O)NCCc1c[nH]c2ccc(O)cc12 ; CNc1ccccc1C(=O)NCCc1c[nH]c2ccc(OC(=O)c3ccccc3NC)cc12]

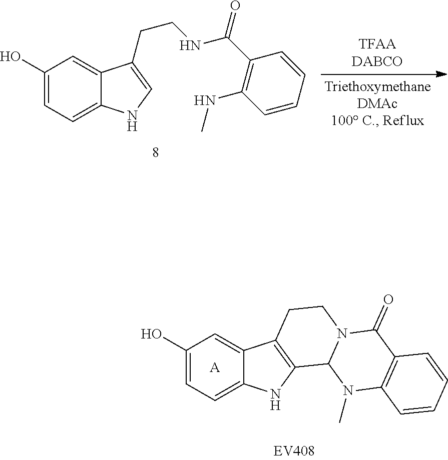

### General Synthesis Method for Intermediates 8, 9

:Serotonin hydrochloride (100.0 mg, 0.470 mmol) and N-methylisatoic anhydride (7) (75.7 mg, 0.427 mmol) were dissolved in CH₂Cl₂ (1.1 mL, 0.4 M), then triethylamine (43.3 mg, 0.427 mmol) was added. The temperature was then raised to 50° C., and the mixture was refluxed with stirring for 3 hours. After the reaction was complete, excess CH₂C₁₂ was removed by vacuum concentration, and the resulting mixture was purified by column chromatography (silica gel, hexane:ethyl acetate=2:1˜1:1) to obtain colorless oil compound 8 (33.1 mg, 22% yield), and white solid compound 9 (68.9 mg, 33% yield).

### General Conditions for Hydrolysis of Intermediate 9

:Intermediate 9 (68.9 mg, 0.156 mmol) was dissolved in THF (2.3 mL, 0.06 M), then LiOH (11.2 mg, 0.467 mmol) was dissolved in MeOH (2.3 mL, 0.06 M) and slowly added to the reaction mixture. The mixture was stirred at room temperature for 12 hours, then vacuum concentration was used to remove excess THF and MeOH. The resulting mixture was dissolved in ethyl acetate (30 mL), and the organic phase was washed with water 3 times (10 mL×3). The aqueous phase was then extracted with ethyl acetate 3 times (10 mL×3). The obtained organic phase was dried and filtered with MgSO₄, then concentrated under vacuum. The resulting mixture was purified by column chromatography (silica gel, hexane:ethyl acetate=2:1˜1:1) to obtain colorless oil compound 8 (48.2 mg, 99.9% yield).

### Intermediate 8

:¹H-NMR (400 MHz, CDCl₃) δ 7.92 (d, J=16.8 Hz, 1H), 7.39 (s, 1H), 7.27-7.31 (m, 1H), 7.20 (d, J=8.7 Hz, 1H), 7.14 (dd, J=7.8, 1.4 Hz, 1H), 7.00 (s, 3H), 6.78 (dd, J=8.6, 2.4 Hz, 1H), 6.67 (d, J=8.3 Hz, 1H), 6.52 (t, J=7.5 Hz, 1H), 6.16 (s, 1H), 5.29 (d, J=4.1 Hz, 1H), 3.67 (q, J=6.4 Hz, 3H), 2.98 (t, J=6.7 Hz, 3H), 2.85 (s, 4H); ¹³C-NMR (100 MHz, CDCl₃) δ 171.5, 170.3, 150.4, 149.9, 132.9, 131.6, 128.1, 127.4, 123.3, 115.8, 115.0, 112.4, 112.3, 112.1, 111.4, 103.3, 40.2, 29.9, 25.3; LRMS (FAB) m/z, 310 ([M+H]⁺).

### Intermediate 9

:m.p.: 81-83° C.; ¹H-NMR (400 MHz, CDCl₃) δ 8.18 (dd, J=8.0, 1.6 Hz, 1H), 8.13 (s, 1H), 7.66 (d, J=5.1 Hz, 1H), 7.44-7.48 (m, 2H), 7.40 (d, J=2.1 Hz, 1H), 7.37 (d, J=8.7 Hz, 1H), 7.26-7.30 (m, 2H), 7.16 (dd, J=7.8, 1.6 Hz, 1H), 7.07 (d, J=2.3 Hz, 1H), 7.01 (dd, J=8.7, 2.1 Hz, 1H), 6.73 (d, J=8.5 Hz, 1H), 6.66-6.70 (m, 1H), 6.62-6.64 (m, 1H), 6.50-6.54 (m, 1H), 6.11 (s, 1H), 3.71 (q, J=6.4 Hz, 2H), 3.03 (t, J=6.7 Hz, 2H), 2.92 (d, J=5.1 Hz, 3H), 2.84 (d, J=4.4 Hz, 3H); ¹³C-NMR (100 MHz, CDCl₃) δ 170.0, 168.5, 152.7, 150.4, 144.3, 135.4, 134.4, 132.8, 132.2, 127.8, 127.3, 123.6, 116.9, 115.5, 114.8, 114.6, 113.4, 111.9, 111.3, 111.0, 109.3, 39.9, 29.9, 29.7, 25.4; LRMS (FAB) m/z, 443 ([M+H]⁺).

### EV408

:Intermediate 8 (1.0 equiv.) was dissolved in triehoxymethane-DMF (0.33 M), then trifluoroacetic anhydride (1.0 equiv.) and DABCO (1.1 equiv.) were added sequentially. The temperature of the reaction mixture was then raised to 100° C., and the mixture was refluxed with stirring for 6 hours. After the reaction was complete, the mixture was dissolved in ethyl acetate (30 mL), and the organic phase was washed with water 3 times (10 mL×3). The aqueous phase was then extracted with ethyl acetate 3 times (10 mL×3). The obtained organic phase was dried and filtered with MgSO₄, then concentrated under vacuum. The resulting mixture was purified by column chromatography (silica gel, hexane:ethyl acetate=2:1-1:1) to obtain white solid compound EV408 (40.0 mg, 41% yield).

m.p.: 276-277° C.; ¹H-NMR (400 MHz, DMSO-d₆) δ 10.71 (s, 1H), 8.70 (s, 1H), 7.78 (dd, J=8.1, 1.3 Hz, 1H), 7.45-7.49 (m, 1H), 7.14 (d, J=8.8 Hz, 1H), 7.02 (d, J=8.1 Hz, 1H), 6.94 (t, J=7.4 Hz, 1H), 6.75 (d, J=1.7 Hz, 1H), 6.62 (dd, J=8.4, 2.4 Hz, 1H), 6.08 (s, 1H), 4.60 (dd, J=12.8, 4.7 Hz, 1H), 3.18 (td, J=12.1, 4.5 Hz, 1H), 2.90 (s, 3H), 2.80-2.87 (m, 1H), 2.66 (dd, J=15.5, 4.0 Hz, 1H); ¹³C-NMR (100 MHz, DMSO-d₆) δ 164.3, 150.7, 148.7, 133.5, 131.2, 130.9, 128.0, 126.7, 120.0, 119.0, 117.1, 112.1, 112.1, 110.6, 102.1, 70.0, 41.1, 36.4, 19.6; HRMS (FAB) m/z calcd for C₁₉H₁₈N₃O₂ [M+H]⁺: 320.1399, found 320.1401.

### 5-3. Method for Synthesis of EV404, EV409-413 Derivatives

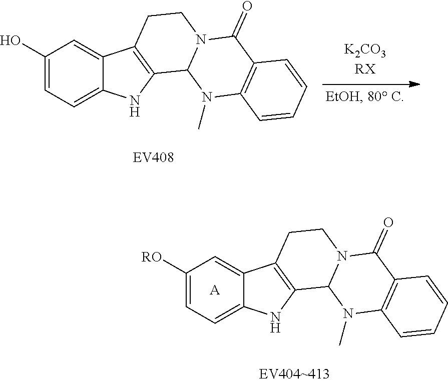

### General Conditions for O-alkylation

EV408 (1.0 equiv.) and K₂CO₃ (1.2 equiv.) were dissolved in EtOH (0.05 M), followed by the addition of alkyl halide (1.2 equiv.). The temperature of the reaction mixture was then raised to 80° C. and refluxed with stirring overnight. After the completion of the reaction, the mixture was concentrated under vacuum to remove the excess EtOH. The obtained mixture was dissolved in ethyl acetate (30 mL), and the organic phase was washed with water three times (10 mL×3). The aqueous phase was extracted with ethyl acetate three times (10 mL×3) again. The obtained organic phase was dried and filtered with MgSO₄, then concentrated under vacuum. The obtained mixture was purified by column chromatography (silica gel, hexane:ethyl acetate=3:1˜1:1) to yield solid compounds EV404 and EV409-413.

### EV404

Under the general conditions for O-alkylation, using EV408 (14.8 mg, 0.046 mmol) and benzyl bromide (9.4 mg, 0.055 mmol), a yellow solid compound, EV404 (8.7 mg, 46% yield), was obtained.

m.p.: 188-190° C.; ¹H-NMR (400 MHz, DMSO-d₆) δ 8.13 (s, 1H), 8.10 (m, 1H), 7.46 (m, 1H), 7.37 (t, J=7.5 Hz, 1H), 7.30 (m, 1H), 7.17 (t, J=7.5 Hz, 1H), 7.10 (m, 1H), 6.97 (dd, J=8.5 Hz, 1H), 5.87 (s, 1H), 5.11 (s, 1H), 4.84 (m, 1H), 3.25 (m, 1H), 2.92 (m, 2H), 2.49 (s, 3H); ¹³C-NMR (100 MHz, CDCl₃) δ 164.8, 153.7, 150.7, 137.6, 133.1, 132.0, 129.2, 129.0, 128.6, 127.9, 127.6, 126.7, 124.0, 123.7, 122.3, 114.0, 113.5, 112.1, 102.5, 71.0, 69.0, 39.6, 37.3, 20.2; LRMS (FAB) m/z, 410 ([M+H]⁺).

### EV409

:Under the general conditions for O-alkylation, using EV408 (32.2 mg, 0.101 mmol) and propargyl bromide (14.4 mg, 0.121 mmol), a yellow solid compound, EV409 (31.7 mg, 88% yield), was obtained.

m.p.: 170-172° C.; ¹H-NMR (400 MHz, CDCl₃) δ 8.19 (s, 1H), 8.11 (dd, J=7.8, 1.4 Hz, 1H), 7.48 (td, J=7.7, 1.5 Hz, 1H), 7.33 (t, J=8.2 Hz, 1H), 7.19-7.23 (m, 1H), 7.13 (q, J=2.5 Hz, 2H), 6.98 (dd, J=8.7, 2.3 Hz, 1H), 5.89 (s, 1H), 4.87 (dt, J=12.7, 3.7 Hz, 1H), 4.75 (d, J=2.3 Hz, 2H), 3.22-3.30 (m, 1H), 2.92-2.95 (m, 2H), 2.51-2.53 (m, 4H); ¹³C-NMR (100 MHz, DMSO-d₆) δ 164.3, 151.3, 148.7, 133.5, 132.0, 131.6, 128.0, 126.2, 120.2, 119.2, 117.4, 112.5, 112.4, 111.4, 102.1, 80.0, 77.8, 69.8, 56.1, 41.1, 36.5, 19.6; LRMS (FAB) m/z, 360 ([M+H]⁺).

### EV410

:Under the general conditions for O-alkylation, using EV408 (20.0 mg, 0.063 mmol) and allyl bromide (11.4 mg, 0.094 mmol), a yellow solid compound, EV410 (21.9 mg, 96.9% yield), was obtained.

m.p.: 176-178° C.; ¹H-NMR (400 MHz, CDCl₃) δ 8.13 (s, 1H), 8.08 (d, J=7.0 Hz, 1H), 7.44 (m, 1H), 7.26 (d, J=6.5 Hz, 1H), 7.17 (t, J=7.5 Hz, 1H), 7.10 (d, J=8.0 Hz, 1H), 7.01 (s, 1H), 6.92 (dd, J=8.0 Hz, 1H), 6.10 (m, 1H), 5.86 (s, 1H), 5.43 (dd, J=18 Hz, 1H), 5.27 (dd, J=10 Hz, 1H), 4.84 (m, 1H), 4.58 (d, J=5.5 Hz, 2H), 3.26 (m, 1H), 2.90 (m, 2H), 2.49 (s, 3H); ¹³C-NMR (100 MHz, CDCl₃) δ 164.7, 153.4, 150.6, 133.8, 133.0, 131.9, 129.1, 129.0, 126.6, 124.0, 123.6, 122.2, 117.4, 113.9, 113.4, 112.0, 102.3, 69.8, 68.9, 39.6, 37.2, 20.1; LRMS (FAB) m/z, 358 ([M+H]⁺).

### EV411

:Under the general conditions for O-alkylation, using EV408 (20.0 mg, 0.063 mmol) and benzoyl chloride (13.2 mg, 0.094 mmol), a yellow solid compound, EV411 (19.2 mg, 72% yield), was obtained.

m.p.: 224-226° C.; ¹H-NMR (400 MHz, DMSO-d₆) δ 10.77 (s, 1H), 7.82 (s, 1H), 7.77 (dd, J=7.8, 1.4 Hz, 1H), 7.44-7.48 (m, 1H), 7.23 (s, 1H), 7.14 (d, J=8.7 Hz, 1H), 7.01 (d, J=7.8 Hz, 1H), 6.93 (t, J=7.4 Hz, 1H), 6.75 (d, J=1.8 Hz, 1H), 6.63 (dd, J=8.7, 2.3 Hz, 1H), 6.08 (s, 1H), 4.60 (dd, J=12.6, 4.8 Hz, 1H), 3.17 (td, J=12.2, 4.4 Hz, 1H), 2.90 (s, 3H), 2.79-2.86 (m, 1H), 2.67 (td, J=15.3, 4.3 Hz, 1H); ¹³C-NMR (100 MHz, DMSO-d₆) δ 164.2, 150.8, 148.6, 139.0, 133.5, 131.2, 130.9, 128.9, 127.9, 126.9, 126.7, 120.0, 118.9, 117.0, 112.1, 110.5, 102.1, 70.0, 41.1, 36.4, 28.2, 19.6; LRMS (FAB) m/z, 424 ([M+H]⁺).

### EV412

:Under the general conditions for O-alkylation, using EV408 (20.0 mg, 0.063 mmol) and acetyl chloride (7.4 mg, 0.094 mmol), a yellow solid compound, EV412 (12.5 mg, 55% yield), was obtained.

m.p.: 201-203° C.; ¹H-NMR (400 MHz, CDCl₃) δ 8.29 (s, 1H), 8.09 (m, 1H), 7.46 (m, 1H), 7.26 (d, J=2.0 Hz, 1H), 7.20 (m, 1H), 7.11 (d, J=8.0 Hz, 1H), 6.94 (dd, J=8.5 Hz, 1H), 5.87 (s, 1H), 5.11 (s, 1H), 4.82 (m, 1H), 3.25 (m, 1H), 2.88 (m, 2H), 2.49 (s, 3H), 2.31 (s, 3H); ¹³C-NMR (100 MHz, CDCl₃) δ 170.5, 164.7, 150.5, 144.5, 134.5, 133.1, 129.8, 129.0, 126.6, 124.1, 123.6, 122.3, 117.1, 113.9, 111.8, 111.2, 68.8, 39.5, 37.3, 21.2, 20.1; LRMS (FAB) m/z, 362 ([M+H]⁺).

### EV413

:Under the general conditions for O-alkylation, using EV408 (28.0 mg, 0.088 mmol) and 1-chloropropane (16.2 mg, 0.132 mmol), a yellow solid compound, EV413 (14.0 mg, 44% yield), was obtained.

m.p.: 158-160° C.; ¹H-NMR (400 MHz, CDCl₃) δ 0.89-1.08 (t, J=7.0 Hz, 3H), 1.84-1.86 (m, 2H), 2.50 (s, 3H), 2.92-2.93 (m, 2H), 3.25-3.31 (m, 1H), 3.99 (t, J=6.6 Hz, 2H), 4.84-4.88 (m, 1H), 5.90 (s, 1H), 6.92 (d, J=8.8 Hz, 1H), 7.03 (s, 1H), 7.13 (d, J=8.0 Hz, H), 7.20 (t, J=7.7 Hz, 1H), 7.29 (d, J=8.8 Hz, 1H), 7.48 (t, J=8.0 Hz, 1H), 8.08 (s, 1H), 8.11 (d, J=7.7 Hz, 1H). ¹³C-NMR (100 MHz, CDCl₃) δ 164.3 153.4, 150.2, 132.6, 131.3, 128.5 (2C), 126.2, 123.4, 123.1, 121.7, 113.3, 112.9, 111.6, 101.4, 70.0, 68.4, 39.2, 36.7, 22.3, 19.7, 10.2; LRMS (FAB) m/z, 362 ([M+H]⁺).

### Synthesis Example 6. Synthesis of Multiple Substituted Evodiamine Derivatives (EV501˜509)

### Synthesized Evodiamine Derivatives EV501˜509

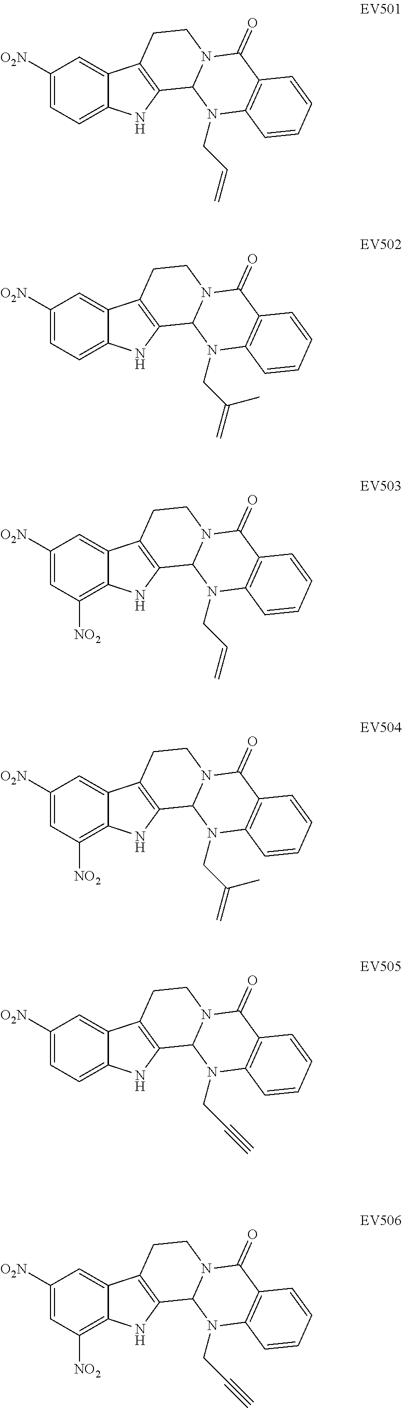 [SMILES: C=CCN1c2ccccc2C(=O)N2CCc3c([nH]c4ccc([N+](=O)[O-])cc34)C21 ; C=C(C)CN1c2ccccc2C(=O)N2CCc3c([nH]c4ccc([N+](=O)[O-])cc34)C21 ; C=CCN1c2ccccc2C(=O)N2CCc3c([nH]c4c([N+](=O)[O-])cc([N+](=O)[O-])cc34)C21 ; C=C(C)CN1c2ccccc2C(=O)N2CCc3c([nH]c4c([N+](=O)[O-])cc([N+](=O)[O-])cc34)C21 ; C#CCN1c2ccccc2C(=O)N2CCc3c([nH]c4ccc([N+](=O)[O-])cc34)C21 ; C#CCN1c2ccccc2C(=O)N2CCc3c([nH]c4c([N+](=O)[O-])cc([N+](=O)[O-])cc34)C21]

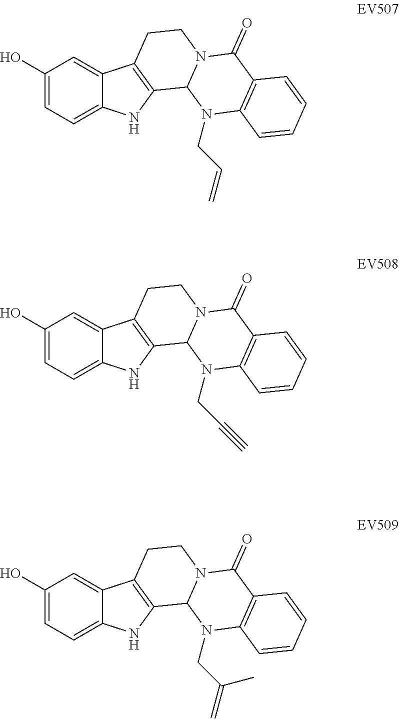 [SMILES: C=CCN1c2ccccc2C(=O)N2CCc3c([nH]c4ccc(O)cc34)C21 ; C#CCN1c2ccccc2C(=O)N2CCc3c([nH]c4ccc(O)cc34)C21 ; C=C(C)CN1c2ccccc2C(=O)N2CCc3c([nH]c4ccc(O)cc34)C21]

### 6-1. Method for Synthesis of EV501-506

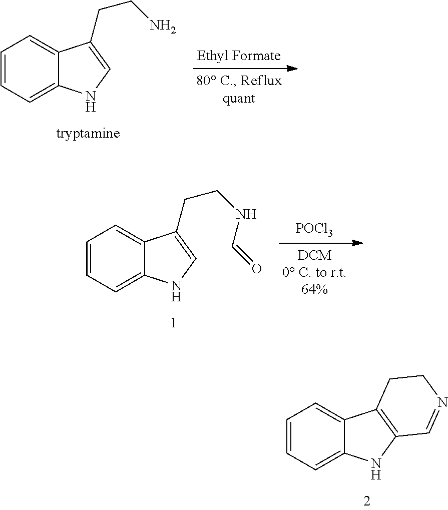

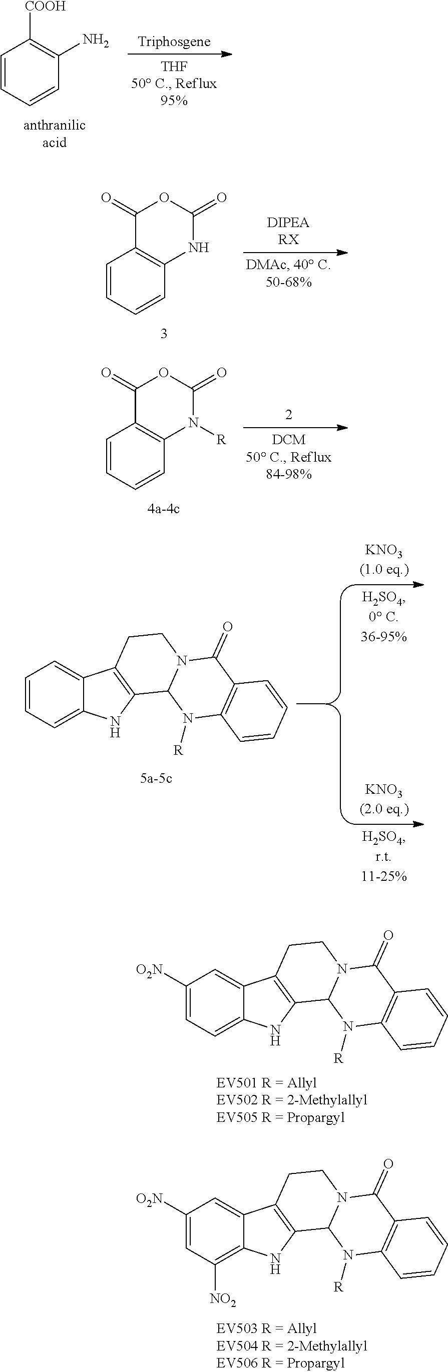

### N-formyltryptamine (1)

:Tryptamine (1.00 g, 6.24 mmol) and ethyl formate (2.3 g, 31.210 mmol) were heated under Ar substitution at 80° C. with stirring. After the reaction was complete, the excessive ethyl formate was removed by vacuum concentration, and the obtained mixture was purified by column chromatography (silica gel, MC:MeOH=20:1˜10:1) to yield compound 1 (1.2 g, 100% yield) in the form of a brown oil.

¹H-NMR (400 MHz, CDCl₃) δ 8.13 (br s, 2H), 7.61 (d, J=8.3 Hz, 1H), 7.38-7.40 (m, 1H), 7.23 (td, J=7.6, 1.4 Hz, 1H), 7.14 (t, J=7.6 Hz, 1H), 7.06 (s, 1H), 5.58 (br s, 1H), 3.64-3.69 (m, 2H), 3.01 (t, J=6.9 Hz, 2H); ¹³C-NMR (100 MHz, CDCl₃) b 161.5, 136.5, 127.3, 122.4, 122.2, 119.5, 118.7, 112.4, 111.5, 42.1, 38.4, 27.4, 25.2; HRMS (FAB) m/z calcd for C₁₁H₁₂N₂O [M+H]⁺: 188.0950, found 188.0955.

### 3,4-dihydro-β-carboline (2)

:N-formyltryptamine (1) (1.1 g, 5.920 mmol) was dissolved in CH₂Cl₂ (15.0 mL), after which POCl₃ (1.7 mL, 17.770 mmol) was added slowly at 0° C. The mixture was stirred for 2 hours at 0° C., then for 2 hours at room temperature. After the reaction was complete, excessive POCl₃ and CH₂Cl₂ were removed by vacuum concentration, and 1 M HCl (100 mL) was added to the obtained mixture, which was then washed once with CH₂Cl₂ (1×30 mL). The washed aqueous phase was adjusted to pH=10 with 1 M NaOH at 0° C., and then extracted three times with CH₂C₁₂ (3×30 mL). The obtained organic phase was dried and filtered with MgSO₄, and concentrated under vacuum. The obtained mixture was treated with Et₂O to precipitate solids, which were then filtered to yield compound 2 (4.0 g, 64% yield) as a yellow solid.

m.p.: 99-101° C.; ¹H-NMR (400 MHz, DMSO-d₆) δ 11.32 (s, 1H), 8.36 (t, J=2.3 Hz, 1H), 7.55 (d, J=8.2 Hz, 1H), 7.41 (d, J=8.2 Hz, 1H), 7.17-7.21 (m, 1H), 7.04 (td, J=7.5, 0.9 Hz, 1H), 3.78 (td, J=8.7, 2.1 Hz, 2H), 2.80 (t, J=8.7 Hz, 2H); ¹³C-NMR (100 MHz, DMSO-d₆) δ 151.6, 136.7, 128.4, 124.8, 123.7, 119.7, 119.6, 113.8, 112.5, 48.1, 18.7; HRMS (FAB) m/z calcd for C₁₁H₁₁N₂ [M+H]⁺: 171.0917, found 171.0921.

### Isatoic Anhydride (3)

:Anthranilic acid (1.1 g, 8.090 mmol) was dissolved in THF (15.0 mL) and then triphosgene (7.2 g, 24.280 mmol) was added at room temperature. The reaction temperature was then raised to 50° C., and the mixture was stirred for 3 hours. After the reaction was complete, ice water (50 mL) was added to the mixture, and the white solid compound 3 (1.3 g, 95% yield) was obtained by vacuum filtration.

m.p.: 164-165° C.; ¹H-NMR (400 MHz, DMSO-d₆) δ 11.73 (s, 1H), 7.91 (dd, J=7.8, 1.4 Hz, 1H), 7.72-7.76 (m, 1H), 7.23-7.27 (m, 1H), 7.15 (d, J=7.8 Hz, 1H); ¹³C-NMR (100 MHz, DMSO-d₆) δ 159.9, 147.1, 141.4, 137.00, 129.00, 123.5, 115.4, 110.3; HRMS (FAB) m/z calcd for C₈H₅NO₃ [M+H]⁺: 164.0269, found 164.0271.

### General Alkylation Conditions for Isatoic Anhydride

:Isatoic anhydride (3) (1.0 equiv.) was dissolved in DMAc (0.4 M), and then DIPEA (3.0 equiv.) and alkyl halide (3.0 equiv.) were added slowly, followed by stirring overnight at 40° C. Afterwards, H₂₀ was added to the mixture, and after the reaction was complete, the mixture was extracted several times with CH₂C₁₂. The collected organic phase was dried with MgSO₄, filtered, and concentrated under vacuum. The obtained mixture was treated with hexane to precipitate solids, which were then filtered to yield 4a-4c.

### N-Allyl Isatoic Anhydride (4a)

:Under the general alkylation conditions, isatoic anhydride (3) (200.0 mg, 1.226 mmol) and allyl bromide (445.0 mg, 3.678 mmol) were used to yield compound 4a (106.0 mg, 50% yield) as a beige solid.

m.p.: 127-128° C.; ¹H-NMR (400 MHz, DMSO-d₆) δ 8.02 (dd, J=8.0, 1.1 Hz, 1H), 7.79-7.84 (m, 1H), 7.31-7.35 (m, 2H), 5.87-5.96 (m, 1H), 5.31 (dq, J=17.5, 1.7 Hz, 1H), 5.20 (dq, J=10.6, 1.5 Hz, 1H), 4.67 (td, J=3.2, 1.5 Hz, 2H); ¹³C-NMR (100 MHz, DMSO-d₆) δ 159.0, 147.6, 141.3, 137.0, 131.2, 129.5, 123.6, 117.2, 115.2, 111.9, 46.5, 40.2, 39.9, 39.7, 39.5, 39.3, 39.1, 38.90; LRMS (FAB) m/z, 334 ([M+H]⁺).

### N-(2-methyl)allyl isatoic anhydride (4b)

:Under the general alkylation conditions, isatoic anhydride (3) (200.0 mg, 1.226 mmol) and 3-bromo-2-methylpropene (496.6 mg, 3.678 mmol) were used to obtain a beige solid compound 4b (151.6 mg, 57% yield).

m.p.: 124-125° C.; ¹H-NMR (400 MHz, DMSO-d₆) δ 8.02 (dd, J=7.7, 1.5 Hz, 1H), 7.78-7.82 (m, 1H), 7.31-7.35 (m, 1H), 7.24 (d, J=8.6 Hz, 1H), 4.82-4.87 (m, 2H), 4.55 (s, 2H), 1.80 (s, 3H); ¹³C-NMR (100 MHz, DMSO-d₆) δ 159.0, 147.8, 141.5, 138.2, 136.9, 129.4, 123.7, 115.4, 111.8, 111.0, 49.5, 19.8; LRMS (FAB) m/z, 334 ([M+H]⁺).

### N-Propargyl Isatoic Anhydride (4c)

:Under the general conditions B of N-alkylation, isatoic anhydride (6) (200.0 mg, 1.226 mmol) and propargyl bromide (437.5 mg, 3.678 mmol) were used to obtain a beige solid compound 4c (166.6 mg, 68% yield).

m.p.: 176-177° C.; ¹H-NMR (400 MHz, DMSO-d₆) δ 8.04 (d, J=7.8 Hz, 1n), 7.91 (t, J=7.8 Hz, 1H), 7.51 (d, J=8.7 Hz, 1H), 7.38 (t, J=7.6 Hz, 1H), 4.89 (s, 2H), 3.43 (s, 1H); ¹³C-NMR (100 MHz, DMSO-d₆) δ 158.5, 147.3, 140.5, 137.2, 129.6, 124.1, 115.0, 111.9, 77.6, 76.1, 34.4; LRMS (FAB) m/z, 202 ([M+H]⁺).

### General Condensation Reaction Conditions for Compound 5

:N-alkyl isatoic anhydride (4) (1.0 equiv.) and 3,4-dihydro-β-carboline (2) (1.1 equiv.) were dissolved in CH₂Cl₂ (0.4 M), then stirred at 50° C. under Ar substitution for 6 hours. The formed solid was filtered to obtain 5a-5c.

### 14N-Allyl Evodiamine (5a)

:Under the general condensation reaction conditions, 3,4-dihydro-3-carboline (2) (92.2 mg, 0.541 mmol) and N-allyl isatoic anhydride (4a) (100.0 mg, 0.492 mmol) were used to obtain a yellow solid compound 5a (125.1 mg, 84% yield).

m.p.: 204-206° C.; ¹H-NMR (400 MHz, DMSO-d₆) δ 11.08 (s, 1H), 7.79 (d, J=7.8 Hz, 1H), 7.42-7.47 (m, 2H), 7.35 (d, J=7.8 Hz, 1H), 7.11 (dd, J=7.6, 5.3 Hz, 2H), 6.97 (td, J=15.1, 7.8 Hz, 2H), 6.15 (s, 1H), 5.85 (dq, J=22.4, 5.4 Hz, 1H), 5.02-5.07 (m, 2H), 4.62 (dd, J=12.9, 5.1 Hz, 1H), 3.94 (d, 2H), 3.23 (td, J=12.2, 4.6 Hz, 1H), 2.90-2.98 (m, 1H), 2.76 (dd, J=15.6, 4.1 Hz, 1H). ¹³C-NMR (100 MHz, DMSO-d₆) b 164.5, 147.2, 136.4, 134.5, 133.1, 130.8, 128.0, 126.1, 121.8, 120.4, 120.0, 118.9, 118.5, 118.2, 117.1, 111.7, 111.4, 69.0, 52.6, 41.2, 19.3; HRMS (FAB) m/z calcd for C₂₁H₂ON₃O [M+H]⁺: 330.1606, found 330.1607.

### 14N-(2-methyl)allyl evodiamine (5b)

:Under the general condensation reaction conditions, 3,4-dihydro-3-carboline (2) (43.1 mg, 0.253 mmol) and N-(2-methyl)allyl isatoic anhydride (4b) (50.0 mg, 0.230 mmol) were used to obtain a dark yellow solid compound 5b (75.0 mg, 95% yield).

m.p.: 181-183° C.; ¹H-NMR (400 MHz, DMSO-d₆) δ 10.94 (s, 1H), 7.72 (dd, J=7.9, 1.8 Hz, 1H), 7.32-7.43 (m, 3H), 7.08 (t, J=7.9 Hz, 1H), 6.98 (t, J=7.9 Hz, 1H), 6.91 (d, J=7.9 Hz, 1H), 6.80 (t, J=7.0 Hz, 1H), 6.20 (s, 1H), 4.88 (d, J=21.4 Hz, 2H), 4.62 (dd, J=13.4, 5.5 Hz, 1H), 4.22 (d, J=16.5 Hz, 1H), 3.94 (d, J=16.5 Hz, 1H), 3.26 (td, J=12.2, 4.9 Hz, 1H), 2.95-3.03 (m, 1H), 2.68 (dd, J=15.6, 4.6 Hz, 1H), 1.72 (s, 3H); ¹³C-NMR (100 MHz, DMSO-d₆) δ 164.9, 146.6, 141.3, 136.1, 133.3, 132.1, 128.0, 126.3, 121.7, 118.9, 118.8, 118.1, 117.4, 115.8, 112.3, 111.7, 111.2, 70.1, 54.9, 42.2, 20.0, 19.2; HRMS (FAB) m/z calcd for C₂₂H₂₂N₃O [M+H]⁺: 344.1763, found 344.1759.

### 14N-propagyl Evodiamine (5c)

:Under the general condensation reaction conditions, 3,4-dihydro-3-carboline (2) (93.1 mg, 0.547 mmol) and N-propargyl isatoic anhydride (4c) (100.0 mg, 0.497 mmol) were used to obtain a yellow solid compound 5c (159.2 mg, 98% yield).

m.p.: 182-184° C.; ¹H-NMR (400 MHz, DMSO-d₆) δ 11.26 (s, 1H), 7.90 (dd, J=7.8, 1.4 Hz, 1H), 7.50-7.56 (m, 2H), 7.38 (d, J=8.3 Hz, 1H), 7.31 (d, J=7.4 Hz, 1H), 7.12-7.19 (m, 2H), 7.02-7.06 (m, 1H), 6.16 (s, 1H), 4.62 (ddd, J=12.8, 4.9, 2.0 Hz, 1H), 4.04 (dd, J=18.2, 2.5 Hz, 1H), 3.61 (dd, J=17.9, 2.3 Hz, 1H), 3.18-3.25 (m, 1H), 3.04 (t, J=2.3 Hz, 1H), 2.84-2.98 (m, 2H); ¹³C-NMR (100 MHz, DMSO-d₆) δ 163.5, 147.1, 136.8, 132.8, 129.0, 128.0, 125.8, 123.4, 122.9, 122.1, 121.3, 119.0, 118.5, 112.2, 111.7, 79.9, 75.2, 68.1, 19.5; HRMS (FAB) m/z calcd for C₂₁H₁₈N₃O [M+H]⁺: 328.1450, found 328.1456.

### General Nitration Conditions for Evodiamine Derivatives

:KNO₃ (1.0 equiv.) and concentrated H₂SO₄ (0.33 M) were added to 14N-alkyl evodiamine (5) (1.0 equiv.), then stirred overnight at 0° C. After the reaction ended, ice water was added to the mixture, and the resulting solid was vacuum filtered to obtain EV501-503.

### EV501

:Under the general nitration conditions, 5a (200.0 mg, 0.607 mmol) and KNO₃ (61.4 mg, 0.607 mmol) were used to obtain a yellow solid compound EV501 (83.8 mg, 37% yield).

m.p.: 186-188° C.; ¹H-NMR (400 MHz, DMSO-d₆) δ 11.92 (s, 1H), 8.49 (d, J=2.3 Hz, 1H), 8.01 (dd, J=9.2, 2.3 Hz, 1H), 7.78 (dd, J=7.8, 1.4 Hz, 1H), 7.43-7.51 (m, 2H), 7.12 (d, J=8.3 Hz, 1H), 6.95 (t, J=7.7 Hz, 1H), 6.22 (s, 1H), 5.87 (ddd, J=23.0, 10.1, 6.0 Hz, 1H), 5.04-5.10 (m, 2H), 4.63 (dd, J=12.9, 4.6 Hz, 1H), 3.98 (d, J=4.1 Hz, 2H), 3.26 (dd, J=13.1, 4.8 Hz, 1H), 2.96-3.04 (m, 1H), 2.88 (dd, J=15.6, 4.6 Hz, 1H); ¹³C-NMR (100 MHz, DMSO-d₆) δ 164.1, 147.9, 139.5, 136.2, 134.7, 133.0, 131.9, 131.1, 129.8, 128.1, 122.0, 121.6, 121.5, 120.1, 117.3, 116.9, 114.4, 67.6, 52.8, 19.4; HRMS (FAB) m/z calcd for C₂₁H₁₉N₄O₃ [M+H]⁺: 375.1457, found 375.1459.

### EV502

:Under the general nitration conditions, 5b (87.9 mg, 0.226 mmol) and KNO₃ (22.9 mg, 0.226 mmol) were used to obtain a yellow solid compound EV502 (31.6 mg, 36% yield).

m.p.: 261-262° C.; ¹H-NMR (400 MHz, DMSO-d₆) δ 11.94 (s, 1H), 8.52 (d, J=2.3 Hz, 1H), 8.04 (dd, J=9.2, 2.3 Hz, 1H), 7.71 (dd, J=7.8, 1.4 Hz, 1H), 7.55 (m, 2H), 7.49-7.44 (m, 1H), 7.01 (t, J=7.7 Hz, 1H), 6.95 (d, J=4.1 Hz, 1H), 6.11 (s, 1H), 4.98 (d, J=4.1 Hz, 2H), 4.64 (dd, J=13.8, 4.3 Hz, 1H), 4.06 (d, J=4.6 Hz, 1H), 3.78 (d, J=4.3 Hz, 1H), 3.21 (dt, J=13.1, 4.8 Hz, 1H), 3.07-2.99 (m, 1H), 2.81 (dd, J=15.8, 4.1 Hz, 1H), 1.86 (s, 3H); ¹³C-NMR (100 MHz, DMSO-d₆) δ 165.2, 147.4, 140.5, 139.2, 133.6, 131.9, 130.2, 127.7, 121.6, 119.2, 118.5, 117.2, 116.4, 115.4, 115.1, 114.0, 113.5, 72.6, 59.1, 20.4, 18.44; LRMS (FAB) m/z, 389 ([M+H]⁺).

### EV505

:Under the general nitration conditions, 5c (100.0 mg, 0.305 mmol) and KNO₃ (30.9 mg, 0.305 mmol) were used to obtain a yellow solid compound EV505 (107.6 mg, 94.8% yield).

m.p.: 164-166° C.; ¹H-NMR (400 MHz, DMSO-d₆) δ 12.10 (s, 1H), 8.57 (d, J=2.0 Hz, 1H), 8.05 (dd, J=8.9, 2.2 Hz, 1H), 7.89 (dd, J=7.7, 1.3 Hz, 1H), 7.52-7.58 (m, 2H), 7.32 (d, J=8.1 Hz, 1H), 7.16-7.20 (m, 1H), 6.24 (s, 1H), 4.60-4.66 (m, 1H), 4.08 (dd, J=18.1, 2.4 Hz, 1H), 3.69 (dd, J=18.1, 2.4 Hz, 1H), 3.22-3.29 (m, 1H), 3.08 (t, J=2.4 Hz, 1H), 3.00 (d, J=4.7 Hz, 2H); ¹³C-NMR (100 MHz, DMSO-d₆) δ 163.5, 146.9, 140.7, 139.9, 133.5, 133.1, 128.0, 125.3, 123.1, 121.4, 117.5, 115.9, 114.8, 112.2, 79.8, 75.5, 68.0, 19.3; HRMS (FAB) m/z calcd for C₂₁H₁₇N₄O₃ [M+H]⁺: 373.1301, found 373.1305.

### General Dinitration Conditions for Evodiamine Derivatives

:KNO₃ (2.0 equiv.) and concentrated H₂SO₄ (0.33 M) were added to 14N-alkyl evodiamine (5) (1.0 equiv.) at 0° C., then stirred overnight at room temperature. After the reaction ended, ice water was added to the mixture, and the resulting solid was purified by column chromatography (silica gel, hexane:ethyl acetate=2:1-1:1) to obtain EV504-506.

### EV503

:Under the general dinitration conditions, 5a (200.0 mg, 0.607 mmol) and KNO₃ (122.8 mg, 1.214 mmol) were used to obtain a yellow solid compound EV503 (28.8 mg, 11.3% yield).

m.p.: 229-231° C.; ¹H-NMR (400 MHz, DMSO-d₆) δ 12.51 (s, 1H), 9.02 (d, J=2.0 Hz, 1H), 8.82 (d, J=2.0 Hz, 1H), 7.86 (dd, J=7.9, 1.5 Hz, 1H), 7.48-7.52 (m, 1H), 7.26 (d, J=8.4 Hz, 1H), 7.07 (t, J=7.4 Hz, 1H), 6.16 (s, 1H), 5.68-5.78 (m, 1H), 4.86-4.91 (m, 2H), 4.66 (dd, J=12.3, 4.9 Hz, 1H), 3.95 (dd, J=16.3, 5.9 Hz, 1H), 3.63 (dd, J=16.3, 6.2 Hz, 1H), 3.12-3.23 (m, 2H), 2.89-2.97 (m, 1H); ¹³C-NMR (100 MHz, DMSO-d₆) δ 164.1, 147.9, 139.5, 136.2, 134.7, 133.0, 131.9, 131.1, 129.9, 128.1, 122.0, 121.6, 121.4, 120.1, 117.3, 116.9, 114.4, 67.6, 52.8, 19.4; HRMS (FAB) m/z calcd for C₂₁H₁₈N₅O₈ [M+H]⁺: 420.1308, found 420.1301.

### EV504

:Under the general dinitration conditions, 5b (211.0 mg, 0.614 mmol) and KNO₃ (124.2 mg, 1.229 mmol) were used to obtain a yellow solid compound EV504 (34.6 mg, 13% yield).

m.p.: 175-177° C.; ¹H-NMR (400 MHz, DMSO-d₆) δ 12.55 (s, 1H), 9.04 (d, J=2.0 Hz, 1H), 8.84 (d, J=2.0 Hz, 1H), 7.91 (dd, J=7.7, 1.7 Hz, 1H), 7.50-7.54 (m, 1H), 7.15 (t, J=7.6 Hz, 1H), 7.07 (d, J=7.7 Hz, 1H), 6.17 (s, 1H), 5.29 (dd, J=30.7, 14.3 Hz, 2H), 5.07 (d, J=16.8 Hz, 2H), 4.66-4.71 (m, 1H), 3.90 (d, J=16.5 Hz, 1H), 3.63 (d, J=16.5 Hz, 1H), 3.34-3.41 (m, 1H), 3.15-3.21 (m, 1H), 2.89-2.96 (m, 1H), 1.99 (s, 1H); ¹³C-NMR (100 MHz, DMSO-d₆) δ 165.3, 147.7, 140.6, 139.5, 133.7, 132.0, 130.3, 127.9, 121.8, 119.3, 118.7, 117.4, 116.7, 115.6, 115.3, 114.2, 113.7, 72.9, 59.8, 20.8, 18.7; LRMS (FAB) m/z, 434 ([M+H]⁺).

### EV506

:Under the general dinitration conditions, 5c (166.2 mg, 0.508 mmol) and KNO₃ (102.6 mg, 1.015 mmol) were used to obtain a yellow solid compound EV506 (48.3 mg, 25% yield).

m.p.: 232-234° C.; ¹H-NMR (400 MHz, DMSO-d₆) δ 12.53 (s, 1H), 12.11 (s, 1H), 9.07 (d, J=2.0 Hz, 1H), 8.84 (d, J=2.4 Hz, 1H), 7.87-7.92 (m, 3H), 7.52-7.59 (m, 4H), 7.37 (d, J=8.1 Hz, 1H), 7.14-7.21 (m, 3H), 6.27-6.23 (1H), 4.64-4.71 (m, 1H), 4.12 (dd, J=14.3, 2.5 Hz, 1H), 3.60 (dd, J=18.5, 2.4 Hz, 1H), 3.22-3.29 (m, 1H), 3.12 (t, J=2.2 Hz, 1H), 3.00 (d, J=7.4 Hz, 2H); ¹³C-NMR (100 MHz, DMSO-d₆) δ 163.3, 147.1, 139.5, 135.7, 131.9, 131.4, 129.8, 128.0, 123.1, 122.1, 121.3, 117.6, 114.5, 112.1, 80.1, 75.6, 67.5, 40.1, 39.9, 39.7, 39.5, 39.3, 39.1, 38.9, 19.4; HRMS (FAB) m/z calcd for C₂₁H₁₆N₅O₅ [M+H]⁺: 418.1151, found 418.1158.

### 6-2. Method for Synthesis of EV507-509

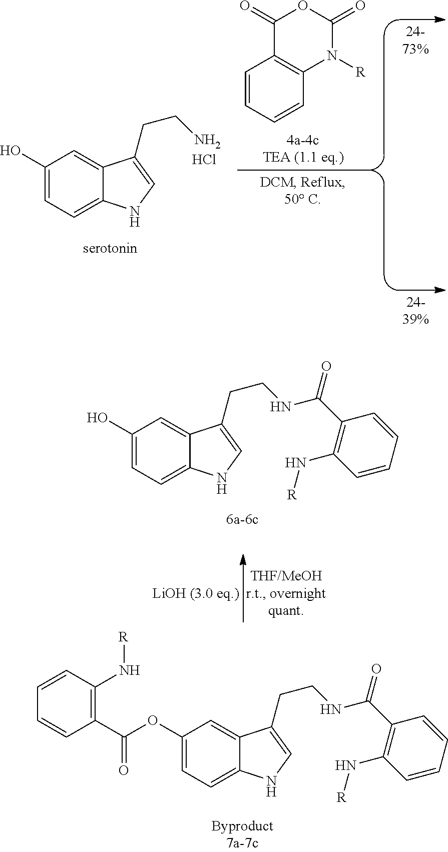

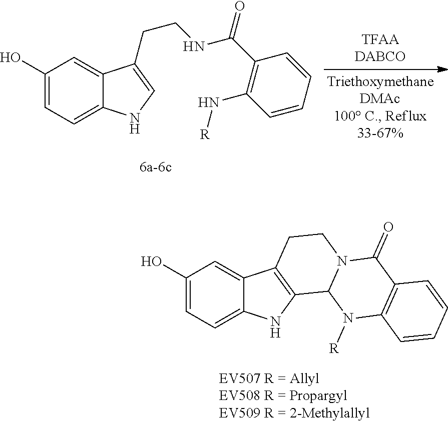

### General Bond Cleavage Reaction Conditions for Compound 6

:N-alkyl isatoic anhydride (4) (1.0 equiv.) and serotonin hydrochloride (1.1 equiv.) were dissolved in CH₂C₁₂ (0.4 M), followed by the addition of triethylamine (1.0 equiv.). The temperature was then raised to 50° C. and refluxed with stirring for 3 hours. After the completion of the reaction, excess CH₂Cl₂ was removed by vacuum concentration. The resulting mixture was purified by column chromatography (silica gel, hexane:ethyl acetate=2:1˜1:1) to obtain the desired intermediate 6 and by-product 7.

Following the general bond cleavage reaction conditions, serotonin hydrochloride (300.0 mg, 1.411 mmol) and N-allyl isatoic anhydride (4a) (286.6 mg, 1.411 mmol) were used to obtain a colorless oil compound 6a (135.0 mg, 29% yield) and a white solid compound 7a (198.4 mg, 28% yield).

### 2-(allylamino)-N-(2-(5-hydroxy-1H-indol-3-yl)ethyl)benzamide (6a)

¹H-NMR (400 MHz, CDCl₃) δ 7.91 (s, 1H), 7.70 (s, 1H), 7.14-7.16 (m, 1H), 7.04 (dd, J=8.1, 2.0 Hz, 2H), 6.78 (dd, J=8.6, 2.9 Hz, 1H), 6.67 (d, J=8.7 Hz, 1H), 6.52 (t, J=7.1 Hz, 1H), 6.13 (s, 1H), 5.91-6.00 (m, 1H), 5.28-5.33 (m, 1H), 5.17 (dd, J=10.1, 1.3 Hz, 1H), 4.74 (s, 1H), 3.84 (d, J=4.7 Hz, 2H), 3.70 (q, J=6.5 Hz, 2H), 3.02 (t, J=6.7 Hz, 2H); ¹³C-NMR (100 MHz, CDCl₃) δ 171.4, 170.3, 149.8, 149.3, 132.7, 132.2, 127.9, 127.4, 123.3, 116.3, 115.1, 112.6, 112.2, 112.1, 111.5, 103.5, 77.5, 77.2, 76.8, 46.0, 45.7, 40.2, 25.4; LRMS (FAB) m/z, 336 ([M+H]⁺).

### 3-(2-(2-(allylamino)benzamido)ethyl)-1H-indol-5-yl2-(allylamino)benzoate (7a)

m.p.: 67-69° C.; ¹H-NMR (400 MHz, CDCl₃) δ 8.19 (dd, J=8.1, 1.3 Hz, 1H), 8.17 (s, 1H), 7.89 (t, J=5.0 Hz, 1H), 7.73 (s, 1H), 7.36-7.45 (m, 3H), 7.23 (td, J=7.9, 1.6 Hz, 1H), 7.16-7.17 (m, 1H), 7.06 (d, J=2.0 Hz, 1H), 7.01 (dd, J=8.6, 1.8 Hz, 1H), 6.62-6.73 (m, 3H), 6.52 (t, J=8.1 Hz, 1H), 6.13 (t, J=5.4 Hz, 1H), 5.88-5.98 (m, 2H), 5.26-5.32 (m, 2H), 5.13-5.18 (m, 2H), 3.85-3.90 (m, 2H), 3.80 (d, J=5.4 Hz, 2H), 3.72 (q, J=6.5 Hz, 2H), 3.04 (t, J=6.7 Hz, 2H); ¹³C-NMR (100 MHz, CDCl₃) δ 170.0, 168.5, 151.7, 149.4, 144.3, 135.3, 135.0, 134.4, 132.7, 132.3, 127.8, 127.4, 123.7, 116.9, 116.4, 116.2, 115.6, 115.1, 115.0, 113.4, 112.1, 111.9, 111.8, 111.3, 109.4, 45.6, 45.4, 39.9, 25.4; LRMS (FAB) m/z, 495 ([M+H]⁺).

### General Hydrolysis Conditions for by-Product 7

:By-product 7 (1.0 equiv.) was dissolved in THF (0.06 M), and LiOH (3.0 equiv.) was dissolved in MeOH (0.06 M) and slowly added to the reaction mixture. After stirring at room temperature for 12 hours, the mixture was concentrated under vacuum to remove excess THF and MeOH. The obtained mixture was dissolved in ethyl acetate (30 mL) and the organic phase was washed with water three times (10 mL×3). The aqueous phase was then extracted three times with ethyl acetate (10 mL×3). The obtained organic phase was dried and filtered with MgSO₄, and then concentrated under vacuum. The obtained mixture was purified by column chromatography (silica gel, hexane:ethyl acetate=2:1˜1:1) to quantitatively obtain the desired intermediate 6.

### General Cyclization Reaction Conditions for Intermediate 6

:Intermediate 6 (1.0 equiv.) was dissolved in triehoxymethane-DMF (0.33 M), and trifluoroacetic anhydride (1.0 equiv.) and DABCO (1.1 equiv.) were sequentially added. The reaction temperature was then increased to 100° C. and refluxed with stirring for 6 hours. After completion of the reaction, the mixture was dissolved in ethyl acetate, and the organic phase was washed with water three times. The aqueous phase was then extracted three times with ethyl acetate. The obtained organic phase was dried and filtered with MgSO₄, and then concentrated under vacuum. The obtained mixture was purified by column chromatography (silica gel, hexane:ethyl acetate=2:1˜1:1) to obtain EV507-509.

### EV507

:Under the general cyclization reaction conditions, compound 6a (135.0 mg, 0.402 mmol) was used to obtain a yellow solid compound EV507 (50.4 mg, 36% yield).

m.p.: 234-236° C.; ¹H-NMR (400 MHz, DMSO-d₆) δ 10.74 (s, 1H), 8.70 (s, 1H), 7.77 (dd, J=8.1, 1.3 Hz, 1H), 7.41-7.45 (m, 1H), 7.13 (d, J=8.8 Hz, 1H), 7.07 (d, J=8.8 Hz, 1H), 6.92 (t, J=7.1 Hz, 1H), 6.74 (d, J=2.0 Hz, 1H), 6.61 (dd, J=8.4, 2.4 Hz, 1H), 6.10 (s, 1H), 5.80-5.89 (m, 1H), 5.03-5.09 (m, 2H), 4.59 (dd, J=13.1, 5.1 Hz, 1H), 3.94 (d, J=4.7 Hz, 2H), 3.16-3.24 (m, 1H), 2.82-2.90 (m, 1H), 2.64 (dd, J=15.2, 4.4 Hz, 1H); ¹³C-NMR (100 MHz, DMSO-d₆) δ 164.6, 150.7, 147.1, 134.5, 133.1, 131.4, 130.8, 128.0, 126.9, 120.2, 119.8, 118.2, 117.1, 112.1, 110.5, 102.1, 69.2, 52.5, 41.3, 19.4; HRMS (FAB) m/z calcd for C₂₁H₂₀N₃O₂ [M+H]⁺: 346.1556, found 346.1558.

### EV508

:Under the general cyclization reaction conditions, compound 6c (81.1 mg, 0.243 mmol) was used to obtain a yellow solid compound EV508 (56.2 mg, 67% yield).

m.p.: 230-232° C.; ¹H-NMR (400 MHz, DMSO-d₆) δ 10.91 (s, 1H), 8.75 (s, 1H), 7.88 (dd, J=8.1, 1.3 Hz, 1H), 7.51-7.55 (m, 1H), 7.29 (d, J=7.4 Hz, 1H), 7.15 (dd, J=8.1, 6.0 Hz, 2H), 6.80 (d, J=2.0 Hz, 1H), 6.65 (dd, J=8.7, 2.0 Hz, 1H), 6.11 (s, 1H), 4.59 (td, J=6.3, 4.8 Hz, 1H), 4.00-4.05 (m, 2H), 3.62 (dd, J=18.1, 2.0 Hz, 1H), 3.19 (td, J=12.1, 4.1 Hz, 1H), 3.05 (t, J=2.4 Hz, 1H), 2.83-2.90 (m, 1H), 2.72-2.76 (m, 1H); ¹³C-NMR (100 MHz, DMSO-d₆) δ 163.5, 150.8, 147.1, 132.8, 131.2, 129.4, 128.0, 126.6, 123.3, 122.8, 121.2, 112.3, 112.1, 111.3, 102.3, 80.0, 75.2, 68.2, 40.2, 39.9, 39.7, 39.5, 39.3, 39.1, 39.0, 38.9, 19.6; HRMS (FAB) m/z calcd for C₂₁H₁₈N₃O₂ [M+H]⁺:344.1399, found 344.1407.

### EV509

:Under the general cyclization reaction conditions, compound 6b (95.8 mg, 0.274 mmol) was used to obtain a yellow solid compound EV509 (32.7 mg, 33% yield).

m.p.: 211-213° C.; ¹H-NMR (400 MHz, DMSO-d₆) δ 10.59 (s, 1H), 8.67 (s, 1H), 7.71 (dd, J=7.8, 1.7 Hz, 1H), 7.34-7.38 (m, 1H), 7.11 (d, J=8.8 Hz, 1H), 6.89 (d, J=8.1 Hz, 1H), 6.79 (t, J=7.1 Hz, 1H), 6.70 (d, J=2.0 Hz, 1H), 6.59 (dd, J=8.4, 2.4 Hz, 1H), 6.14 (s, 1H), 4.89 (d, J=22.2 Hz, 2H), 4.59 (q, J=6.3 Hz, 1H), 4.21 (d, J=16.2 Hz, 1H), 3.92 (d, J=16.2 Hz, 1H), 3.23 (td, J=12.1, 4.7 Hz, 1H), 2.87-2.95 (m, 1H), 2.57 (dd, J=15.5, 4.7 Hz, 1H), 1.72 (s, 3H); ¹³C-NMR (100 MHz, DMSO-d₆) b 163.5, 150.8, 147.1, 132.8, 131.2, 129.4, 128.0, 126.6, 123.2, 122.8, 121.2, 112.3, 112.1, 111.3, 102.3, 80.0, 75.2, 68.2, 19.6; LRMS (FAB) m/z, 360 ([M+H]⁺).

### Example 1. Experimental Method

### 1-1. Cell Culture

Human lung cancer cell lines (H₁₂₉₉, H₄₆₀, A549, H₂₂₆B, and PC9), breast cancer cell line (MDA-MB-231), liver cancer cell line (Hep3B), kidney cancer cell line (786-0), prostate cancer cell lines (DU145 and PC-3), and mouse lung cancer cell line (Lewis Lung Carcinoma, LLC) were purchased from ATCC (American Type Culture Collection) or obtained from Dr. John V. Heymach of MD Anderson Cancer Center, USA. The human head and neck cancer cell line (UMSCC38) was obtained from Dr. Jeffrey N. Myers (MD Anderson Cancer Center, USA). Human colon cancer cell lines (HCT116 and HT-29) and glioblastoma cell line (U87MG) were obtained from Professor Sang Kook Lee (Seoul National University College of Pharmacy). The human kidney cancer cell line (Caki-1), and gastric cancer cell line (SNU-638) were purchased from the Korean Cell Line Bank. The chemotherapy-resistant cells [paclitaxel-resistant H₄₆₀ cells (H₄₆₀/PcR), paclitaxel-resistant H₂₂₆B cells (H₂₂₆B/PcR), cisplatin-resistant H₁₂₉₉ cells (H₁₂₉₉/CsR), pemetrexed-resistant H₁₂₉₉ cells (H₁₂₉₉/PmR)] and molecularly targeted anticancer therapy-resistant cells [erlotinib-resistant PC9 cells (PC9/ER)] were constructed by treating the respective anti-cancer drugs for more than three months. H₁₂₉₉, H₄₆₀, H₂₂₆B, PC9, Hep3B, 786-0, DU145, PC-3, HCT116, HT-29, SNU-638, chemotherapeutic resistant cells, and molecularly targeted anticancer therapy-resistant cells were cultured in RPMI 1640 media containing 10% fetal bovine serum (FBS) and antibiotics at 37° C. under 5% CO₂ conditions, while MDA-MB-231, UMSCC38, U87MG, Caki-1 were cultured in DMEM media containing 10% FBS and antibiotics at 37° C. under 5% CO₂ conditions. Cells were passaged 1-2 times per week.

### 1-2. Cell Viability Assay

The cancer cell lines were seeded in a 96-well plate at a density of 2×10³ cells per well and cultured for 24 hours to allow the cells to attach. The cells were then treated with diluted drugs in media for 3 days and cell viability was assessed using MTT assay or crystal violet (CB) assay. For the MTT assay, MTT solution dissolved in PBS was added to achieve a final concentration of 500 μg/mL and incubated for 2-4 hours, after which the medium was removed and the formed formazan was dissolved in 100% DMSO, followed by measuring the absorbance at 570 nm. For the CB assay, the cultured cells were fixed with 100% methanol, stained with a 0.025% crystal violet solution, dissolved in 1% SDS solution, and the absorbance was measured at 570 nm. Cell viability was calculated as the percentage of absorbance change in drug-treated group compared to vehicle (DMSO) treated control group.

### 1-3. Evaluation of Anchorage-Dependent Colony Formation

Cancer cell lines were seeded in a 24-well or 6-well plate at 100-250 cells per well and allowed to attach, followed by treatment with drugs diluted in media and cultured for 2-3 weeks. Formed colonies were stained with crystal violet and counted. The drug's colony formation inhibitory effect was calculated as the percentage of colony number change in the drug-treated group compared to the vehicle (DMSO)-treated control group.

### 1-4. Evaluation of Anchorage-Independent (Soft Agar) Colony Formation

Before the experiment, a 1% agar solution made with low-melting agar was placed in a 24-well plate and hardened at room temperature to create a bottom agar. The cells were diluted with medium to seed 2×10³ cells per well in a 24-well plate. Then, it was mixed with a 1% agar solution to create a final 0.4% agar solution, which was then solidified at room temperature in the plate containing the bottom agar. Medium containing the drug was added on top of the cells mixed in the agar and cultured for 1-3 weeks at 37° C. in a 5% CO₂ condition, after which the formed colonies were stained with an MTT solution of 250-500 μg/mL and counted. The colony formation inhibitory effect of the drug was calculated by expressing the change in colony numbers in the drug-treated group compared to the vehicle (DMSO) treated control group as a percentage.

### 1-5. Western Blot Analysis

The cells treated with the drug were treated with RIPA lysis buffer [50 mM Tris-HCl (pH 7.4), 150 mM NaCl, 1 mM EDTA, 0.25% sodium deoxycholate, 1% Triton X-100, 100 mM NaF, 5 mM Na₃VO₄, 1 mM PMSF, 1 μg/mL aprotinin, 1 μg/mL leupeptin, and 1 μg/mL pepstatin], followed by incubation on ice for 10 minutes and centrifugation at 13,000 rpm for 15 minutes to extract proteins. The proteins were quantified by BCA assay and equal amounts of proteins were separated by 8-10% SDS-PAGE. The separated proteins were transferred to a PVDF membrane, and the areas of the membrane not transferred with protein were blocked by incubating the membrane in 5% skim milk solution or 3% BSA solution diluted in TBST at room temperature for 1 hour. The membrane was then incubated in a primary antibody solution diluted 1:1,000 in 3% BSA at 4° C. for more than 12 hours. The primary antibody used was either anti-Hsp70 or anti-actin, with the anti-Hsp70 antibody obtained from Enzo Life Science and the actin antibody obtained from Santa Cruz Biotechnology. After incubation, the membrane was washed for 1 hour while changing TBST several times, and then incubated for 1 hour at room temperature with a secondary antibody (GeneTex) diluted 1:5,000 in 5% skim milk solution. After incubation, the membrane was washed for 1 hour while changing TBST several times, and then treated with an enhanced chemiluminescence (ECL) solution. The expression of Hsp70 or Actin protein was detected using LAS4000.

### 1-6. Drug Affinity Responsive Target Stability (DARTS)

35 μg of Hsp70 full length, Hsp70 N-terminal, Hsp70 C-terminal proteins were treated with vehicle (DMSO) or up to 10 μM of EV507 (final DMSO concentration: 1%) at 4° C. for 30 minutes, followed by a treatment with proteinase K (proteinase K:protein=1:100) at room temperature for 15 minutes. The reaction was terminated by adding a 5×SDS-PAGE sample buffer and boiling at 95° C. for 5 minutes, after which electrophoresis was performed with 8% SDS-PAGE. The proteins were transferred to a PVDF membrane, and the Western blot analysis method described above was performed.

### 1-7. Animal Experiment

H₄₆₀ cells (5×10⁶ cells/spot) or lung cancer patient-derived tumors were implanted into the flank of 6-week-old NOD/SCID mice, or LLC-Luc cells (5×10⁵ cells/spot) were implanted into the flank of 8-week-old C57BL/6 mice to form tumors. When the tumor volume reached 50-100 mm³, the mice were divided into control and experimental groups (with at least 6 mice assigned to each group), and the control group was administered with vehicle (10% DMSO and 40% PEG400 solution prepared in sterile distilled water), while the experimental group was administered with EV507 (20 mg/kg) six times a week orally. The major and minor axes of the tumor were measured, and the tumor size was measured at least 3-4 times a week, and the body weight was measured at least once a week to evaluate the toxicity of the drug. Also, the survival of C57BL/6 mice implanted with LLC-Luc cells was monitored to determine the survival curve. The tumor volume was calculated as (width)²× length×0.5.

### 1-8. Statistical Analysis

All results were presented as mean±standard deviation, and statistical analysis was performed using two-tailed Student's t-test or Mann-Whitney test when there were two groups, and one-way ANOVA when there were three or more groups. Survival analysis was evaluated for significance using the log-rank test. Statistical significance analysis was conducted using Graphpad Prism (version 9). P value of less than 0.05 was considered statistically significant.

### Example 2. Confirmation of the Inhibition Effect on Cancer Cell Survival Rate and Colony Formation by Evodiamine Derivative EV507

The anti-cancer survival inhibition effect of EV compounds (EV501-EV509) was evaluated at a concentration of 100 nM in H₁₂₉₉ cells.

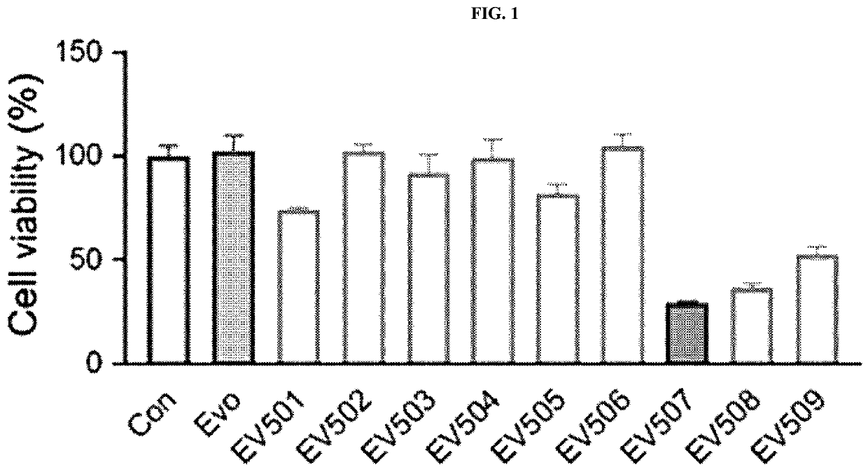

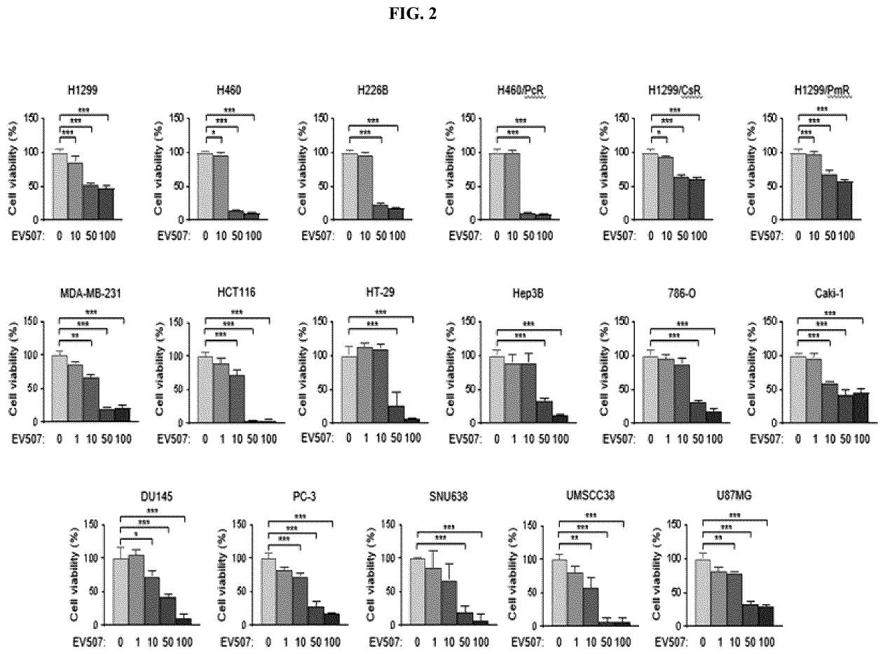

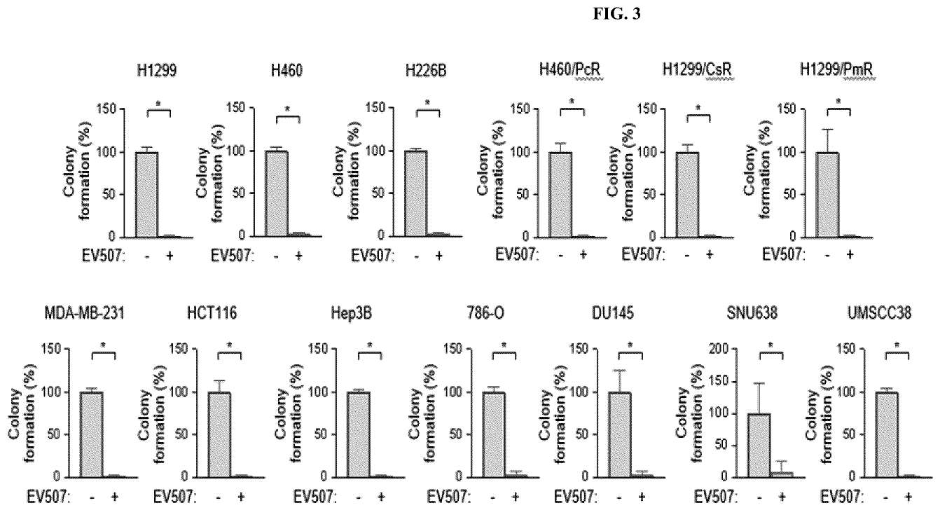

As shown in FIG. 1, the compound showing the most excellent efficacy, EV507, was discovered, and EV507 was found to have a significantly superior effect in inhibiting cancer cell survival than evodiamine and other evodiamine derivatives. Also, as shown in FIG. 2 and FIG. 3, EV507 demonstrated concentration-dependent anti-cancer survival inhibition efficacy across cancer cells derived from various origins and significantly inhibited the formation of colonies under anchorage-dependent conditions at a low concentration of 50 nM.

### Example 3. Confirmation of the Inhibition Effect of EV507 on Hsp70 and Client Protein Expression

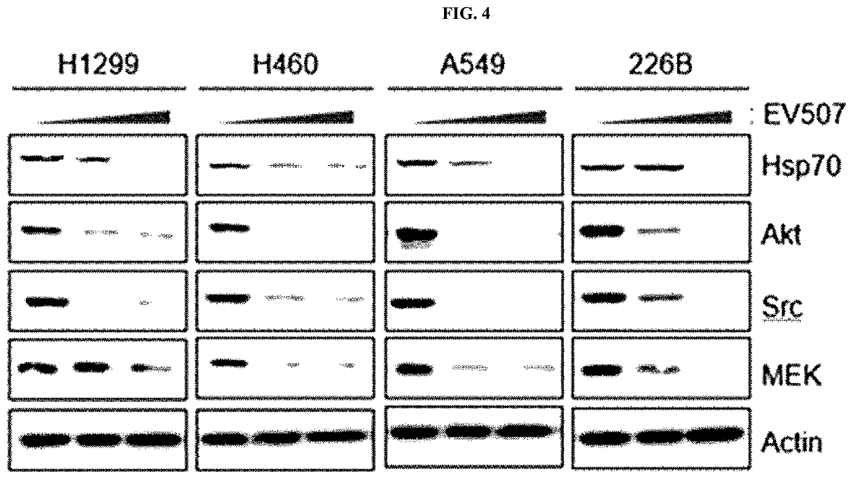

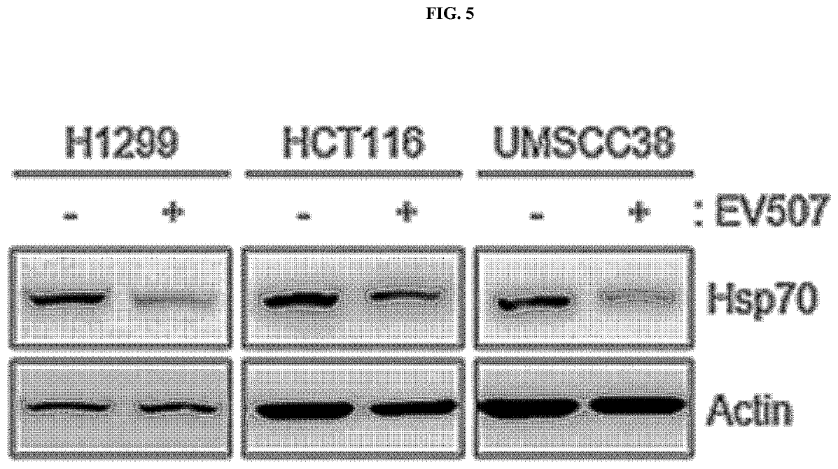

The effect of EV507 on Hsp70 and related client proteins was confirmed. As shown in FIG. 4 and FIG. 5, when various lung cancer cells were treated with EV507, a clear reduction was observed in the expression of Hsp70 protein and Hsp70/Hsp90 client proteins (Akt, Src, and MEK). The same effect was also observed in colon cancer cell line HCT116 and head and neck cancer cell line UMSCC38.

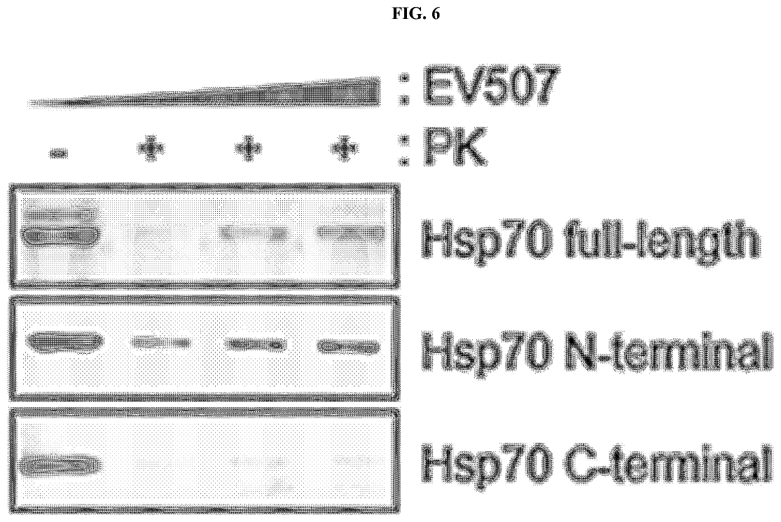

Furthermore, as shown in FIG. 6, it was confirmed that EV507 binds to the Hsp70 protein, particularly the Hsp70 N-terminal domain, through DARTS.

### Example 4. Anti-Cancer Effect of EV507

The anti-tumor effect of EV507 was confirmed in a mouse model transplanted with H₄₆₀ and tumors derived from lung cancer patients.

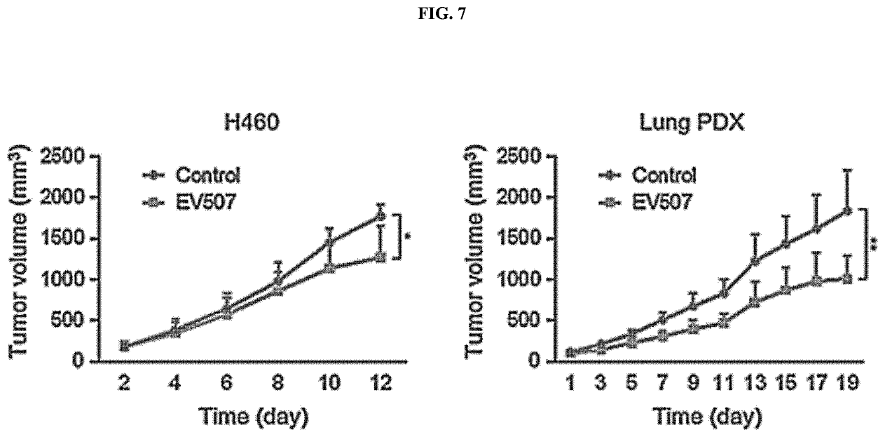

As shown in FIG. 7, EV507 significantly inhibited tumor growth compared to the control group.

Moreover, it is known that in the LLC-Luc transplant allograft model that spontaneously generates pulmonary metastatic cancer, mouse mortality occurs due to the proliferation of transplanted LLC-Luc tumors and the formation of pulmonary metastatic cancer. In this allograft model, EV507 was administered to confirm its inhibitory effect on mouse mortality caused by tumor proliferation.

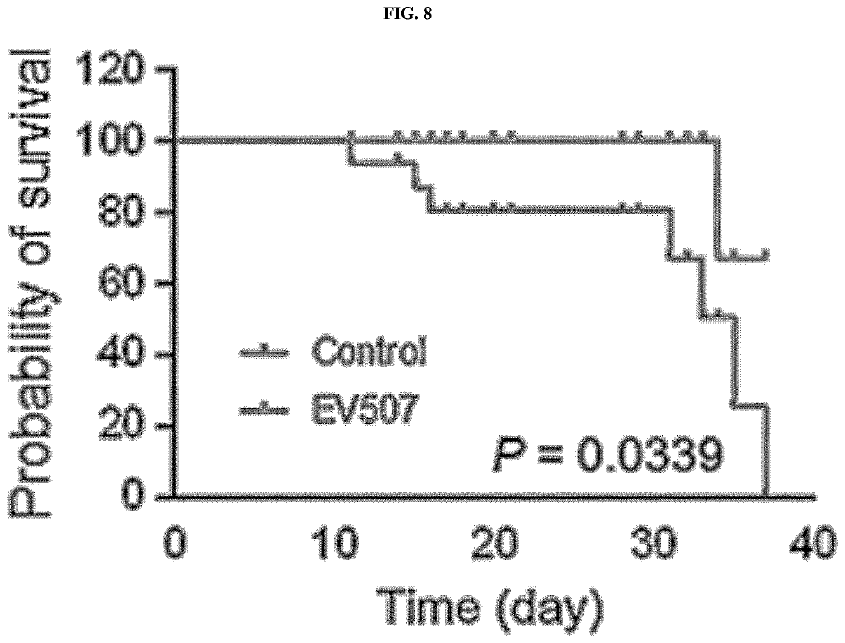

As shown in FIG. 8, it was confirmed that mouse mortality was significantly reduced by EV507 treatment, verifying the anti-cancer and cancer malignancy inhibiting action of EV507.

### Example 5. Confirmation of the Therapeutic Effect on Lung Cancer by Various Evodiamine Derivatives

Based on the anti-cancer effect of EV507 confirmed in the previous examples, the effect of various evodiamine derivatives on the death of H₁₂₉₉ lung cancer cells was confirmed.

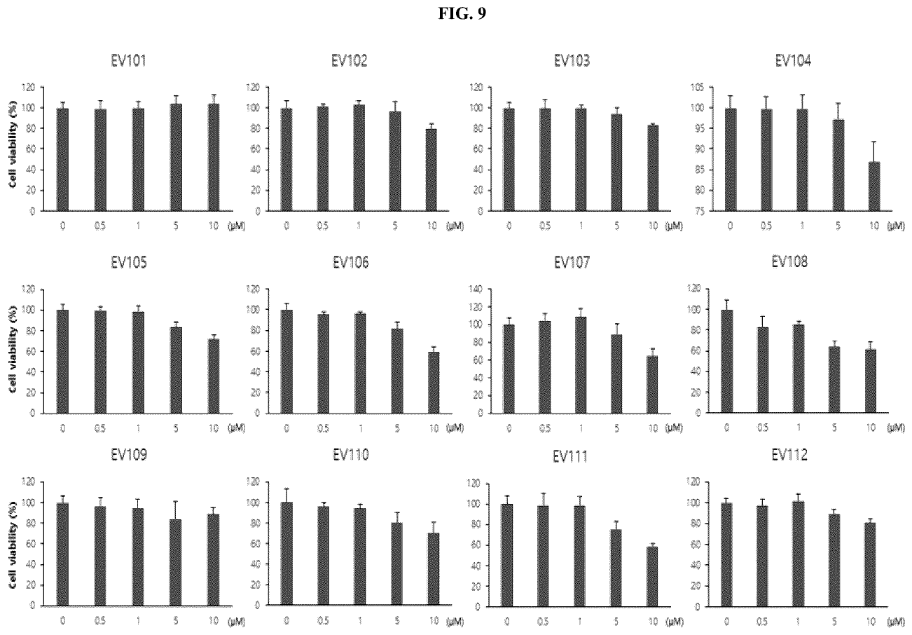

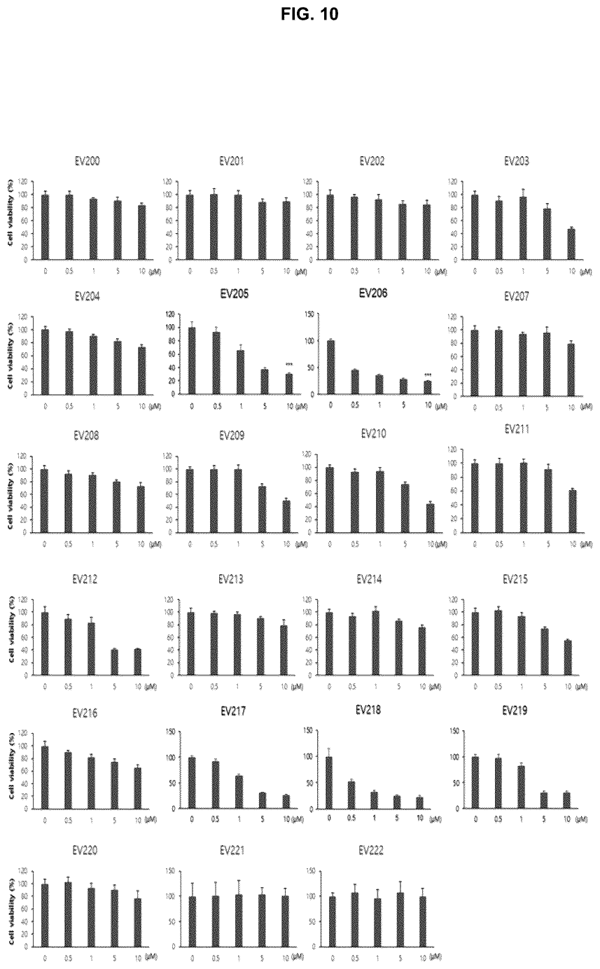

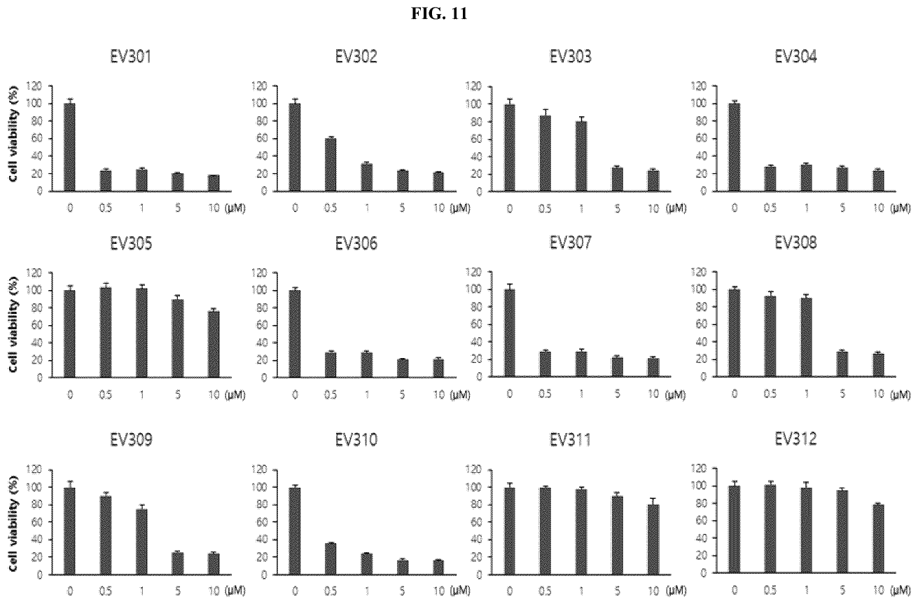

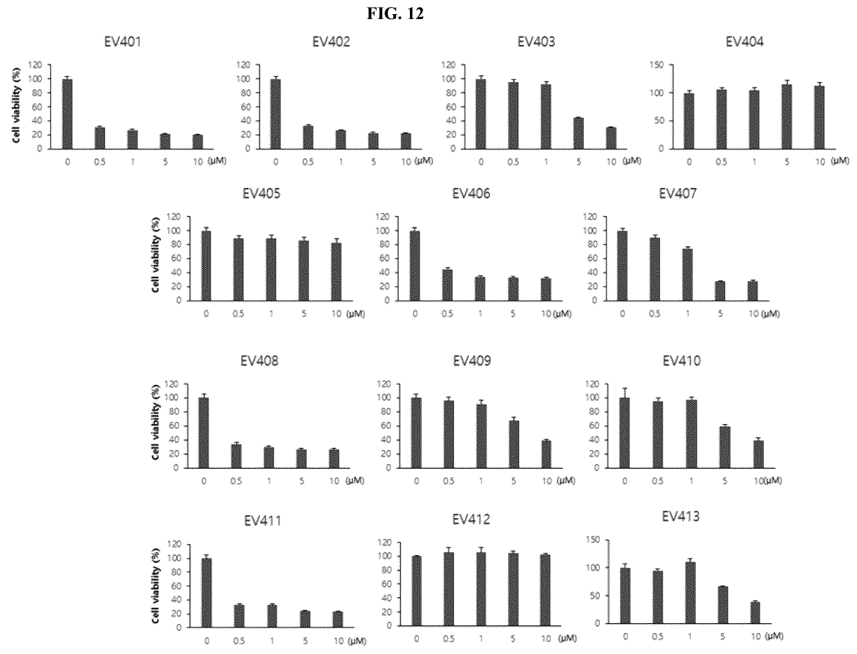

As shown in FIG. 9 to FIG. 12, it was confirmed that various evodiamine derivatives, in addition to EV507, showed an anti-cancer effect on lung cancer cells.

In particular, it was confirmed that EV205, EV206, EV217, EV218, EV219, EV301, EV302, EV303, EV304, EV306, EV307, EV308, EV309, EV310, EV401, EV402, EV403, EV406, EV407, EV408, EV411, and EV413 showed excellent effects in causing lung cancer cell death.

### Example 6. Confirmation of the Therapeutic Effect on Lung Cancer by EV206

Based on Example 5, the effects of EV206 on various lung cancer cells and chemotherapeutic agent-resistant lung cancer cell lines were confirmed.

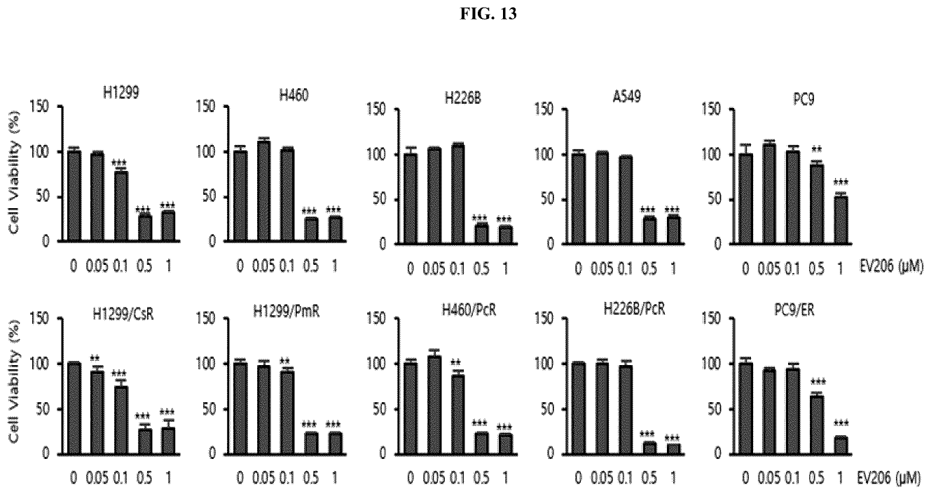

As shown in FIG. 13, it was confirmed that EV206 showed excellent growth-inhibitory effects on various lung cancer cell lines and chemotherapeutic agent-resistant lung cancer cell lines.

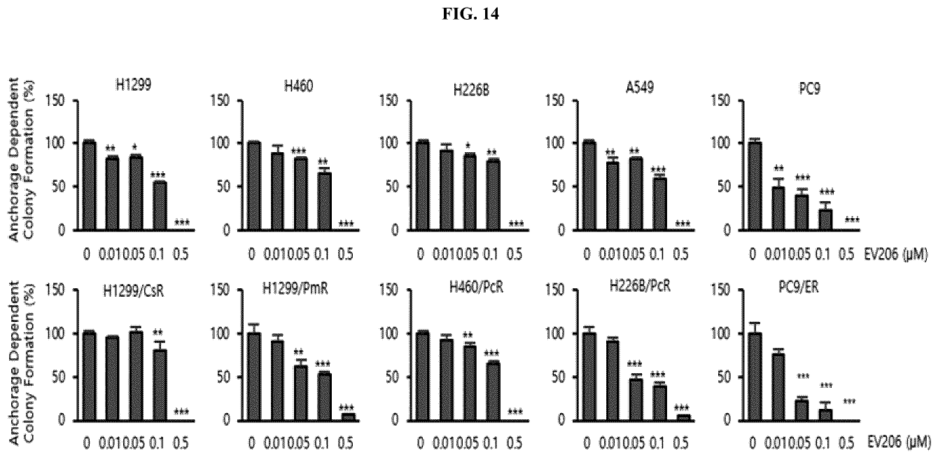

As shown in FIG. 14, EV206 significantly inhibited the formation of colonies under anchorage-dependent conditions at a low concentration of 0.5 μM.

### Example 7. Confirmation of the Therapeutic Effect on Lung Cancer by EV408

Based on Example 5, the effect of EV408 on various lung cancer cells and chemotherapeutic agent-resistant lung cancer cell lines was confirmed.

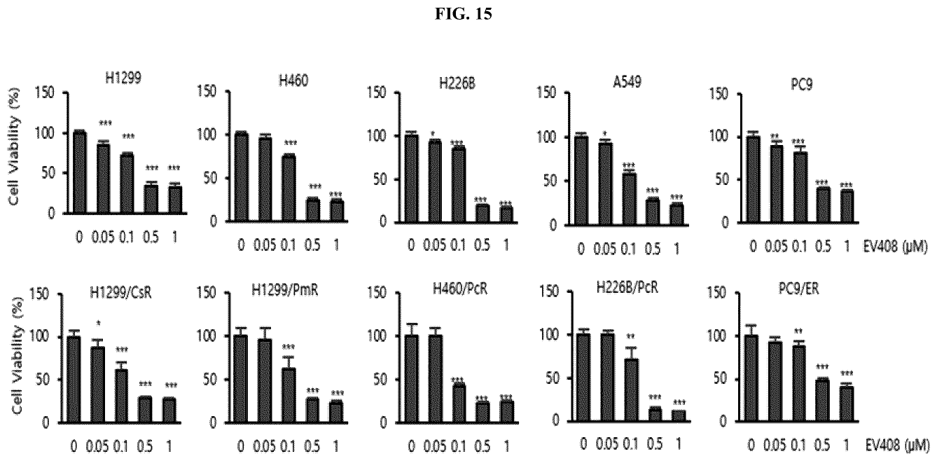

As shown in FIG. 15, it was confirmed that EV408 showed excellent growth-inhibitory effects on various lung cancer cell lines and chemotherapeutic agent-resistant lung cancer cell lines.

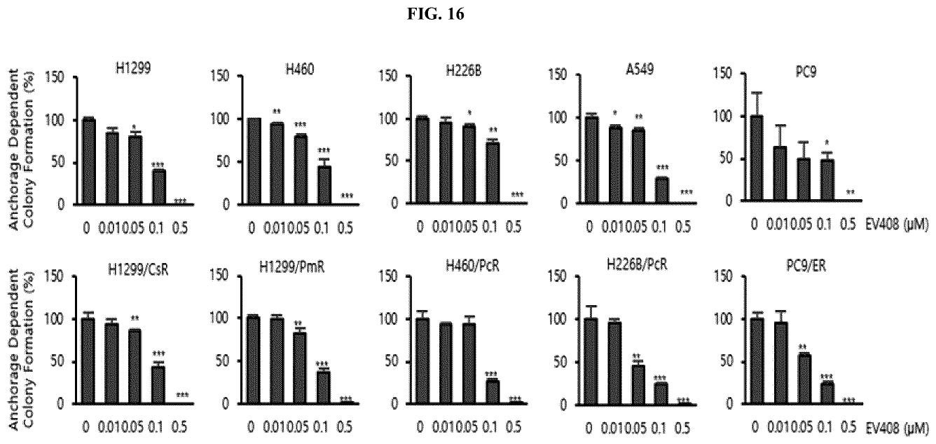

As shown in FIG. 16, EV408 significantly inhibited the formation of colonies under adherent conditions at a low concentration of 0.5 μM.

### Example 8. Confirmation of the Inhibition Effect of EV206 and EV408 on Hsp70 and Client Protein Expression

In the same way as in Example 3, the effect of EV507 on Hsp70 and related client proteins was confirmed.

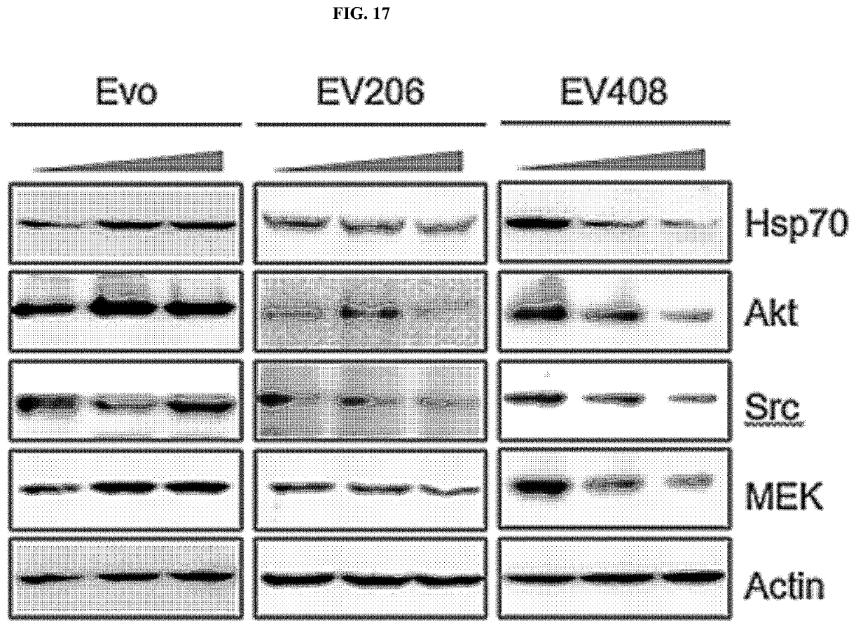

As shown in FIG. 17, when lung cancer cells were treated with EV206 and EV408, a clear reduction was observed in the expression of Hsp70 protein and Hsp70/Hsp90 client proteins (Akt, Src, and MEK).

### Example 9. Confirmation of the Therapeutic Effect on Lung Cancer by EV508 and EV509

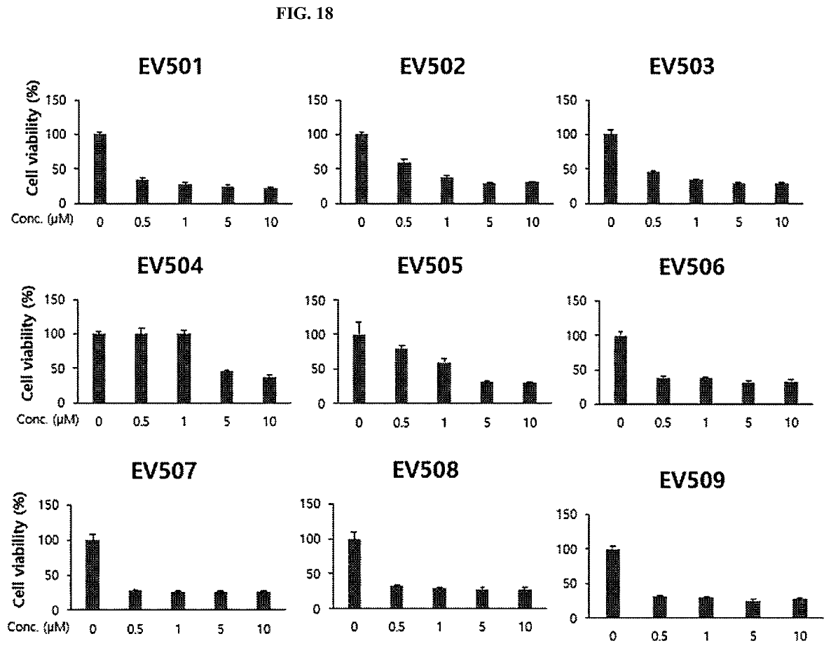

As shown in FIG. 18, among the developed evodiamine derivatives, EV501, EV507, EV508, and EV509 showed excellent anti-cancer efficacy by inhibiting over 65% of lung cancer cell survival at the lowest concentration of 0.5 μM, and EV502, EV503, and EV506 showed an effect of inhibiting over 50% of lung cancer cell survival at 0.5 μM.

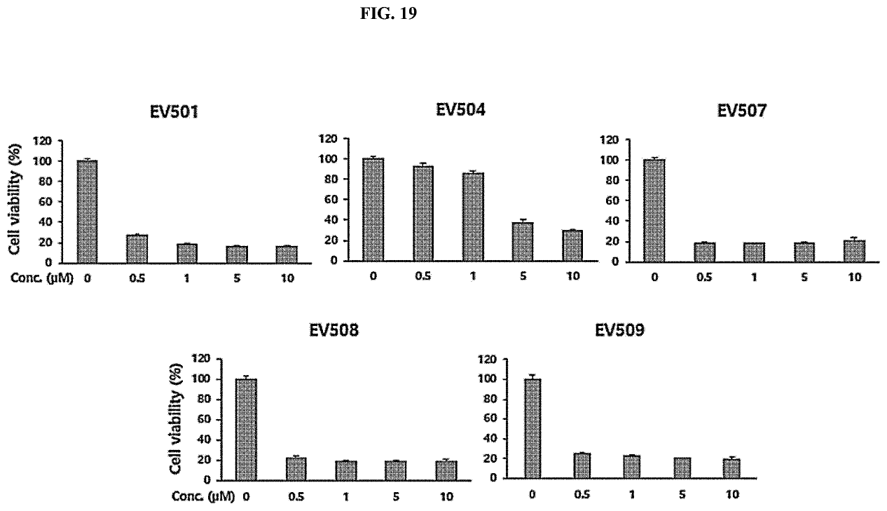

Also, as shown in FIG. 19, using the crystal violet assay, it was re-confirmed that the cancer cell survival inhibitory activity of the main evodiamine derivatives EV501, EV504, EV508, and EV509 was as excellent as EV507.

### Example 10. Inhibition Effect on Anchorage-Independent Colony Formation by EV501, EV507-EV509

The effect of evodiamine derivatives EV501, EV507-EV509 on the colony-forming ability of lung cancer cells under the anchorage-independent culture conditions was confirmed.

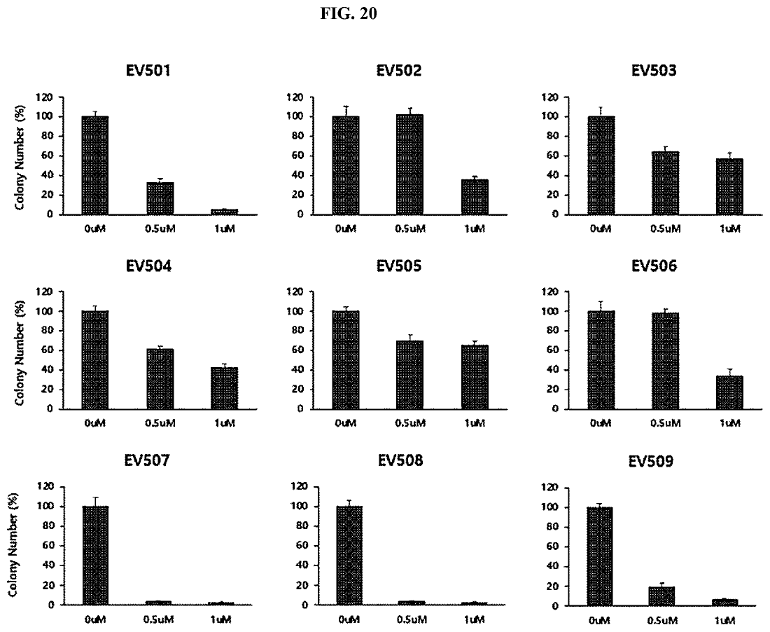

As shown in FIG. 20, it was confirmed that EV501, EV508, and EV509 effectively inhibited the anchorage-independent colony formation of lung cancer cells, just like EV507.

### Example 11. Confirmation of Hsp70 Inhibitory Activity of EV501

The effects of EV501 and EV507, one of the major evodiamine derivatives, on Hsp70 and Hsp70/Hsp90 client proteins were evaluated.

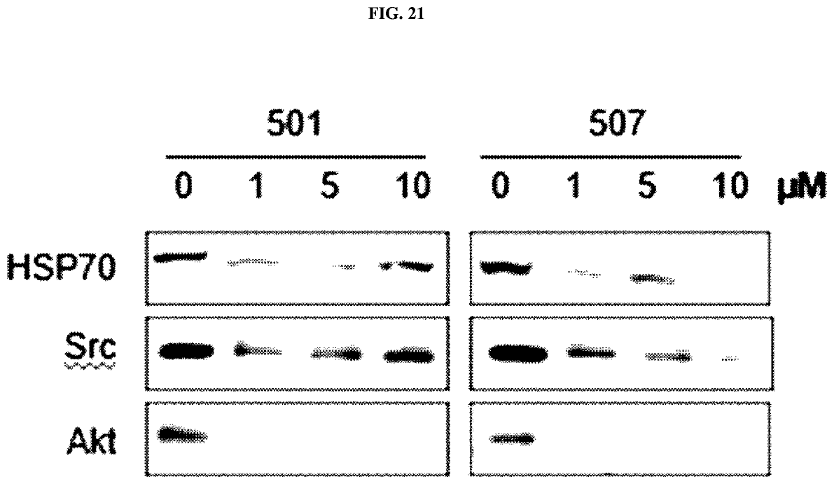

As shown in FIG. 21, evodiamine derivative EV501, similar to EV507, was confirmed to decrease the expression of Hsp70 and Src and Akt, which are client proteins of Hsp70/Hsp90.

The description of the present invention as stated above is for illustrative purposes only, and a person having ordinary skill in the art to which the present invention pertains will understand that the technical idea or essential characteristics of the present invention can be easily modified into other specific forms without changing them. Therefore, the examples described above should be understood as illustrative in all aspects and not limited.

### INDUSTRIAL APPLICABILITY

The indoloquinazolidine alkaloids of the present invention inhibit tumor growth, inhibit HSP70 protein expression and colony-forming ability of cancer cells, inhibit tumor growth in cancer cell line xenografts and patient-derived cancer xenograft mouse models, as well as inhibit the growth of drug-resistant cancer cells such as pemetrexed, cisplatin, and paclitaxel, and can be widely used for preventing and treating of various cancers, and are therefore industrially applicable.

1.-21. (canceled)

22. An indoloquinazolidine alkaloid represented by Chemical Formula 1 below, or a pharmaceutically acceptable salt thereof. 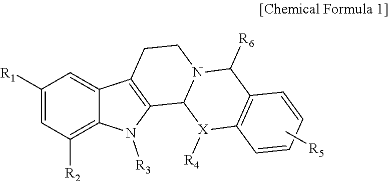
(In the Chemical Formula 1,
R₁ and R₂ are each independently hydrogen, substituted or unsubstituted C₁-C₆ alkyl, substituted or unsubstituted C₁-C₆ alkoxy, halogen, nitro (NO₂), amide, —NH-aryl, hydroxy, or —COOR₇,
R₃ is hydrogen, substituted or unsubstituted C₁-C₆ alkyl, —CH₂—R₈, substituted or unsubstituted C₁-C₆ acyl, substituted or unsubstituted aryl, —CO—R₈, or benzyl,
R₄ is hydrogen, substituted or unsubstituted C₁-C₆ alkyl, substituted or unsubstituted C₂-C₆ alkenyl, substituted or unsubstituted C₂-C₆ alkynyl, substituted or unsubstituted C₂-C₆ alkylalkenyl, substituted or unsubstituted C₂-C₆ alkylalkynyl, —CH₂—R₈, substituted or unsubstituted aryl, propargyl, substituted or unsubstituted C₁-C₆ acyl, or benzyl,
R₅ is hydrogen, substituted or unsubstituted C₁-C₆ alkyl, halogen, nitro, or substituted or unsubstituted C₁-C₆ alkoxy,
R₆ is hydrogen, oxygen, or substituted or unsubstituted C₁-C₆ alkyl,
R₇ and R₈ are each independently hydrogen, substituted or unsubstituted C₃-C₈ cycloalkyl, substituted or unsubstituted C₁-C₆ alkyl, or substituted or unsubstituted aryl,
X is C, N, O or S.)

23. The indoloquinazolidine alkaloid of claim 22, or a pharmaceutically acceptable salt thereof, wherein the R₁ and R₂ are each independently hydrogen, methoxy, butoxy, methyl, hydroxy, F, Cl, nitro, 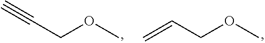
or —COOR₇,
R₃ is hydrogen, methyl, ethyl, propyl, butyl, hexyl, benzyl, —CO—R₈ or —CH₂—R₈,
R₄ is hydrogen, methyl, benzyl, substituted or unsubstituted C₂-C₅ alkenyl, substituted or unsubstituted C₂-C₅ alkynyl, substituted or unsubstituted C₂-C₅ alkylalkenyl, substituted or unsubstituted C₂-C₅ alkylalkynyl, cyclohexanecarbonyl, 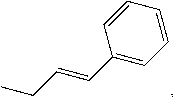
or —CH₂—R₈,
R₅ is hydrogen, methoxy, nitro, methyl, F, Cl, Br, or I,
R₆ is hydrogen, or oxygen,
R₇ and R₈ are each independently hydrogen, substituted or unsubstituted C₃-C₈ cycloalkyl, substituted or unsubstituted C₁-C₆ alkyl, or substituted or unsubstituted aryl, wherein the substituted aryl may be substituted with at least one selected from the group consisting of halogen, CN, C₁-C₆ haloalkyl, C₁-C₃ alkyl, C₁-C₃ alkoxy, and nitro group,
X is N, or O.

24. The indoloquinazolidine alkaloid of claim 22, or a pharmaceutically acceptable salt thereof, wherein the indoloquinazolidine alkaloid represented by the Chemical Formula 1 is selected from the group consisting of the following compounds.

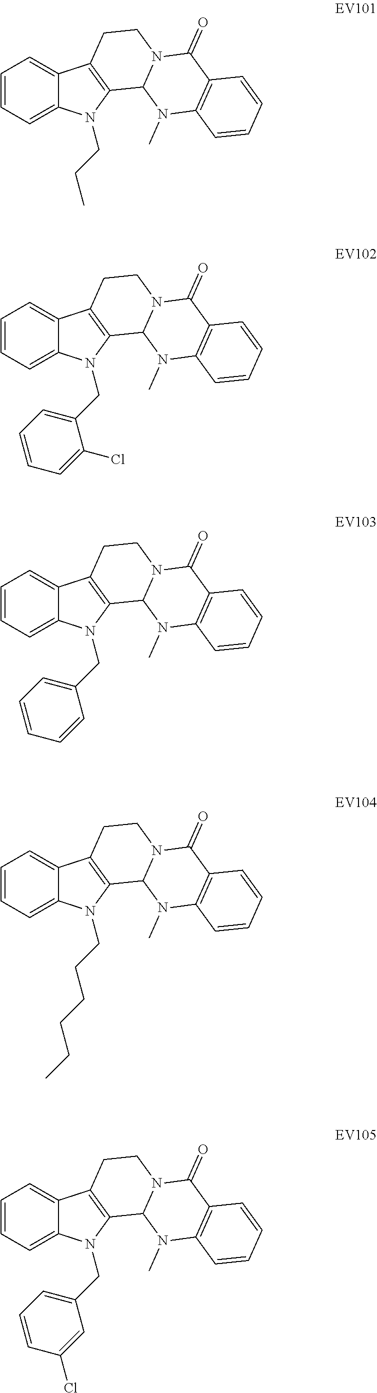 [SMILES: CCCn1c2c(c3ccccc31)CCN1C(=O)c3ccccc3N(C)C21 ; CN1c2ccccc2C(=O)N2CCc3c(n(Cc4ccccc4Cl)c4ccccc34)C21 ; CN1c2ccccc2C(=O)N2CCc3c(n(Cc4ccccc4)c4ccccc34)C21 ; CCCCCCn1c2c(c3ccccc31)CCN1C(=O)c3ccccc3N(C)C21 ; CN1c2ccccc2C(=O)N2CCc3c(n(Cc4cccc(Cl)c4)c4ccccc34)C21]

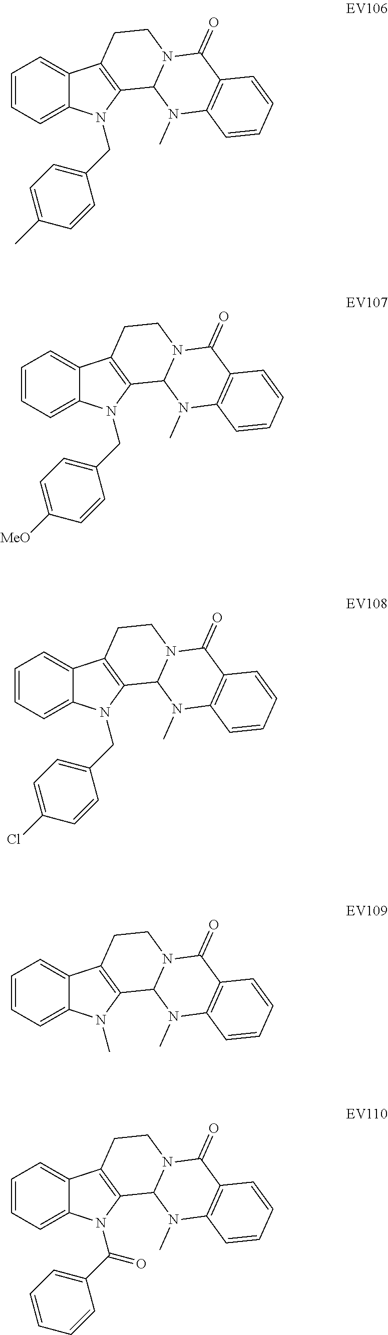 [SMILES: Cc1ccc(Cn2c3c(c4ccccc42)CCN2C(=O)c4ccccc4N(C)C32)cc1 ; COc1ccc(Cn2c3c(c4ccccc42)CCN2C(=O)c4ccccc4N(C)C32)cc1 ; CN1c2ccccc2C(=O)N2CCc3c(n(Cc4ccc(Cl)cc4)c4ccccc34)C21 ; CN1c2ccccc2C(=O)N2CCc3c(n(C)c4ccccc34)C21 ; CN1c2ccccc2C(=O)N2CCc3c(n(C(=O)c4ccccc4)c4ccccc34)C21]

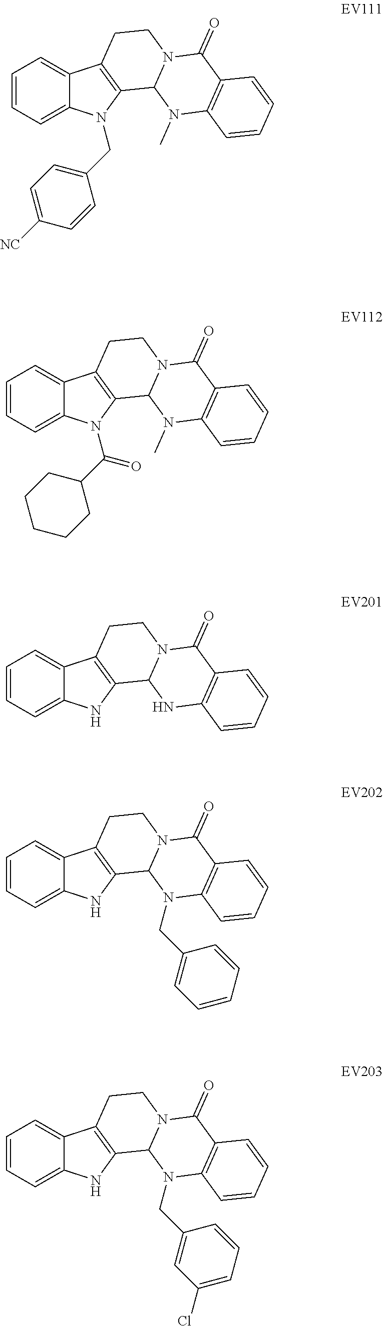 [SMILES: CN1c2ccccc2C(=O)N2CCc3c(n(Cc4ccc(C#N)cc4)c4ccccc34)C21 ; CN1c2ccccc2C(=O)N2CCc3c(n(C(=O)C4CCCCC4)c4ccccc34)C21 ; O=C1c2ccccc2NC2c3[nH]c4ccccc4c3CCN12 ; O=C1c2ccccc2N(Cc2ccccc2)C2c3[nH]c4ccccc4c3CCN12 ; O=C1c2ccccc2N(Cc2cccc(Cl)c2)C2c3[nH]c4ccccc4c3CCN12]

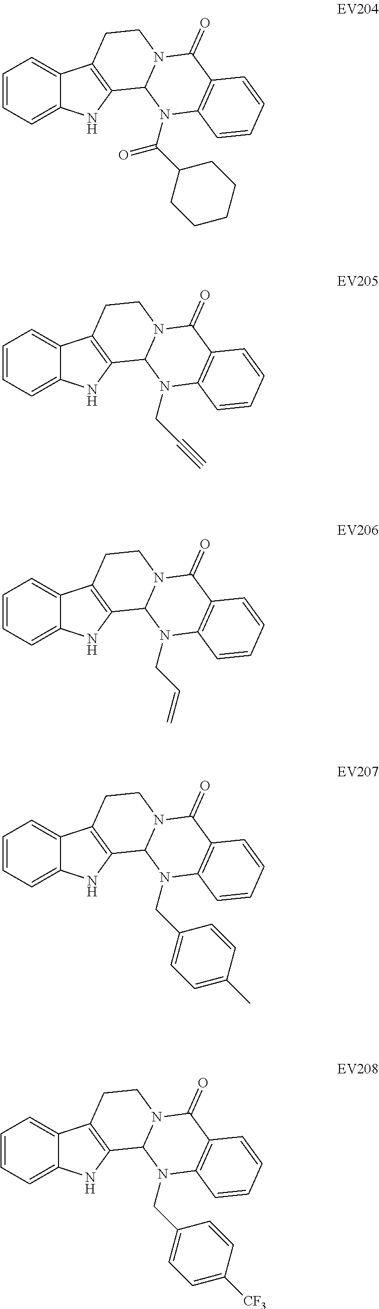 [SMILES: O=C1c2ccccc2N(C(=O)C2CCCCC2)C2c3[nH]c4ccccc4c3CCN12 ; C#CCN1c2ccccc2C(=O)N2CCc3c([nH]c4ccccc34)C21 ; C=CCN1c2ccccc2C(=O)N2CCc3c([nH]c4ccccc34)C21 ; Cc1ccc(CN2c3ccccc3C(=O)N3CCc4c([nH]c5ccccc45)C32)cc1 ; O=C1c2ccccc2N(Cc2ccc(C(F)(F)F)cc2)C2c3[nH]c4ccccc4c3CCN12]

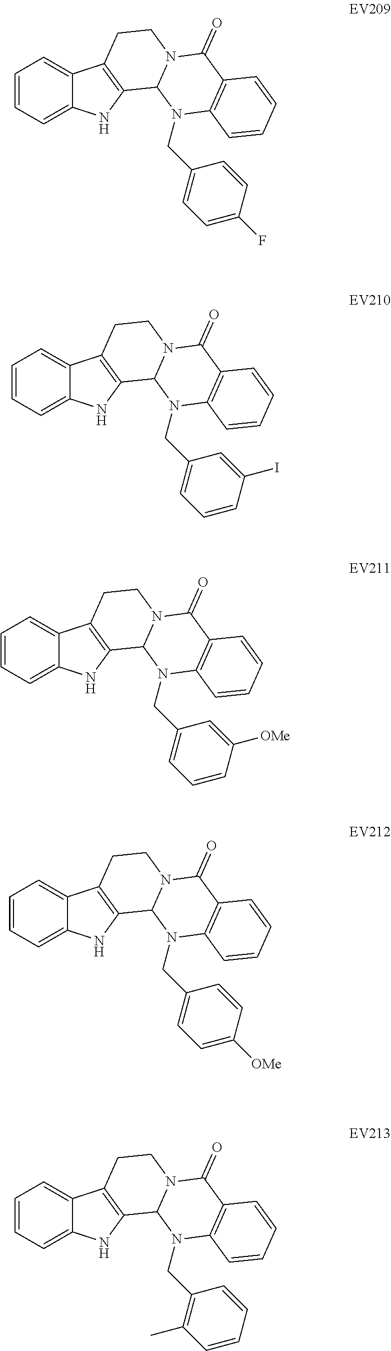 [SMILES: O=C1c2ccccc2N(Cc2ccc(F)cc2)C2c3[nH]c4ccccc4c3CCN12 ; O=C1c2ccccc2N(Cc2cccc(I)c2)C2c3[nH]c4ccccc4c3CCN12 ; COc1cccc(CN2c3ccccc3C(=O)N3CCc4c([nH]c5ccccc45)C32)c1 ; COc1ccc(CN2c3ccccc3C(=O)N3CCc4c([nH]c5ccccc45)C32)cc1 ; Cc1ccccc1CN1c2ccccc2C(=O)N2CCc3c([nH]c4ccccc34)C21]

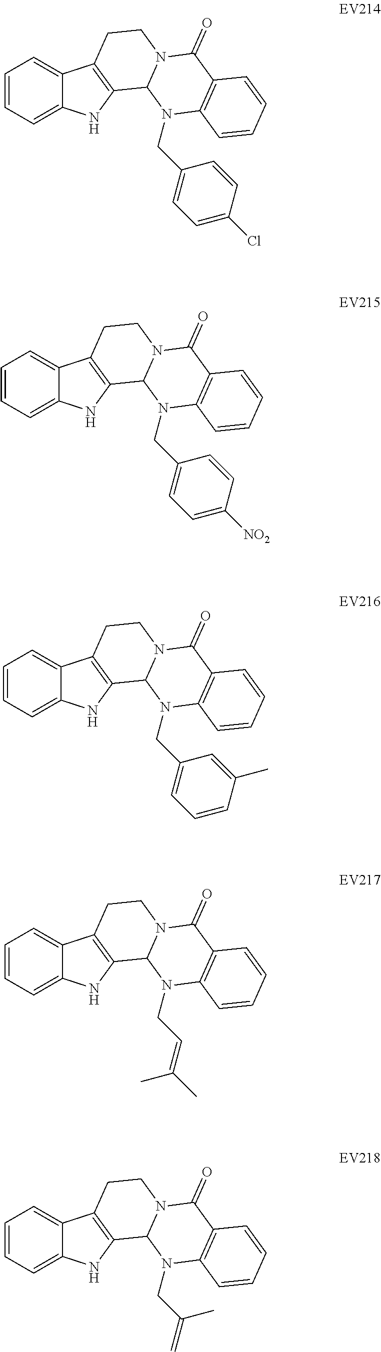 [SMILES: O=C1c2ccccc2N(Cc2ccc(Cl)cc2)C2c3[nH]c4ccccc4c3CCN12 ; O=C1c2ccccc2N(Cc2ccc([N+](=O)[O-])cc2)C2c3[nH]c4ccccc4c3CCN12 ; Cc1cccc(CN2c3ccccc3C(=O)N3CCc4c([nH]c5ccccc45)C32)c1 ; CC(C)=CCN1c2ccccc2C(=O)N2CCc3c([nH]c4ccccc34)C21 ; C=C(C)CN1c2ccccc2C(=O)N2CCc3c([nH]c4ccccc34)C21]

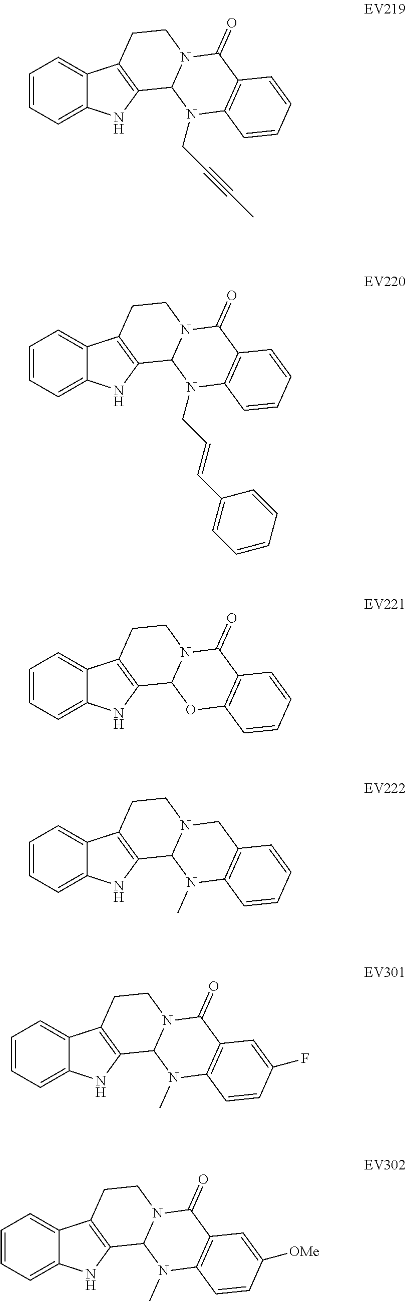 [SMILES: CC#CCN1c2ccccc2C(=O)N2CCc3c([nH]c4ccccc34)C21 ; O=C1c2ccccc2N(C/C=C/c2ccccc2)C2c3[nH]c4ccccc4c3CCN12 ; O=C1c2ccccc2OC2c3[nH]c4ccccc4c3CCN12 ; CN1c2ccccc2CN2CCc3c([nH]c4ccccc34)C21 ; CN1c2ccc(F)cc2C(=O)N2CCc3c([nH]c4ccccc34)C21 ; COc1ccc2c(c1)C(=O)N1CCc3c([nH]c4ccccc34)C1N2C]

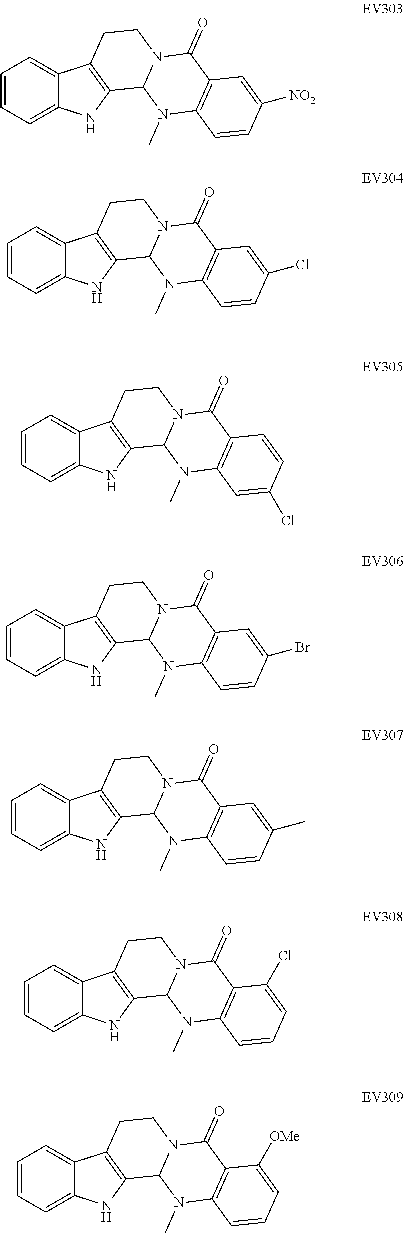

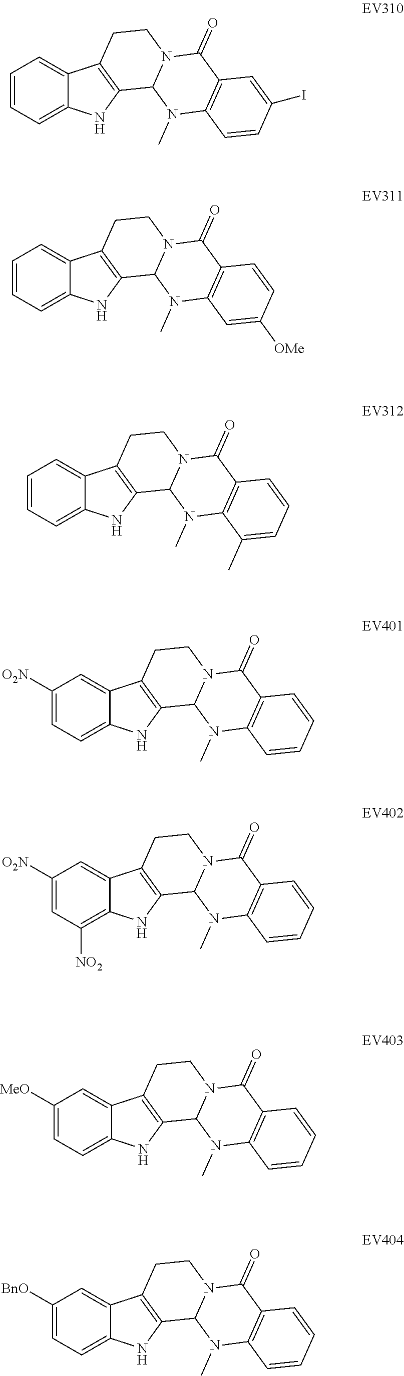

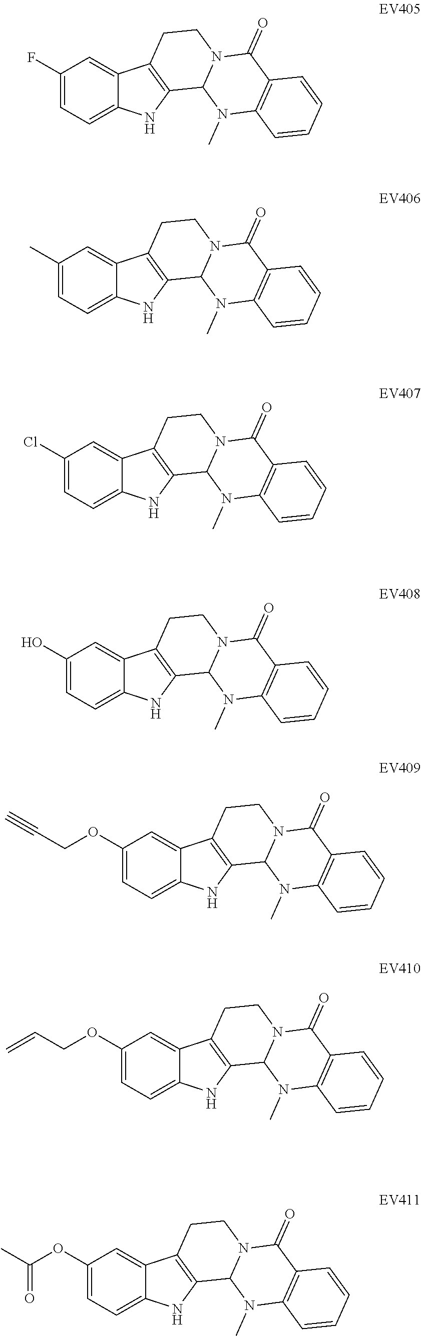

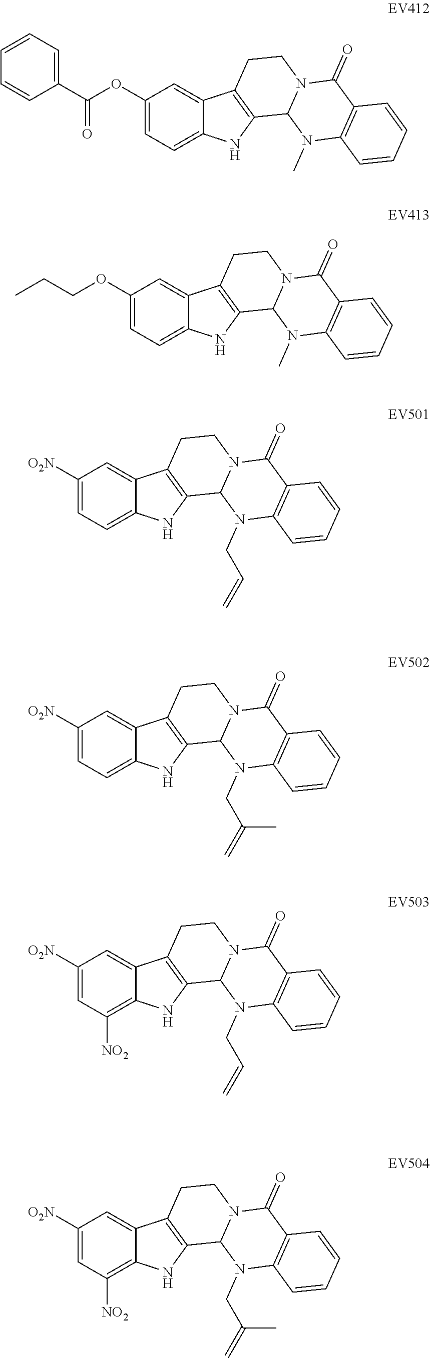 [SMILES: CN1c2ccccc2C(=O)N2CCc3c([nH]c4ccc(OC(=O)c5ccccc5)cc34)C21 ; CCCOc1ccc2[nH]c3c(c2c1)CCN1C(=O)c2ccccc2N(C)C31 ; C=CCN1c2ccccc2C(=O)N2CCc3c([nH]c4ccc([N+](=O)[O-])cc34)C21 ; C=C(C)CN1c2ccccc2C(=O)N2CCc3c([nH]c4ccc([N+](=O)[O-])cc34)C21 ; C=CCN1c2ccccc2C(=O)N2CCc3c([nH]c4c([N+](=O)[O-])cc([N+](=O)[O-])cc34)C21 ; C=C(C)CN1c2ccccc2C(=O)N2CCc3c([nH]c4c([N+](=O)[O-])cc([N+](=O)[O-])cc34)C21]

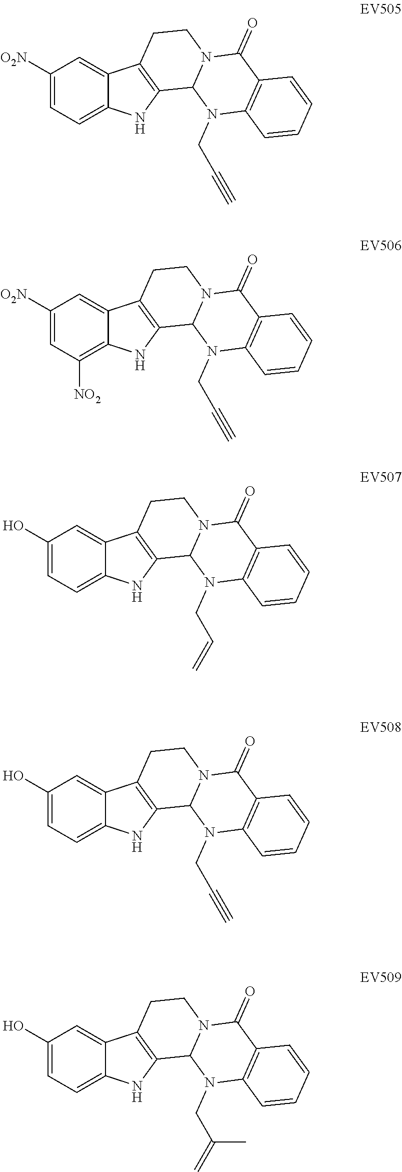 [SMILES: C#CCN1c2ccccc2C(=O)N2CCc3c([nH]c4ccc([N+](=O)[O-])cc34)C21 ; C#CCN1c2ccccc2C(=O)N2CCc3c([nH]c4c([N+](=O)[O-])cc([N+](=O)[O-])cc34)C21 ; C=CCN1c2ccccc2C(=O)N2CCc3c([nH]c4ccc(O)cc34)C21 ; C#CCN1c2ccccc2C(=O)N2CCc3c([nH]c4ccc(O)cc34)C21 ; C=C(C)CN1c2ccccc2C(=O)N2CCc3c([nH]c4ccc(O)cc34)C21]

25. A pharmaceutical composition comprising an indoloquinazolidine alkaloid of claim 22 or a pharmaceutically acceptable salt thereof as an active ingredient.

26. The pharmaceutical composition of claim 25, wherein the composition comprises at least one more selected from the group consisting of pemetrexed, cisplatin, and paclitaxel.

27. An anti-cancer adjuvant that enhances the anti-cancer efficacy of an anti-cancer drug comprising an indoloquinazolidine alkaloid of claim 22, or a pharmaceutically acceptable salt thereof as an active ingredient.

28. The anti-cancer adjuvant of claim 27, wherein the anti-cancer drug is at least one selected from the group consisting of pemetrexed, cisplatin, and paclitaxel.

29. A method for treating cancer, comprising the step of administering a pharmaceutical composition to a subject in need thereof, wherein the pharmaceutical composition comprises an indoloquinazolidine alkaloid of claim 22 or a pharmaceutically acceptable salt thereof as an active ingredient.

30. The method of claim 29, wherein the cancer is at least one selected from the group consisting of cervical cancer, lung cancer, pancreatic cancer, non-small cell lung cancer, liver cancer, colon cancer, colorectal cancer, bone cancer, skin cancer, head and neck cancer, cutaneous melanoma, intraocular melanoma, uterine cancer, ovarian cancer, rectal cancer, liver cancer, brain tumor, blood cancer, gastric cancer, anal cancer, breast cancer, fallopian tube cancer, endometrial cancer, vaginal cancer, vulvar cancer, Hodgkin's disease, esophageal cancer, small intestine cancer, endocrine gland cancer, thyroid cancer, parathyroid cancer, adrenal cancer, soft tissue sarcoma, urethral cancer, penile cancer, prostate cancer, bladder cancer, kidney cancer, ureteral cancer, renal cell carcinoma, renal pelvis carcinoma, central nervous system (CNS) tumor, primary CNS lymphoma, spinal cord tumor, brain stem glioma and pituitary adenoma.

31. The method of claim 29, wherein the cancer is lung cancer.

32. The method of claim 29, wherein the indoloquinazolidine alkaloid reduces at least one selected from the group consisting of the number and volume of tumors.

33. The method of claim 29, wherein the indoloquinazolidine alkaloid suppresses the expression of HSP70 protein.

34. The method of claim 29, wherein the indoloquinazolidine alkaloid suppresses the expression of at least one protein selected from the group consisting of Akt, Src, and MEK.

35. The method of claim 29, wherein the composition is for preventing or treating lung cancer that has acquired resistance to an anti-cancer drug.

36. A method for enhancing or promoting the anti-cancer efficacy of an anti-cancer drug, wherein the method comprises administering an anti-cancer adjuvant comprising the indoloquinazolidine alkaloid of claim 22 or a pharmaceutically acceptable salt thereof as an active ingredient to a subject in need thereof.
